(19) United States
(12) Patent Application Publication
(10) Pub. No.: US 2005/0239778 A1
(43) Pub. Date: Oct. 27, 2005

(54) Novel medicament combinations for the treatment of respiratory diseases

(75) Inventors: Ingo Konetzki, Warthausen (DE); Thierry Bouyssou, Mietingen (DE); Philipp Lustenberger, Warthausen (DE); Michael P. Pieper, Biberach (DE); Andreas Schnapp, Biberach (DE); Christoph Hoenke, Ingelheim (DE); Sabine Pestel, Attenweiler (DE); Klaus Rudolf, Warthausen (DE); Michel Pairet, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH

(21) Appl. No.: 11/109,094

(22) Filed: Apr. 19, 2005

Related U.S. Application Data
(60) Provisional application No. 60/578,542, filed on Jun. 10, 2004.

(30) Foreign Application Priority Data
Apr. 22, 2004 (DE) 10 2004 019 540.4
Nov. 3, 2004 (DE) 10 2004 052 987.6
Feb. 7, 2005 (EP) EP 05002496.7

(51) Int. Cl. A61K 31/538; A61K 31/56

(52) U.S. Cl. 514/230.5; 514/171

(57) ABSTRACT

The present invention relates to new medicament combinations which contain in addition to one or more, preferably one, compound of general formula 1 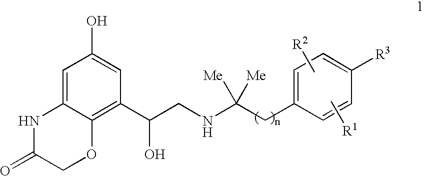
wherein the groups R¹, R² and R³ may have the meanings given in the claims and in the specification, at least one other active substance 2, processes for preparing them and their use as pharmaceutical compositions.

This application claims priority benefit under 35 USC 119(e) from U.S. Provisional Application 60/578,542, filed Jun. 10, 2004, from DE 10 2004 019 540.4, filed Apr. 22, 2004, from DE 10 2004 052 987.6, filed Nov. 3, 2004, and from EP 05002496.7, filed Feb. 7, 2005.

The present invention relates to new medicament combinations which contain in addition to one or more, preferably one, compound of general formula 1 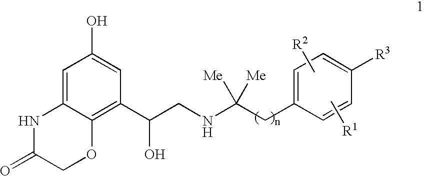
wherein the groups R¹, R² and R³ may have the meanings given in the claims and specification, at least one other active substance 2, processes for preparing them and their use as pharmaceutical compositions.

### DETAILED DESCRIPTION OF THE INVENTION

The present invention relates to medicament combinations which contain in addition to one or more, preferably one, compound of general formula 1 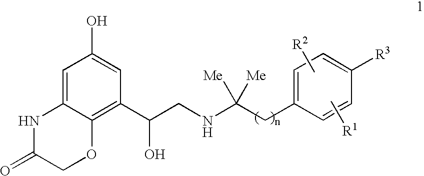
wherein
n denotes 1 or 2, preferably 1;
R¹ denotes hydrogen, halogen, C₁-C₄-alkyl or —O—C₁-C₄-alkyl;
R² denotes hydrogen, halogen, C₁-C₄-alkyl or —O—C₁—C₄-alkyl;
R³ denotes C₁-C₄-alkyl, OH, halogen, —O—C₁-C₄-alkyl, —O—C₁-C₄-alkylene-COOH, —O—C₁-C₄-alkylene-CO—O—C₁-C₄-alkyl, at least one other active substance 2.

Preferably the present invention relates to medicament combinations, which contain, in addition to one or more, preferably one, compound of formula 1 as a further active substance 2 one or more compounds selected from the categories of the anticholinergics (2a), PDE IV inhibitors (2b), steroids (2c), LTD4-antagonists (2d) and EGFR-inhibitors (2e).

Preferred are medicament combinations which contain in addition to one or more, preferably one, compound of general formula 1, wherein
n denotes 1 or 2, preferably 1;
R¹ denotes hydrogen, fluorine, chlorine, methyl or methoxy;
R² denotes hydrogen, fluorine, chlorine, methyl or methoxy;
R³ denotes C₁-C₄-alkyl, OH, fluorine, chlorine, bromine, —O—C₁-C₄-alkyl, —O—C₁-C₄-alkylene-COOH, —O—C₁-C₄-alkylene-CO—O—C₁-C₄-alkyl,
   at least one other active substance 2.

Also preferred are medicament combinations which contain in addition to one or more, preferably one, compound of general formula 1, wherein
n denotes 1;
R¹ denotes hydrogen or C₁-C₄-alkyl;
R² denotes hydrogen or C₁-C₄-alkyl;
R³ denotes C₁-C₄-alkyl, OH, —O—C₁-C₄-alkyl, —O—C₁-C₄-alkylene-COOH or —O—C₁-C₄-alkylene-CO—O—C₁-C₄-alkyl,
   at least one other active substance 2.

Also preferred are medicament combinations which contain in addition to one or more, preferably one, compound of general formula 1, wherein
n denotes 1;
R¹ denotes hydrogen, methyl or ethyl;
R² denotes hydrogen, methyl or ethyl;
R³ denotes methyl, ethyl, OH, methoxy, ethoxy, —O—CH₂—COOH, —O—CH₂—COOmethyl or —O—CH₂—COOethyl,
   at least one other active substance 2.

Also preferred are medicament combinations which contain in addition to one or more, preferably one, compound of general formula 1, wherein
n denotes 1;
R¹ denotes hydrogen or methyl;
R² denotes hydrogen or methyl;
R³ denotes methyl, OH, methoxy, —O—CH₂—COOH or —O—CH₂—COOethyl,
   at least one other active substance 2.

Preferred medicament combinations according to the invention are those which contain in addition to one or more, preferably one, compound of general formula 1, wherein
R³ denotes methoxy, ethoxy, —O—CH₂—COOH, —O—CH₂—COOmethyl or —O—CH₂—COOethyl,
   and R¹, R² and n may have the meanings given above, at least one other active substance 2.

The present invention also relates to medicament combinations which contain in addition to one or more, preferably one, compound of general formula 1, wherein
n denotes 1;
R¹ denotes halogen, C₁-C₄-alkyl or —O—C₁-C₄-alkyl;
R² denotes halogen, C₁-C₄-alkyl or —O—C₁-C₄-alkyl;
R³ denotes halogen, C₁-C₄-alkyl or —O—C₁-C₄-alkyl, at least one other active substance 2.

The present invention also relates to medicament combinations which contain in addition to one or more, preferably one, compound of general formula 1, wherein
n denotes 1;
R¹ denotes fluorine, chlorine, methyl or methoxy;
R² denotes fluorine, chlorine, methyl or methoxy;
R³ denotes fluorine, chlorine, methyl or methoxy
   at least one other active substance 2.

In another preferred aspect the present invention relates to medicament combinations which contain in addition to one or more, preferably one, compound of general formula 1, wherein
n denotes 1;
R¹ denotes hydrogen;
R² denotes hydrogen, fluorine, chlorine or methyl;
R³ denotes methyl, ethyl, iso-propyl, tert.-butyl, OH, fluorine, chlorine, bromine, methoxy, ethoxy, —O—CH₂—COOH, —O—CH₂—CH₂—COOH, —O—CH₂—CH₂—CH₂—COOH, O—CH₂—COOmethyl, —O—CH₂—COOethyl, —O—CH₂—CH₂—COOmethyl, —O—CH₂—CH₂—COOethyl, —O—CH₂—CH₂—CH₂—COOmethyl, —O—CH₂—CH₂—CH₂—COOethyl,
   at least one other active substance 2.

Particularly preferred are medicament combinations which contain in addition to one or more, preferably one, compound of general formula 1 wherein
n denotes 1;
R¹ denotes hydrogen;
R² denotes hydrogen, fluorine, chlorine or methyl;
R³ denotes OH, fluorine, chlorine, methyl, methoxy, ethoxy or —O—CH₂—COOH, at least one other active substance 2.

Also preferred are those medicament combinations which contain in addition to one or more, preferably one compound of general formula 1, wherein
n denotes 1;
R¹ denotes hydrogen;
R² denotes halogen, C₁-C₄-alkyl or —O—C₁—C₄-alkyl, preferably fluorine, chlorine, methoxy or methyl;
R³ denotes halogen, C₁-C₄-alkyl or —O—C₁-C₄-alkyl, preferably fluorine, chlorine, methoxy or methyl,
   at least one other active substance 2.

In another preferred aspect the present invention relates to medicament combinations which contain in addition to one or more, preferably one, compound of general formula 1, wherein n=1, R¹ and R² denote hydrogen and the group R³ may have the meanings given above, at least one other active substance 2.

In another preferred aspect the present invention relates to medicament combinations which contain in addition to one or more, preferably one, compound of general formula 1, wherein
n denotes 1;
R¹ and R² denote hydrogen;
R³ denotes methyl, ethyl, iso-propyl, tert.-butyl, OH, fluorine, chlorine, bromine, methoxy, ethoxy, —O—CH₂—COOH, —O—CH₂—CH₂—COOH, —O—CH₂—CH₂—CH₂—COOH, —O—CH₂—COOmethyl, —O—CH₂—COOethyl, —O—CH₂—CH₂—COOmethyl, —O—CH₂—CH₂—COOethyl, —O—CH₂—CH₂—CH₂—COOmethyl, —O—CH₂—CH₂—CH₂—COOethyl,
   at least one other active substance 2.

Particularly preferred are medicament combinations which contain in addition to one or more, preferably one, compound of general formula 1, wherein
n denotes 1;
R¹ and R² denotes hydrogen;
R³ denotes OH, fluorine, chlorine, methoxy, ethoxy, —O—CH₂—COOH, preferably OH, fluorine, chlorine, ethoxy or methoxy,
   at least one other active substance 2.

Particularly preferred are medicament combinations which contain in addition to one or more, preferably one, compound of general formula 1, wherein
n denotes 1;
R¹ and R² denotes hydrogen;
R³ denotes fluorine, chlorine, methoxy or ethoxy,
   at least one other active substance 2.

The present invention also relates to medicament combinations which contain in addition to one or more, preferably one, compound of general formula 1, wherein
n denotes 1;
R¹ denotes hydrogen, halogen, C₁-C₄-alkyl or —O—C₁-C₄-alkyl;
R² denotes hydrogen, halogen, C₁-C₄-alkyl or —O—C₁—C₄-alkyl;
R³ denotes hydrogen,
   at least one other active substance 2.

Preferred are medicament combinations which contain in addition to one or more, preferably one, compound of general formula 1, wherein
n denotes 1;
R¹ denotes hydrogen, fluorine, chlorine, methyl or methoxy;
R² denotes hydrogen, fluorine, chlorine, methyl or methoxy;
R³ denotes hydrogen,
   at least one other active substance 2.

The present invention also relates to medicament combinations which contain in addition to one or more, preferably one, compound of general formula 1, wherein
n denotes 1;
R¹ denotes fluorine, chlorine, methyl or methoxy;
R² denotes fluorine, chlorine, methyl or methoxy;
R³ denotes hydrogen,
   at least one other active substance 2.

In the compounds of formula 1 the groups R¹ and R², if they do not represent hydrogen, may each be in the ortho or meta position relative to the bond to the benzylic “—CH₂”— group. If neither of the groups R¹ and R² denotes hydrogen, preferred compounds of formula 1 for the medicament combinations according to the invention are those wherein either the two groups R¹ and R² are in the ortho configuration or both groups R¹ and R² are in the meta configuration, while compounds in which both groups R¹ and R² are in the ortho configuration are of particular importance.

In the compounds of formula 1 wherein one of the groups R¹ and R² does not denote hydrogen, it may be in the ortho or meta position relative to the bond to the benzylic “—CH₂” group. In this case particularly preferred compounds of formula 1 for the medicament combinations according to the invention are those wherein the group R¹ or R² which does not denote hydrogen is in the ortho configuration.

Also particularly preferred are medicament combinations which contain in addition to one or more, preferably one, compound of general formula 1 selected from the compounds
6-hydroxy-8-{1-hydroxy-2-[2-(4-methoxy-phenyl)-1,1-dimethyl-ethylamino]-ethyl}-4H-benzo[1,4]oxazin-3-one (1.1);
6-hydroxy-8-{1-hydroxy-2-[2-(4-phenoxyethyl-acetate)-1,1-dimethyl-ethylamino]-ethyl}-4H-benzo[1,4]oxazin-3-one (1.2);
6-hydroxy-8-{1-hydroxy-2-[2-(4-phenoxy-acetic acid)-1,1-dimethyl-ethylamino]-ethyl}-4H-benzo[1,4]oxazin-3-one (1.3);
8-{2-[1,1-dimethyl-2-(2,4,6-trimethylphenyl)-ethylamino]-1-hydroxy-ethyl}-6-hydroxy-4H-benzo[1,4]oxazin-3-one (1.4);
6-hydroxy-8-{1-hydroxy-2-[2-(4-hydroxy-phenyl)-1,1-dimethyl-ethylamino]-ethyl}-4H-benzo[1,4]oxazin-3-one (1.5);
6-hydroxy-8-{1-hydroxy-2-[2-(4-isopropyl-phenyl)-1,1dimethyl-ethylamino]-ethyl}-4H-benzo[1,4]oxazin-3-one (1.6);
8-{2-[2-(4-ethyl-phenyl)-1,1-dimethyl-ethylamino]-1-hydroxy-ethyl}-6-hydroxy-4H-benzo[1,4]oxazin-3-one (1.7);
   8-{2-[2-(4-fluoro-3-methyl-phenyl)-1,1-dimethyl-ethylamino]-1-hydroxy-ethyl}-6-hydroxy-4H-benzo[1,4]oxazin-3-one (1.8);
8-{2-[2-(4-fluoro-2-methyl-phenyl)-1,1-dimethyl-ethylamino]-1-hydroxy-ethyl}-6-hydroxy-4H-benzo[1,4]oxazin-3-one (1.9);
8-{2-[2-(2,4-difluoro-phenyl)-1,1-dimethyl-ethylamino]-1-hydroxy-ethyl}-6-hydroxy-4H-benzo[1,4]oxazin-3-one (1.10);
8-{2-[2-(3,5-difluoro-phenyl)-1,1-dimethyl-ethylamino]-1-hydroxy-ethyl}-6-hydroxy-4H-benzo[1,4]oxazin-3-one (1.11);
8-{2-[2-(4-ethoxy-phenyl)-1,1-dimethyl-ethylamino]-1-hydroxy-ethyl}-6-hydroxy-4H-benzo[1,4]oxazin-3-one (1.12);
8-{2-[2-(3,5-dimethyl-phenyl)-1,1-dimethyl-ethylamino]-1-hydroxy-ethyl}-6-hydroxy-4H-benzo[1,4]oxazin-3-one (1.13);
4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid (1.14);
8-{2-[2-(3,4-difluoro-phenyl)-1,1-dimethyl-ethylamino]-1-hydroxy-ethyl}-6-hydroxy-4H-benzo[1,4]oxazin-3-one (1.15);
8-{2-[2-(2-chloro-4-fluoro-phenyl)-1,1-dimethyl-ethylamino]-1-hydroxy-ethyl}-6-hydroxy-4H-benzo[1,4]oxazin-3-one (1.16);
8-{2-[2-(4-chloro-phenyl)-1,1-dimethyl-ethylamino]-1-hydroxy-ethyl}-6-hydroxy-4H-benzo[1,4]oxazin-3-one (1.17);
8-{2-[2-(4-bromo-phenyl)-1,1-dimethyl-ethylamino]-1-hydroxy-ethyl}-6-hydroxy-4H-benzo[1,4]oxazin-3-one (18);
8-{2-[2-(4-fluoro-phenyl)-1,1-dimethyl-ethylamino]-1-hydroxy-ethyl}-6-hydroxy-4H-benzo[1,4]oxazin-3-one (1.19);
8-{2-[2-(4-fluoro-3-methoxy-phenyl)-1,1-dimethyl-ethylamino]-1-hydroxy-ethyl}-6-hydroxy-4H-benzo[1,4]oxazin-3-one 20 );
8-{2-[2-(4-fluoro-2,6-dimethyl-phenyl)-1,1-dimethyl-ethylamino]-1-hydroxy-ethyl}-6-hydroxy-4H-benzo[1,4]oxazin-3-one (1.21);
8-{2-[2-(4-chloro-2-methyl-phenyl)-1,1-dimethyl-ethylamino]-1-hydroxy-ethyl}-6-hydroxy-4H-benzo[1,4]oxazin-3-one (1.22);
8-{2-[2-(4-chloro-3-fluoro-phenyl)-1,1-dimethyl-ethylamino]-1-hydroxy-ethyl}-6-hydroxy-4H-benzo[1,4]oxazin-3-one (23);
8-{2-[2-(4-chloro-2-fluoro-phenyl)-1,1-dimethyl-ethylamino]-1-hydroxy-ethyl}-6-hydroxy-4H-benzo[1,4]oxazin-3-one (1.24);
8-{2-[2-(3-chloro-4-fluoro-phenyl)-1,1-dimethyl-ethylamino]-1-hydroxy-ethyl}-6-hydroxy-4H-benzo[1,4]oxazin-3-one (1.25);
8-{2-[2-(2,6-difluoro-4-methoxy-phenyl)-1,1-dimethyl-ethylamino]-1-hydroxy-ethyl}-6-hydroxy-4H-benzo[1,4]oxazin-3-one (1.26);
8-{2-[2-(2,5-difluoro-4-methoxy-phenyl)-1,1-dimethyl-ethylamino]-1-hydroxy-ethyl}-6-hydroxy-4H-benzo[1,4]oxazin-3-one (1.27);
8-{2-[2-(4-fluoro-3,5-dimethyl-phenyl)-1,1-dimethyl-ethylamino]-1-hydroxy-ethyl}-6-hydroxy-4H-benzo[1,4]oxazin-3-one (1.28);
8-{2-[2-(3,5-dichloro-phenyl)-1,1-dimethyl-ethylamino]-1-hydroxy-ethyl}-6-hydroxy-4H-benzo[1,4]oxazin-3-one (1.29);
8-{2-[2-(4-chloro-3-methyl-phenyl)-1,1-dimethyl-ethylamino]-1-hydroxy-ethyl}-6-hydroxy-4H-benzo[1,4]oxazin-3-one (30);
8-{2-[2-(3,4,5-trifluoro-phenyl)-1,1-dimethyl-ethylamino]-1-hydroxy-ethyl}-6-hydroxy-4H-benzo[1,4]oxazin-3-one (1.31);
8-{2-[2-(3-methyl-phenyl)-1,1-dimethyl-ethylamino]-1-hydroxy-ethyl}-6-hydroxy-4H-benzo[1,4]oxazin-3-one (1.32) and
8-{2-[2-(3,4-dichloro-phenyl)-1,1-dimethyl-ethylamino]-1-hydroxy-ethyl}-6-hydroxy-4H-benzo[1,4]oxazin-3-one (1.33)
   at least one other active substance 2.

Particularly preferred are, especially, those medicament combinations which contain in addition to one or more, preferably one, compound of general formula 1 selected from the compounds
6-hydroxy-8-{1-hydroxy-2-[2-(4-methoxy-phenyl)-1,1-dimethyl-ethylamino]-ethyl}-4H-benzo[1,4]oxazin-3-one (1.1);
6-hydroxy-8-{1-hydroxy-2-[2-(4-phenoxyethyl-acetate)-1,1-dimethyl-ethylamino]-ethyl}-4H-benzo[1,4]oxazin-3-one (1.2);
6-hydroxy-8-{1-hydroxy-2-[2-(4-phenoxy-acetic acid)-1,1-dimethyl-ethylamino]-ethyl}-4H-benzo[1,4]oxazin-3-one (1.3);
8-{2-[1,1-dimethyl-2-(2,4,6-trimethylphenyl)-ethylamino]-1-hydroxy-ethyl}-6-hydroxy-4H-benzo[1,4]oxazin-3-one (1.4);
6-hydroxy-8-{1-hydroxy-2-[2-(4-hydroxy-phenyl)-1,1-dimethyl-ethylaamino]-ethyl}-4H-benzo[1,4]oxazin-3-one (1.5);
6-hydroxy-8-{1-hydroxy-2-[2-(4-isopropyl-phenyl)-1,1 dimethyl-ethylamino]-ethyl}-4H-benzo[1,4]oxazin-3-one (1.6);
8-{2-[2-(4-ethyl-phenyl)-1,1-dimethyl-ethylamino]-1-hydroxy-ethyl}-6-hydroxy-4H-benzo[1,4]oxazin-3-one (1.7);
8-{2-[2-(4-ethoxy-phenyl)-1,1-dimethyl-ethylamino]-1-hydroxy-ethyl}-6-hydroxy-4H-benzo[1,4]oxazin-3-one (1.12);
4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid (1.14) and
8-{2-[2-(3,4-difluoro-phenyl)-1,1-dimethyl-ethylamino]-1-hydroxy-ethyl}-6-hydroxy-4H-benzo[1,4]oxazin-3-one (1.15),
   at least one other active substance 2.

In the medicament combinations according to the invention the compounds of formula 1 may be present in the form of the individual optical isomers, mixtures of the individual enantiomers or racemates. Particularly preferred are those medicament combinations wherein one or more, preferably one compound of formula 1 is in the form of the enantiomerically pure compounds, preferably in the form of the R-enantiomers. Methods of separating racemates into the various enantiomers are known in the art and may be used to prepare the enantiomerically pure R- or S-enantiomers of the compounds of formula 1 analogously.

In another aspect the present invention relates to medicament combinations which contain the above-mentioned compounds of formula 1 in the form of the acid addition salts with pharmacologically acceptable acids as well as optionally in the form of the solvates and/or hydrates.

By acid addition salts with pharmacologically acceptable acids are meant for example salts selected from the group comprising the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrooxalate, hydrosuccinate, hydrobenzoate and hydro-p-toluenesulphonate, preferably the hydrochloride, hydrobromide, hydrosulphate, hydrophosphate, hydrofumarate and hydromethanesulphonate. Of the above-mentioned acid addition salts, the salts of hydrochloric acid, methanesulphonic acid, benzoic acid and acetic acid are particularly preferred according to the invention.

Preferred medicament combinations contain in addition to one or more, preferably one compound of formula 1 as an additional active substance one or more, preferably one anticholinergic 2a, optionally in combination with pharmaceutically acceptable excipients.

In the medicament combinations according to the invention the anticholinergic 2a is preferably selected from among the tiotropium salts (2a.1), oxitropium salts (2a.2), flutropium salts (a.3 , ipratropium salts (2a.4), glycopyrronium salts (2a.5), trospium salts (2a.6) and the compounds of formulae 2a.7 to 2a.13.

In the above-mentioned salts 2a.1 to 2a.6 the cations tiotropium, oxitropium, flutropium, ipratropium, glycopyrronium and trospium are the pharmacologically active constituents. Explicit references to the above-mentioned cations are indicated by the numerals 2a.1′ to 2a.6′. Each reference to the above-mentioned salts 2a.1 to 2a.6 naturally includes a reference to the corresponding cations tiotropium (2a.1′), oxitropium (2a.2′), flutropium (2a.3′), ipratropium (2a.4′), glycopyrronium (2a.5′) and trospium (2a.6′).

By the salts 2a.1 to 2a.6 are meant according to the invention those compounds which contain in addition to the cations tiotropium (2a.1′), oxitropium (2a.2′), flutropium (2a.3′), ipratropium (2a.4′), glycopyrronium (2a.5′) and trospium (2a.6′) as counter-ion (anion) chloride, bromide, iodide, sulphate, phosphate, methanesulphonate, nitrate, maleate, acetate, citrate, fumarate, tartrate, oxalate, succinate, benzoate or p-toluenesulphonate contain, while the chloride, bromide, iodide, sulphate, methanesulphonate or p-toluenesulphonate are preferred as counter-ions. Of all the salts the chloride, bromide, iodide and methanesulphonate are particularly preferred.

In the case of the trospium salts (2a.6) the chloride is particularly preferred. Of the other salts 2a.1 to 2a.5 the methanesulphonates and bromides are of particular importance. Of particular importance are medicament combinations which contain tiotropium salts (2a.1), oxitropium salts (2a.2) or ipratropium salts (2a.4), while the respective bromides are particularly important according to the invention. Of particular importance is the tiotropium bromide (2a.1). The above-mentioned salts may optionally be present in the medicament combinations according to the invention in the form of their solvates or hydrates, preferably in the form of their hydrates. In the case of tiotropium bromide the medicament combinations according to the invention preferably contain this in the form of the crystalline tiotropium bromide monohydrate, which is known from WO 02/30928. If the tiotropium bromide is used in anhydrous form in the medicament combinations according to the invention, it is preferable to use the anhydrous crystalline tiotropium bromide which is known from WO 03/000265.

Examples of preferred medicament combinations according to the invention of preferred compounds of formula 1 with the above-mentioned anticholinergics 2a.1 to 2a.6 are combinations containing the compounds 1.1 and 2a.1; 1.1 and 2a.2; 1.1 and 2a.3; 1.1 and 2a.4; 1.1 and 2a.5; 1.1 and 2a.6; 1.2 and 2a.1; 1.2 and 2a.2; 1.2 and 2a.3; 1.2 and 2a.4; 1.2 and 2a.5; 1.2 and 2a.6; 1.3 and 2a.1; 1.3 and 2a.2; 1.3 and 2a.3; 1.3 and 2a.4; 1.3 and 2a.5; 1.3 and 2a.6; 1.4 and 2a.1; 1.4 and 2a.2; 1.4 and 2a.3; 1.4 and 2a.4; 1.4 and 2a.5; 1.4 and 2a.6; 1.5 and 2a.1; 1.5 and 2a.2; 1.5 and 2a.3; 1.5 and 2a.4; 1.5 and 2a.5; 1.5 and 2a.6; 1.6 and 2a.1; 1.6 and 2a.2; 1.6 and 2a.3; 1.6 and 2a.4; 1.6 and 2a.5; 1.6 and 2a.6; 1.7 and 2a.1; 1.7 and 2a.2; 1.7 and 2a.3; 1.7 and 2a.4; 1.7 and 2a.5; 1.7 and 2a.6; 1.12 and 2a.1; 1.12 and 2a.2; 1.12 and 2a.3; 1.12 and 2a.4; 1.12 and 2a.5; 1.12 and 2a.6; 1.14 and 2a.1; 1.14 and 2a.2; 1.14 and 2a.3; 1.14 and 2a.4; 1.14 and 2a.5; 1.14 and 2a.6; 1.15 and 2a.1; 1.15 and 2a.2; 1.15 and 2a.3; 1.15 and 2a.4; 1.15 and 2a.5 or 1.15 and 2a.6, in each case optionally in the form of the racemates, enantiomers or diastereomers thereof and optionally in the form of the pharmacologically acceptable acid addition salts, solvates and/or hydrates thereof.

Of the above-mentioned combinations, preferred ones according to the invention are those which contain as compound of formula 1 one of the compounds 1.1, 1.5, 1.6, or 1.12, while those combinations which contain one of the compounds 1.1 or 1.12 are particularly important according to the invention. Of the above-mentioned combinations also preferred according to the invention are those which contain as compound 2a one of the compounds 2a.1, 2a.2 or 2a.4, while those combinations which contain the compound 2a.1 are particularly important according to the invention.

The above-mentioned anticholinergics optionally have chiral carbon centres. In this case the medicament combinations according to the invention may contain the anticholinergics in the form of their enantiomers, mixtures of enantiomers or racemates, while enantiomerically pure anticholinergics are preferably used.

In another preferred embodiment of the present invention the anticholinergics 2a contained in the medicament combinations according to the invention are selected from the salts of formula 2a.7 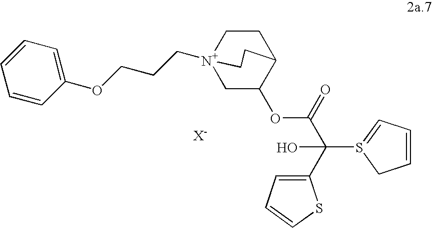
wherein
X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate,
   optionally in the form of the racemates, enantiomers or hydrates thereof.

Preferred medicament combinations contain salts of formula 2a.7, wherein
X⁻ denotes an anion with a single negative charge, preferably an anion selected from among the fluoride, chloride, bromide, methanesulphonate and p-toluenesulphonate, preferably bromide,
   optionally in the form of the racemates, enantiomers or hydrates thereof.

Preferred medicament combinations contain salts of formula 2a.7, wherein
X⁻ denotes an anion with a single negative charge, preferably an anion selected from among the chloride, bromide and methanesulphonate, preferably bromide,
   optionally in the form of the racemates, enantiomers or hydrates thereof.

Particularly preferred medicament combinations contain the compound of formula 2a.7 in the form of the bromide.

Of particular importance are those medicament combinations which contain the enantiomers of formula 2a.7-en 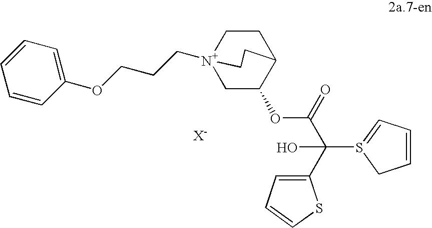
wherein X⁻ may have the above-mentioned meanings.

Examples of novel medicament combinations of preferred compounds of formula 1 with the above-mentioned anticholinergics 2a.7 are combinations containing the compounds 1.1 and 2a.7; 1.1 and 2a.7-en; 1.2 and 2a.7; 1.2 and 2a.7-en; 1.3 and 2a.7; 1.3 and 2a.7-en; 1.4 and 2a.7; 1.4 and 2a.7-en; 1.5 and 2a.7; 1.5 and 2a.7-en; 1.6 and 2a.7; 1.6 and 2a.7-en; 1.7 and 2a.7; 1.7 and 2a.7-en; 1.12 and 2a.7; 1.12 and 2a.7-en; 1.14 and 2a.7; 1.14 and 2a.7-en; 1.15 and 2a.7; 1.15 and 2a.7-en, in each case optionally in the form of the racemates, enantiomers or diastereomers thereof and optionally in the form of the pharmacologically acceptable acid addition salts, solvates and/or hydrates thereof.

Of the above-mentioned combinations preferred ones according to the invention are those which contain as compound of formula 1 one of the compounds 1.1, 1.5, 1.6, or 1.12, while those combinations which contain one of the compounds 1.1 or 1.12 are particularly important according to the invention. Of the above-mentioned combinations also preferred according to the invention are those which contain the compound 2a.7-en as compound 2a.

In another preferred embodiment of the present invention the anticholinergics 2a contained in the medicament combinations according to the invention are selected from the salts of formula 2a.8 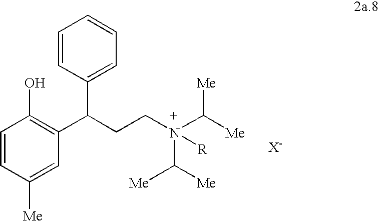
wherein R denotes either methyl (2a.8.1) or ethyl (2a.8.2) and wherein X⁻ may have the above-mentioned meanings. In an alternative embodiment the compound of formula 2a.8 is present in the form of the free base 2a.8-base

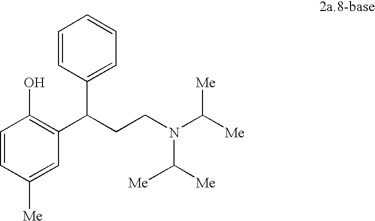

The medicament combinations according to the invention may containthe anticholinergic of formula 2a.8 (or 2a.8-base) in the form of the enantiomers, mixtures of enantiomers or racemates thereof. Preferably the anticholinergics of formula 2a.8 (or 2a.8-base) are present in the form of their R-enantiomers.

Examples of novel medicament combinations of preferred compounds of formula I with the above-mentioned anticholinergics 2a.8 are combinations containing the compounds 1.1 and 2a.8.1; 1.1 and 2a.8.2; 1.2 and 2a.8.1; 1.2 and 2a.8.2; 1.3 and 2a.8.1; 1.3 and 2a.8.2; 1.4 and 2a.8.1; 1.4 and 2a.8.2; 1.5 and 2a.8.1; 1.5 and 2a.8.2; 1.6 and 2a.8.1; 1.6 and 2a.8.2; 1.7 and 2a.8.1; 1.7 and 2a.8.2; 1.12 and 2a.8.1; 1.12 and 2a.8.2; 1.14 and 2a.8.1; 1.14 and 2a.8.2; 1.15 and 2a.8.1; 1.15 and 2a8.2, in each case optionally in the form of the racemates, enantiomers or diastereomers thereof and optionally in the form of the pharmacologically acceptable acid addition salts, solvates and/or hydrates thereof.

Of the above-mentioned combinations, preferred ones according to the invention are those which contain as compound of formula I one of the compounds 1.1, 1.5, 1.6, or 1.12, while those combinations which contain one of the compounds 1.1 or 1.12 are particularly important according to the invention.

In another preferred embodiment of the present invention the anticholinergics 2a contained in the medicament combinations according to the invention are selected from the compounds of formula 2a.9 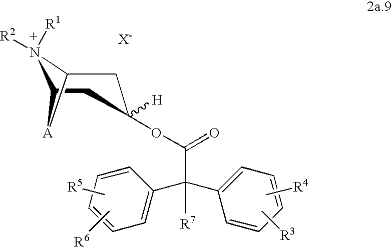
wherein
A denotes a double-bonded group selected from the groups 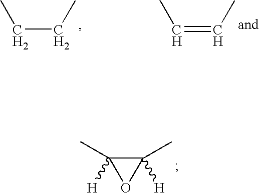
X⁻ denotes one of the above-mentioned anions with a single negative charge, preferably chloride, bromide or methanesulphonate,
R¹ and R² which may be identical or different denote a group selected from methyl, ethyl, n-propyl and iso-propyl, which may optionally be substituted by hydroxy or fluorine, preferably unsubstituted methyl;
R³, R⁴, R⁵ and R⁶, which may be identical or different denote hydrogen, methyl, ethyl, methyloxy, ethyloxy, hydroxy, fluorine, chlorine, bromine, CN, CF₃ or NO₂;
R⁷ denotes hydrogen, methyl, ethyl, methyloxy, ethyloxy, —CH₂—F, —CH₂—CH₂—F, —O—CH₂—F, —O—CH₂—CH₂—F, —CH₂—OH, —CH₂—CH₂—OH, CF₃, —CH₂—OMe, —CH₂—CH₂—OMe, —CH₂—OEt, —CH₂—CH₂—OEt, —O—OMe, —O—COEt, —O—COCF₃, —O—COCF₃, fluorine, chlorine or bromine.

The compounds of formula 2a.9 are known in the art (WO 02/32899).

Within the scope of the medicament combinations according to the invention preferred compounds of formula 2a.9 are those wherein
X⁻ denotes bromide;
R¹ and R² which may be identical or different, denote methyl or ethyl, preferably methyl;
R³, R⁴, R⁵ and R⁶, which may be identical or different, denote hydrogen, methyl, methyloxy, chlorine or fluorine;
R⁷ denotes hydrogen, methyl or fluorine.

Of particular importance are medicament combinations which contain compounds of formula 2a.9, wherein
A denotes a double-bonded group selected from

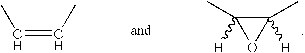

Of particular importance are those medicament combinations which contain in addition to a compound of formula 1 one of the following compounds of formula 2a.9:
tropenol 2,2-diphenylpropionate methobromide (2a.9.1),
scopine 2,2-diphenylpropionate methobromide (2a.9.2),
scopine 2-fluoro-2,2-diphenylacetate methobromide (2a.9.3),
tropenol 2-fluoro-2,2-diphenylacetate methobromide (2a.9.4),;

The compounds of formula 2a.9 may optionally in the form of the enantiomers, mixtures of enantiomers or racemates thereof, as well as optionally in the form of the hydrates and/or solvates thereof.

Examples of novel medicament combinations of preferred compounds of formula I with the above-mentioned anticholinergics 2a.9 are combinations containing the compounds 1.1 and 2a.9.1; 1.1 and 2a.9.2; 1.1 and 2a.9.3; 1.1 and 2a.9.4; 1.2 and 2a.9.1; 1.2 and 2a.9.2; 1.2 and 2a.9.3; 1.2 and 2a.9.4; 1.3 and 2a.9.1; 1.3 and 2a.9.2; 1.3 and 2a.9.3; 1.3 and 2a.9.4;L1.4 and 2a.9.1; 1.4 and 2a.9.2; 1.4 and 2a.9.3; 1.4 and 2a.9.4; 1.5 and 2a.9.1; 1.5 and 2a.9.2; 1.5 and 2a.9.3; 1.5 and 2a.9.4; 1.6 and 2a.9.1; 1.6 and 2a.9.2; 1.6 and 2a.9.3; 1.6 and 2a.9.4; 1.7 and 2a.9.1; 1.7 and 2a.9.2; 1.7 and 2a.9.3; 1.7 and 2a.9.4; 1.12 and 2a.9.1; 1.12 and 2a.9.2; 1.12 and 2a.9.3; 1.12 and 2a.9.4; 1.14 and 2a.9.1; 1.14 and 2a.9.2; 1.14 and 2a.9.3; 1.14 and 2a.9.4; 1.15 and 2a.9.1; 1.15 and 2a.9.2; 1.15 and 2a.9.3; 1.15 and 2a.9.4, in each case optionally in the form of the racemates, enantiomers or diastereomers thereof and optionally in the form of the pharmacologically acceptable acid addition salts, solvates and/or hydrates thereof.

Of the above-mentioned combinations, preferred ones according to the invention are those which contain as compound of formula 1 one of the compounds 1.1, 1.5, 1.6, or 1.12, while those combinations which contain one of the compounds 1.1 or 1.12 are particularly important according to the invention. Of the above-mentioned combinations also preferred according to the invention are those which contain as compound 2a.9 one of the compounds 2a.9.1 or 2a.9.2, while those combinations which contain the compound 2a.9.2 are particularly important according to the invention.

In another preferred embodiment of the present invention the anticholinergics 2a contained in the medicament combinations according to the invention are selected from the compounds of formula 2a.10 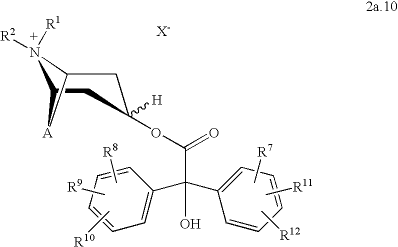
wherein
A, X⁻, R¹ and R² may have the meanings given above and wherein
R⁷, R⁸, R⁹, R¹⁰, R¹¹ and R¹² , which may be identical or different, denote hydrogen, methyl, ethyl, methyloxy, ethyloxy, hydroxy, fluorine, chlorine, bromine, CN, CF₃ or NO₂, while at least one of the groups R⁷, R⁸, R⁹, R¹⁰, R¹¹ and R¹² may not be hydrogen.

The compounds of formula 2a.10 are known in the art (WO 02/32898).

Within the scope of the medicament combinations according to the invention preferred compounds of formula 2a.10 are those wherein
A denotes a double-bonded group selected from 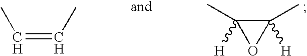
X⁻ bromide;
R¹ and R² which may be identical or different, denote methyl or ethyl, preferably methyl;
R⁷, R⁸, R⁹, R¹⁰, R¹¹ and R ¹², which may be identical or different, denote hydrogen, fluorine, chlorine or bromine, preferably fluorine, while at least one of the groups R⁷, R⁸, R⁹, R¹⁰, R¹¹ and R¹² may not be hydrogen.

Of particular importance are those medicament combinations which contain in addition to a compound of formula 1 one of the following compounds of formula 2a.10:
tropenol 3,3′,4,4′-tetrafluorobenzilate methobromide (2a.10.1),
scopine 3,3′,4,4′-tetrafluorobenzilate methobromide (2a.10.2),
tropenol 4,4′-difluorobenzilate methobromide (2a.10.3),
scopine 4,4′-difluorobenzilate methobromide (2a.10.4),
tropenol 3,3′-difluorobenzilate methobromide (2a.10.5),
scopine 3,3′-difluorobenzilate methobromide (2a.10.6).

The compounds of formula 2a.10 may optionally be presentin the form of the enantiomers, mixtures of enantiomers or racemates thereof, as well as optionally in the form of the hydrates and/or solvates thereof .

Examples of novel medicament combinations of preferred compounds of formula 1 with the above-mentioned anticholinergics 2a.10 are combinations containing the compounds 1.1 and 2a.10.1; 1.1 and 2a.10.2; 1.1 and 2a.10.3; 1.1 and 2a.10.4; 1.1 and 2a.10.5; 1.1 and 2a.10.6; 1.2 and 2a.10.1; 1.2 and 2a.10.2; 1.2 and 2a.10.3; 1.2 and 2a.10.4; 1.2 and 2a.10.5; 1.2 and 2a.10.6; 1.3 and 2a.10.1; 1.3 and 2a.10.2; 1.3 and 2a.10.3; 1.3 and 2a.10.4; 1.3 and 2a.10.5; 1.3 and 2a.10.6; 1.4 and 2a.10.1; 1.4 and 2a.10.2; 1.4 and 2a.10.3; 1.4 and 2a.10.4; 1.4 and 2a.10.5; 1.4 and 2a.10.6; 1.5 and 2a.10.1; 1.5 and 2a.10.2; 1.5 and 2a.10.3; 1.5 and 2a.10.4; 1.5 and 2a.10.5; 1.5 and 2a.10.6; 1.6 and 2a.10.1; 1.6 and 2a.10.2; 1.6 and 2a.10.3; 1.6 and 2a.10.4; 1.6 and 2a.10.5; 1.6 and 2a.10.6; 1.7 and 2a.10.1; 1.7 and 2a.10.2; 1.7 and 2a.10.3; 1.7 and 2a.10.4; 1.7 and 2a.10.5; 1.7 and 2a.10.6; 1.12 and 2a.10.1; 1.12 and 2a.10.2; 1.12 and 2a.10.3; 1.12 and 2a.10.4; 1.12 and 2a.10.5; 1.12 and 2a.10.6; 1.14 and 2a.10.1; 1.14 and 2a.10.2; 1.14 and 2a.10.3; 1.14 and 2a.10.4; 1.14 and 2a.10.5; 1.14 and 2a.10.6; 1.15 and 2a.10.1; 1.15 and 2a.10.2; 1.15 and 2a.10.3; 1.15 and 2a.10.4; 1.15 and 2a.10.5; 1.15 and 2a.10.6, in each case optionally in the form of the racemates, enantiomers or diastereomers thereof and optionally in the form of the pharmacologically acceptable acid addition salts, solvates and/or hydrates thereof.

Of the above-mentioned combinations, preferred ones according to the invention are those which contain as compound of formula 1 one of the compounds 1.1 1.5, 1.6, or 1.12, while those combinations which contain one of the compounds 1.1 or 1.12 are particularly important according to the invention. Of the above-mentioned combinations, also preferred according to the invention are those which contain as compound 2a.10 one of the compounds 2a.10.1, 2a.10.2 , 2a.10.3 or 2a.10.4, while those combinations which contain the compounds 2a.10.1 or 2a.10.2 are particularly important according to the invention.

In another preferred embodiment of the present invention the anticholinergics 2a contained in the medicament combinations according to the invention are selected from the compounds of formula 2a.11 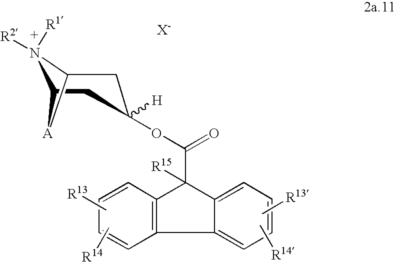
wherein
A and X⁻ may have the meanings given above and wherein
R¹⁵ denotes hydrogen, hydroxy, methyl, ethyl, —CF₃, CHF₂ or fluorine;
R^{1′} and R^{2′} which may be identical or different, denote C₁-C₅-alkyl, which may optionally be substituted by C₃-C₆-cycloalkyl, hydroxy or halogen, or
   R^{1′} and R^{2′} together denote a —C₃-C₅-alkylene bridge;
R¹³, R¹⁴, R^{13′} and R^{14′} which may be identical or different, denote hydrogen, —C₁-C₄-alkyl,
   —C ₁-C₄-alkyloxy, hydroxy, —CF₃, —CHF₂, CN, NO₂ or halogen.

The compounds of formula 2a.11 are known in the art (WO 03/064419).

Within the scope of the medicament combinations according to the invention preferred compounds of formula 2a.11 are those wherein
A denotes a double-bonded group selected from 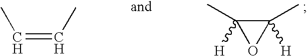
X⁻ denotes an anion selected from chloride, bromide and methanesulphonate, preferably bromide;
R¹⁵ denotes hydroxy, methyl or fluorine, preferably methyl or hydroxy;
R^{1′} and R^{2′} which may be identical or different, denote methyl or ethyl, preferably methyl;
R¹³, R¹⁴, R^{13′} and R^{14′} which may be identical or different, denote hydrogen, —CF₃, —CHF₂ or fluorine, preferably hydrogen or fluorine.

Within the scope of the medicament combinations according to the invention particularly preferred compounds of formula 2a.11 are those wherein
A denotes a double-bonded group selected from 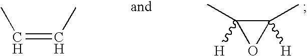
X⁻ denotes bromide;
R¹⁵ denotes hydroxy or methyl, preferably methyl;
R^{1′} and R^{2′} which may be identical or different, denote methyl or ethyl, preferably methyl;
R¹³, R¹⁴, R^{13′} and R^{14′} which may be identical or different, denote hydrogen or fluorine.

Of particular importance are those medicament combinations which contain in addition to a compound of formula 1 one of the following compounds of formula 2a.11
tropenol 9-hydroxy-fluorene-9-carboxylate methobromide (2a.11.1);
tropenol 9-fluoro-fluorene-9-carboxylate methobromide (2a.11.2);
scopine 9-hydroxy-fluorene-9-carboxylate methobromide (2a.11.3);
scopine 9-fluoro-fluorene-9-carboxylate methobromide (2a.11.4);
tropenol 9-methyl-fluorene-9-carboxylate methobromide (2a.11.5);
scopine 9-methyl-fluorene-9-carboxylate methobromide (2a.11.6);

The compounds of formula 2a.11 may optionally be presentin the form of the enantiomers, mixtures of enantiomers or racemates thereof, as well as optionally in the form of the hydrates and/or solvates thereof .

Examples of novel medicament combinations of preferred compounds of formula 1 with the above-mentioned anticholinergics 2a.11 are combinations containing the compounds 1.1 and 2a.11.1; 1.1 and 2a.11.2; 1.1 and 2a.11.3; 1.1 and 2a.11.4; 1.1 and 2a.11.5; 1.1 and 2a.11.6; 1.2 and 2a.11.1; 1.2 and 2a.11.2; 1.2 and 2a.11.3; 1.2 and 2a.11.4; 1.2 and 2a.11.5; 1.2 and 2a.11.6; 1.3 and 2a.11.1; 1.3 and 2a.11.2; 1.3 and 2a.11.3; 1.3 and 2a.11.4; 1.3 and 2a.11.5; 1.3 and 2a.11.6; 1.4 and 2a.11.1; 1.4 and 2a.11.2; 1.4 and 2a.11.3; 1.4 and 2a.11.4; 1.4 and 2a.11.5; 1.4 and 2a.11.6; 1.5 and 2a.11.1; 1.5 and 2a.11.2; 1.5 and 2a.11.3; 1.5 and 2a.11.4; 1.5 and 2a.11.5; 1.5 and 2a.11.6; 1.6 and 2a.11.1; 1.6 and 2a.11.2; 1.6 and 2a.11.3; 1.6 and 2a.11.4; 1.6 and 2a.11.5; 1.6 and 2a.11.6; 1.7 and 2a.11.1; 1.7 and 2a.11.2; 1.7 and 2a.11.3; 1.7 and 2a.11.4; 1.7 and 2a.11.5; 1.7 and 2a.11.6; 1.12 and 2a.11.1; 1.12 and 2a.11.2; 1.12 and 2a.11.3; 1.12 and 2a.11.4; 1.12 and 2a.11.5; 1.12 and 2a.11.6; 1.14 and 2a.11.1; 1.14 and 2a.11.2; 1.14 and 2a.11.3; 1.14 and 2a.11.4; 1.14 and 2a.11.5; 1.14 and 2a.11.6; 1.15 and 2a.11.1; 1.15 and 2a.11.2; 1.15 and 2a.11.3; 1.15 and 2a.11.4; 1.15 and 2a.11.5; 1.15 and 2a.11.6, in each case optionally in the form of the racemates, enantiomers or diastereomers thereof and optionally in the form of the pharmacologically acceptable acid addition salts, solvates and/or hydrates thereof.

Of the above-mentioned combinations, preferred ones according to the invention are those which contain as compound of formula 1 one of the compounds 1.1, 1.5, 1.6, or 1.12, while those combinations which contain one of the compounds 1.1 or 1.12 are particularly important according to the invention. Of the above-mentioned combinations, also preferred according to the invention are those which contain as compound 2a.11 one of the compounds 2a.11.2, 2a.11.4, 2a.11.5 or 2a.11.6, while those combinations which contain the compounds 2a.11.5 or 2a.11.6 are particularly important according to the invention.

In another preferred embodiment of the present invention the anticholinergics 2a contained in the medicament combinations according to the invention are selected from the compounds of formula 2a.12 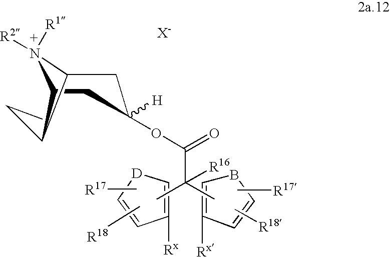
wherein X⁻ may have the meanings given above and wherein
D and B which may be identical or different, are preferably identical and denote O, S, NH, CH₂, CH═CH or
   N(C₁-C₄-alkyl);
R¹⁶ denotes hydrogen, hydroxy, —C₁-C₄-alkyl, —C₁-C₄-alkyloxy, —C₁-C₄-alkylene-halogen, —O—C₁-C₄-alkylene-halogen, —C₁-C₄-alkylene-OH, —CF₃, CHF₂, —C₁-C₄-alkylene-C₁-C₄-alkyloxy, —O—COC₁-C₄-alkyl, —O—COC₁-C₄-alkylene-halogen, —C₁-C₄-alkylene-C₃-C₆-cycloalkyl, —O—COCF₃ or halogen;
R^{1″} and R^{2″} which may be identical or different, denote —C₁-C₅-alkyl, which may optionally be substituted by —C₃-C₆-cycloalkyl, hydroxy or halogen, or
   R^{1″} and R^{2″} together denote a —C₃-C₅-alkylene bridge;
R¹⁷, R¹⁸, R^{17′} and R^{18′}, which may be identical or different, denote hydrogen, —C₁-C₄-alkyl, —C₁-C₄-alkyloxy, hydroxy, —CF₃, —CHF₂, CN, NO₂ or halogen;
R^{x} and R^{x′} which may be identical or different, denote hydrogen, —C₁-C₄-alkyl, —C₁-C₄-alkyloxy, hydroxy, —CF₃, —CHF₂, CN, NO₂ or halogen, or
R^{x} and R^{x′} together denote a single bond or one of the double-bonded groups O, S, NH, CH₂, CH₂—CH₂, N(C₁-C₄-alkyl), CH(C₁-C₄-alkyl) and —C(C₁-C₄-alkyl)₂.

The compounds of formula 2a.12 are known in the art (WO 03/064418).

Within the scope of the medicament combinations according to the invention preferred compounds of formula 2a.12 are those wherein
X⁻ denotes chloride, bromide or methanesulphonate, preferably bromide;
D and B which may be identical or different, are preferably identical and denote O, S, NH or CH═CH;
R¹⁶ denotes hydrogen, hydroxy, —C₁-C₄-alkyl, —C₁-C₄-alkyloxy, —CF₃, —CHF₂, fluorine, chlorine or bromine;
R^{1″} and R^{2″} which may be identical or different, denote C₁-C₄-alkyl, which may optionally be substituted by hydroxy, fluorine, chlorine or bromine, or
R^{1″} and R^{2″} together denote a —C₃-C₄-alkylene bridge;
R¹⁷, R¹⁸, R^{17′} and R^{18″}, which may be identical or different, denote hydrogen, C₁-C₄-alkyl C₁-C₄-alkyloxy, hydroxy, —CF₃, —CHF₂, CN, NO₂, fluorine, chlorine or bromine;
R^{x} and R^{x′} which may be identical or different, denote hydrogen, C₁-C₄-alkyl, C₁-C₄-alkyloxy, hydroxy, —CF₃, —CHF₂, CN, NO₂, fluorine, chlorine or bromine, or
R^{x} and R^{x′} together denote a single bond or a double-bonded group selected from O, S, NH— and CH₂.

Within the scope of the medicament combinations according to the invention particularly preferred compounds of formula 2a.12 are those wherein
X⁻ denotes chloride, bromide, or methanesulphonate, preferably bromide;
D and B which may be identical or different, preferably identical, denote S or CH═CH;
R¹⁶ denotes hydrogen, hydroxy or methyl;
R^{1″} and R^{2″} which may be identical or different, denote methyl or ethyl;
R¹⁷, R¹⁸, R^{17′} and R^{18′}, which may be identical or different, denote hydrogen, —CF₃ or fluorine, preferably hydrogen;
R^{x} and R^{x′} which may be identical or different, denote hydrogen, —CF₃ or fluorine, preferably hydrogen, or
R^{x} and R^{x′} together denote a single bond or —O.

Within the scope of the medicament combinations according to the invention, other particularly preferred compounds of formula 2a.12 are those wherein
X⁻ denotes bromide;
D and B denotes —CH═CH—;
R¹⁶ denotes hydrogen, hydroxy or methyl;
R^{1″} and R^{2″} denotes methyl;
R¹⁷, R¹⁸, R^{17′} and R^{18′}, which may be identical or different, denote hydrogen or fluorine, preferably hydrogen;
R^{x} and R^{x′} which may be identical or different, denote hydrogen or fluorine, preferably hydrogen, or
R^{x} and R^{x′} together denote a single bond or the group —O.

Of particular importance are those medicament combinations which contain in addition to a compound of formula 1 one of the following compounds of formula 2a.12:
cyclopropyltropine benzilate methobromide (2a.12.1);
cyclopropyltropine 2,2-diphenylpropionate methobromide (2a.12.2);
cyclopropyltropine 9-hydroxy-xanthene-9-carboxylate methobromide (2a.12.3);
cyclopropyltropine 9-methyl-fluorene-9-carboxylate methobromide (2a.12.4);
cyclopropyltropine 9-methyl-xanthene-9-carboxylate methobromide (2a.12.5);
cyclopropyltropine 9-hydroxy-fluorene-9-carboxylate methobromide (2a.12.6);
cyclopropyltropine methyl 4,4′-difluorobenzilate methobromide (2a.12.7).

The compounds of formula 2a.12 may optionally be present in the form of the enantiomers, mixtures of enantiomers or racemates thereof, as well as optionally in the form of the hydrates and/or solvates thereof

Examples of novel medicament combinations of preferred compounds of formula 1 with the above-mentioned anticholinergics 2a.12 are combinations containing the compounds 1.1 and 2a.12.1; 1.1 and 2a.12.2; 1.1 and 2a.12.3; 1.1 and 2a.12.4; 1.1 and 2a.12.5; 1.1 and 2a.12.6; 1.1 and 2a.12.7; 1.2 and 2a.12.1; 1.2 and 2a.12.2; 1.2 and 2a.12.3; 1.2 and 2a.12.4; 1.2 and 2a.12.5; 1.2 and 2a.12.6; 1.2 and 2a.12.7; 1.3 and 2a.12.1; 1.3 and 2a.12.2; 1.3 and 2a.12.3; 1.3 and 2a.12.4; 1.3 and 2a.12.5; 1.3 and 2a.12.6; 1.3 and 5 2a.12.7; 1.4 and 2a.12.1; 1.4 and 2a.12.2; 1.4 and 2a.12.3; 1.4 and 2a.12.4; 1.4 and 2a.12.5; 1.4 and 2a.12.6; 1.4 and 2a.12.7; 1.5 and 2a.12.1; 1.5 and 2a.12.2; 1.5 and 2a.12.3; 1.5 and 2a.12.4; 1.5 and 2a.12.5; 1.5 and 2a.12.6; 1.5 and 2a.12.7; 1.6 and 2a.12.1; 1.6 and 2a.12.2; 1.6 and 2a.12.3; 1.6 and 2a.12.4; 1.6 and 2a.12.5; 1.6 and 2a.12.6; 1.6 and 2a.12.7; 1.7 and 2a.12.1; 1.7 and 2a.12.2; 1.7 and 2a.12.3; 1.7 and 10 2a.12.4; 1.7 and 2a.12.5; 1.7 and 2a.12.6; 1.7 and 2a.12.7; 1.12 and 2a.12.1; 1.12 and 2a.12.2; 1.12 and 2a.12.3; 1.12 and 2a.12.4; 1.12 and 2a.12.5; 1.12 and 2a.12.6; 1.12 and 2a.12.7; 1.14 and 2a.12.1; 1.14 and 2a.12.2; 1.14 and 2a.12.3; 1.14 and 2a.12.4; 1.14 and 2a.12.5; 1.14 and 2a.12.6; 1.14 and 2a.12.7; 1.15 and 2a.12.1; 1.15 and 2a.12.2; 1.15 and 2a.12.3; 1.15 and 2a.12.4; 1.15 and 2a.12.5; 1.15 and 2a.12.6; 1.15 and 2a.12.7, in each case optionally in the form of the racemates, enantiomers or diastereomers thereof and optionally in the form of the pharmacologically acceptable acid addition salts, solvates and/or hydrates thereof.

Of the above-mentioned combinations, preferred ones according to the invention are those which contain as compound of formula 1 one of the compounds 1.1 1.5, 1.6, or 1.12, while those combinations which contain one of the compounds 1.1 or 1.12 are particularly important according to the invention. Of the above-mentioned combinations also preferred according to the invention are those which contain as compound 2a.11 one of the compounds 2a.12.1, 2a.12.2, 2a.12.5 or 2a.12.7, while those combinations which contain the compounds 2a.12.1 or 2a.12.2 are particularly important according to the invention.

In another preferred embodiment of the present invention the anticholinergics 2a contained in the medicament combinations according to the invention are selected from the compounds of formula 2a.13 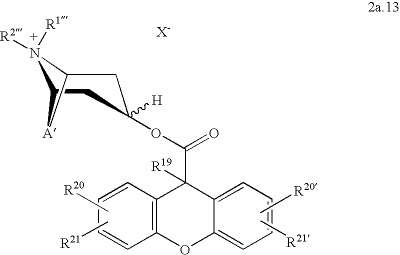
wherein X⁻ may have the meanings given above and wherein
A′ denotes a double-bonded group selected from 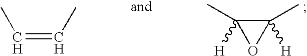
R¹⁹ denotes hydroxy, methyl, hydroxymethyl, ethyl, —CF₃, CHF₂ or fluorine;
R^{1′″} and R^{2′″} which may be identical or different, denote C₁-C₅-alkyl, which may optionally be substituted by C₃-C₆-cycloalkyl, hydroxy or halogen, or
R^{1″′} and R^{2′″} together denote a —C₃-C₅-alkylene bridge;
R²⁰, R²¹, R^{20′} and R^{21′} which may be identical or different, denote hydrogen, —C₁-C₄-alkyl, —C₁-C₄-alkyloxy, hydroxy, —CF₃, —CHF₂, CN, NO₂ or halogen.

The compounds of formula 2a.13 are known in the art (WO 03/064417).

Within the scope of the medicament combinations according to the invention preferred compounds of formula 2a.13 are those wherein
A′ denotes a double-bonded group selected from 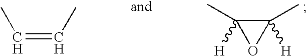
X⁻ denotes chloride, bromide or methanesulphnat, preferably bromide;
R¹⁹ denotes hydroxy or methyl;
R^{1″′ and R}^{2′″} which may be identical or different, denote methyl or ethyl, preferably methyl;
R²⁰, R²¹, R^{20′} and R^{21′} which may be identical or different, denote hydrogen, —CF₃, —CHF₂ or fluorine, preferably hydrogen or fluorine.

Within the scope of the medicament combinations according to the invention particularly preferred compounds of formula 2a.13 are those wherein
A′ denotes a double-bonded group selected from 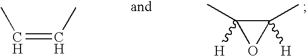
X⁻ denotes bromide;
R¹⁹ denotes hydroxy or methyl, preferably methyl;
R^{1′″} and R^{2′″} which may be identical or different, denote methyl or ethyl, preferably methyl;
R³, R⁴, R^{3′} and R^{4′} which may be identical or different, denote hydrogen or fluorine.

Of particular importance are those medicament combinations which contain in addition to a compound of formula 1 one of the following compounds of formula 2a.13:
tropenol 9-hydroxy-xanthene-9-carboxylate methobromide (2a.13.1);
scopine 9-hydroxy-xanthene-9-carboxylate methobromide (2a.13.2);
tropenol 9-methyl-xanthene-9-carboxylate methobromide (2a.13.3);
scopine 9-methyl-xanthene-9-carboxylate methobromide (2a.1 3.4);
tropenol 9-ethyl-xanthene-9-carboxylate methobromide (2a.13.5);
tropenol 9-difluoromethyl-xanthene-9-carboxylate methobromide (2a.13.6);
scopine 9-hydroxymethyl-xanthene-9-carboxylate methobromide (2a.13.7).

The compounds of formula 2a.13 may optionally be present in the form of the enantiomers, mixtures of enantiomers or racemates thereof, as well as optionally in the form of the hydrates and/or solvates thereof .

Examples of novel medicament combinations of preferred compounds of formula 1 with the above-mentioned anticholinergics 2a.13 are combinations containing the compounds 1.1 and 2a.13.1; 1.1 and 2a.13.2; 1.1 and 2a.13.3; 1.1 and 2a.13.4; 1.1 and 2a.13.5; 1.1 and 2a.13.6; 1.1 and 2a.13.7; 1.2 and 2a.13.1; 1.2 and 2a.13.2; 1.2 and 2a.13.3; 1.2 and 2a.13.4; 1.2 and 2a.13.5; 1.2 and 2a.13.6; 1.2 and 2a.13.7; 1.3 and 2a.13.1; 1.3 and 2a.13.2; 1.3 and 2a.13.3; 1.3 and 2a.13.4; 1.3 and 2a.13.5; 1.3 and 2a.13.6; 1.3 and 2a.13.7; 1.4 and 2a.13.1; 1.4 and 2a.13.2; 1.4 and 2a.13.3; 1.4 and 2a.13.4; 1.4 and 2a.13.5; 1.4 and 2a.13.6; 1.4 and 2a.13.7; 1.5 and 2a.13.1; 1.5 and 2a.13.2; 1.5 and 2a.13.3; 1.5 and 2a.13.4; 1.5 and 2a.13.5; 1.5 and 2a.13.6; 1.5 and 2a.13.7; 1.6 and 2a.13.1; 1.6 and 2a.13.2; 1.6 and 2a.13.3; 1.6 and 2a.13.4; 1.6 and 2a.13.5; 1.6 and 2a.13.6; 1.6 and 2a.13.7; 1.7 and 2a.13.1; 1.7 and 2a.13.2; 1.7 and 2a.13.3; 1.7 and 2a.13.4; 1.7 and 2a.13.5; 1.7 and 2a.13.6; 1.7 and 2a.13.7; 1.12 and 2a.13.1; 1.12 and 2a.13.2; 1.12 and 2a.13.3; 1.12 and 2a.13.4; 1.12 and 2a.13.5; 1.12 and 2a.13.6; 1.12 and 2a.13.7; 1.14 and 2a.13.1; 1.14 and 2a.13.2; 1.14 and 2a.13.3; 1.14 and 2a.13.4; 1.14 and 2a.13.5; 1.14 and 2a.13.6; 1.14 and 2a.13.7; 1.15 and 2a.13.1; 1.15 and 2a.13.2; 1.15 and 2a.13.3; 1.15 and 2a.13.4; 1.15 and 2a.13.5; 1.15 and 2a.13.6; 1.15 and 2a.13.7, in each case optionally in the form of the racemates, enantiomers or diastereomers thereof and optionally in the form of the pharmacologically acceptable acid addition salts, solvates and/or hydrates thereof.

Of the above-mentioned combinations, preferred ones according to the invention are those which contain as compound of formula 1 one of the compounds 1.1, 1.5, 1.6, or 1.12, while those combinations which contain one of the compounds 1.1 or 1.12 are particularly important according to the invention. Of the above-mentioned combinations also preferred according to the invention are those which contain as compound 2a.11 one of the compounds 2a.13.2, 2a.13.3, 2a.13.4 or 2a.13.5, while those combinations which contain the compounds 2a.13.3 or 2a.13.4 are particularly important according to the invention.

Within the scope of the present invention any reference to anticholinergics 1′ is to be taken as a reference to the pharmacologically active cations of the various salts . These cations are tiotropium (2a.1′), oxitropium (2a.2′), flutropium (2a.3′), ipratropium (2a.4′), glycopyrronium (2a.5′), trospium (2a.6′) and the cations shown below:

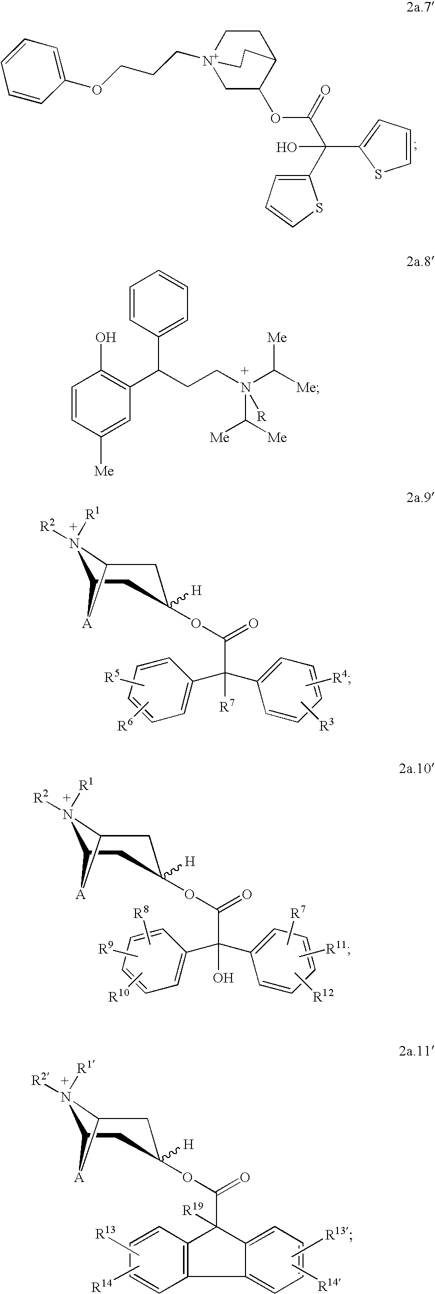

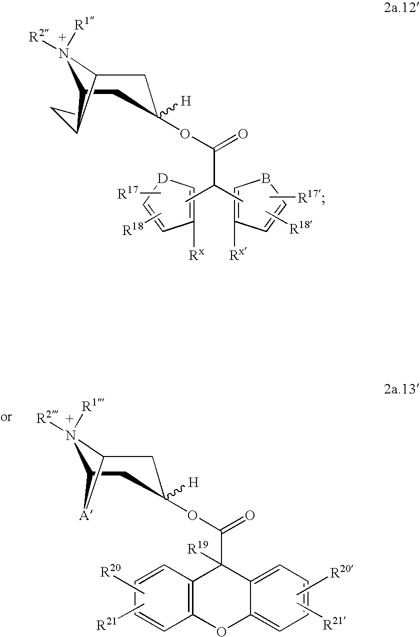

Other preferred medicament combinations according to the invention contain as an additional active substance, in addition to one or more, preferably one compound of formula 1, one or more, preferably one, PDE IV-inhibitor 2b, optionally in combination with pharmaceutically acceptable excipients.

In such medicament combinations the PDE IV-inhibitor 2b is preferably selected from among enprofyllin, theophyllin, roflumilast, ariflo (Cilomilast), CP-325,366, BY343, D-4396 (Sch-351591), AWD-12-281 (GW-842470), N-(3,5-dichloro-1-oxo-pyridin-4-yl)-4-difluoromethoxy-3-cyclopropylmethoxybenzamide, NCS-613, pumafentine, (−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide, (R)-(+)-1-(4-bromobenzyl)-4-[(3-cyclopentyloxy)-4-methoxyphenyl]-2-pyrrolidone, 3-(cyclopentyloxy-4-methoxyphenyl)-1-(4-N′-[N-2-cyano-S-methyl-isothioureido]benzyl)-2-pyrrolidone, cis[4-cyano-4-(3-cyclopentyloxy-4-methoxyphenyl)cyclohexane-1-carboxylic acid], 2-carbomethoxy-4-cyano-4-(3-cyclopropylmethoxy-4-difluoromethoxyphenyl)cyclohexan-1-one, cis[4-cyano-4-(3-cyclopropylmethoxy-4-difluoromethoxyphenyl)cyclohexan-1-ol], (R)-(+)-ethyl[4-(3-cyclopentyloxy-4-methoxyphenyl)pyrrolidin-2-ylidene]acetate, (S)-(−)-ethyl[4-(3-cyclopentyloxy-4-methoxyphenyl)pyrrolidin-2-ylidene]acetate, CDP840, Bay-198004, D-4418, PD-168787, T-440, T-2585, arofyllin, atizoram, V-11294A, Cl-1018, CDC-801, CDC-3052, D-22888, YM-58997, Z-15370, 9-cyclopentyl-5,6-dihydro-7-ethyl-3-(2-thienyl)-9H-pyrazolo[3,4-c]-1,2,4-triazolo[4,3-a]pyridine and 9-cyclopentyl-5,6-dihydro-7-ethyl-3-(tert-butyl)-9H-pyrazolo[3,4-c]-1,2,4-triazolo[4,3-a]pyridine, optionally in the form of the racemates, enantiomers or diastereomers thereof and optionally in the form of the pharmacologically acceptable acid addition salts, solvates and/or hydrates thereof.

In particularly preferred medicament combinations the PDE IV-inhibitor 2b is selected from the group comprising enprofyllin (2b.1), roflumilast (2b2), ariflo (cilomilast) (2b.3), AWD-12-281 (GW-842470) (2b.4), N-(3,5-dichloro-1-oxo-pyridin-4-yl)-4-difluoromethoxy-3-cyclopropylmethoxybenzamide (2b.5), T-440 (2b.6), T-2585 (2b.7), arofyllin (2b.8), cis[4-cyano-4-(3-cyclopentyloxy-4-methoxyphenyl)cyclohexane-1-carboxylic acid] (2b.9), 2-carbomethoxy-4-cyano-4-(3-cyclopropylmethoxy-4-difluoromethoxyphenyl)cyclohexan-1-one (2b.10), cis[4-cyano-4-(3-cyclopropylmethoxy-4-difluoromethoxyphenyl)cyclohexan-1-ol] (2b.11), PD-168787 (2b.12), atizoram (2b.13), V-11294A (2b.14), Cl-1018 (2b.15), CDC-801 (2b.16), D-22888 (2b.17), YM-58997 (2b.18), Z-15370 (2b.19), 9-cyclopentyl-5,6-dihydro-7-ethyl-3-(2-thienyl)-9H-pyrazolo[3,4-c]-1,2,4-triazolo[4,3-a]pyridine (2b.20) and 9-cyclopentyl-5,6-dihydro-7-ethyl-3-(tert-butyl)-9H-pyrazolo[3,4-c]-1,2,4-triazolo[4,3-a]pyridine (2b.21), optionally in the form of the racemates, enantiomers or diastereomers thereof and optionally in the form of the pharmacologically acceptable acid addition salts, solvates and/or hydrates thereof.

In particularly preferred medicament combinations the PDE IV-inhibitor 2b is selected from the group comprising roflumilast (2b2 , ariflo (cilomilast) (2b.3), AWD-12-281 (GW-842470) (2b.4), arofyllin (2b.8), 2-carbomethoxy-4-cyano-4-(3-cyclopropylmethoxy-4-difluoromethoxyphenyl)cyclohexan-1-one (2b.10), cis[4-cyano-4-(3-cyclopropylmethoxy-4-difluoromethoxyphenyl)cyclohexan-1-ol] (2b.11), atizoram (2b.13), Z-15370 (2b.19), 9-cyclopentyl-5,6-dihydro-7-ethyl-3-(2-thienyl)-9H-pyrazolo[3,4-c]-1,2,4-triazolo[4,3-a]pyridine (2b.20) and 9-cyclopentyl-5,6-dihydro-7-ethyl-3-(tert-butyl)-9H-pyrazolo[3,4-c]-1,2,4-triazolo[4,3-a]pyridine (2b.21), while roflumilast (.2), Z-15370 (2b.19) and AWD-12-281 (.4 are of particular significance, optionally in the form of the racemates, enantiomers or diastereomers thereof and optionally in the form of the pharmacologically acceptable acid addition salts, solvates and/or hydrates thereof.

By the acid addition salts with pharmacologically acceptable acids which the compounds 2b may possibly be capable of forming are meant for example salts selected from the group comprising the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofuimarate, hydrotartrate, hydrooxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Examples of novel preferred medicament combinations of preferred compounds of formula 1 with the above-mentioned PDE IV inhibitors 2b are combinations containing the compounds 1.1 and 2b.1; 1.1 and 2b.2; 1.1 and 2b.3; 1.1 and 2b.4; 1.1 and 2b.5; 1.1 and 2b.6; 1.1 and 2b.7; 1.1 and .; 1.1 and 2b.9; 1.1 and 2b.10; 1.1 and 2b.11; 1.1 and 2b.12; 1.1 and 2b.13; 1.1 and 2b.14; 1.1 and 2b.15; 1.1 and 2b.16; 1.1 and 2b.17; 1.1 and 2b.18; 1.1 and 2b.19; 1.1 and 2b.20; 1.1 and 2b.21; 1.2 and 2b.1; 1.2 and 2b.2; 1.2 and 2b.3; 1.2 and 2b.4-;1.2 and 2b.5; 1.2 and 2b.6; 1.2 and 2b.7; 1.2 and 2b.8; 1.2 and 2b.9; 1.2 and 2b.10; 1.2 and 2b.11; 1.2 and 2b.12; 1.2 and 2b.13; 1.2 and 2b.14; 1.2 and 2b.15; 1.2 and 2b.16; 1.2 and 2b.17; 1.2 and 2b.18; 1.2 and 2b.19; 1.2 and 2b.20; 1.2 and 2b.21; 1.3 and 2b.1; 1.3 and 2b.2; 1.3 and 2b.3; 1.3 and 2b.4; 1.3 and 2b.5; 1.3 and 2b.6; 1.3 and 2b.7; 1.3 and 2b.8; 1.3 and 2b.9; 1.3 and 2b.10; 1.3 and 2b.11; 1.3 and 2b.12; 1.3 and 2b.13; 1.3 and 2b.14; 12b.15; 1.3 and 2b.16; 1.3 and 2b.17; 1.3 and 2b.18; 1.3 and 2b.19; 1.3 and 2b.20; 1.3 and 2b.21; 1.4 and 2b.1; 1.4 and 2b.2; 1.4 and 2b.3; 1.4 and 2b.4; 1.4 and 2b.5; 1.4 and 2b.6; 1.4 and 2b.7; 1.4 and 2b.8; 1.4 and 2b.9; 1.4 and 2b.10; 1.4 and 2b.11; 1.4 and 2b.12; 1.4 and 2b.13; 1.4 and 2b.14; 1.4 and 2b.15; 1.4 and 2b.16; 1.4 and 2b.17; 1.4 and 2b.18; 1.4 and 2b.19; 1.4 and 2b.20; 1.4 and 2b.21; 1.5 and 2b.1; 1.5 and 2b.2; 1.5 and 2b.3; 1.5 and 2b.4; 1.5 and 2b.5; 1.5 and 2b.6; 1.5 and 2b.7; 1.5 and 2b.8; 1.5 and 2b.9; 1.5 and 2b.10; 1.5 and 2b.11; 1.5 and 2b.12; 1.5 and 2b.13; 1.5 and 2b.14; 1.5 and 2b.15; 1.5 and 2b.16; 1.5 and 2b.17; 1.5 and 2b.18; 1.5 and 2b.19; 1.5 and 2b.20; 1.5 and 2b.21; 1.6 and 2b.1; 1.6 and 2b.2; 1.6 and 2b.3; 1.6 and 2b.4; 1.6 and 2b.5; 1.6 and 2b.6; 1.6 and 2b.7; 1.6 and 2b.8; 1.6 and 2b.9; 1.6 and 2b.10; 1.6 and 2b.11; 1.6 and 2b.12; 1.6 and 2b.13; 1.6 and 2b.14; 1.6 and 2b.15; 1.6 and 2b.16; 1.6 and 2b.17; 1.6 and 2b.18; 1.6 and 2b.19; 1.6 and 2b.20; 1.6 and 2b.21; 1.7 and 2b.1; 1.7 and 2b.2; 1.7 and 2b.3; 1.7 and 2b.4; 1.7 and 2b.5; 1.7 and 2b.6; 1.7 and 2b.7; 1.7 and 2b.8; 1.7 and 2b.9; .7and 2b.10; 1.7 and 2b.11; 1.7 and 2b.12; 1.7 and 2b.13; 1.7 and 2b.14; 1.7 and 2b.15; 1.7 and 2b.16; 1.7 and 2b.17; 1.7 and 2b.18; 1.7 and 2b.19; 1.7 and 2b.20; 1.7 and 2b.21; 1.12 and 2b.1; 1.12 and 2b.2; 1.12 and 2b.3; 1.12 and 2b.4; 1.12 and 2b.5; 1.12 and 2b.6; 1.12 and 2b.7; 1.12 and 2b.8; 1.12 and 2b.9; 1.12 and 2b.10; 1.12 and 2b.11; 1.12 and 2b.12; 1.12 and 2b.13; 1.12 and 2b.14; 1.12 and 2b.15; 1.12 and 2b.16; 1.12 and 2b.17; 1.12 and 2b.18; 1.12 and 2b.19; 1.12 and 2b.20; 1.12 and 2b.21; 1.14 and 2b.1; 1.14 and 2b.2; 1.14 and 2b.3; 1.14 and 2b.4; 1.14 and 2b.5; 1.14 and 2b.6; 1.14 and 2b.7; 1.14 and 2b.8; 1.14 and 2b.9; 1.14 and 2b.10; 1.14 and 2b.11; 1.14 and 2b.12; 1.14 and 2b.13; 1.14 and 2b.14; 1.14 and 2b.15; 1.14 and 2b.16; 1.14 and 2b.17; 1.14 and 2b.18; 1.14 and 2b.19; 1.14 and 2b.20; 1.14 and 2b.21; 1.15 and 2b.1; 1.15 and 2b.2; 1.15 and 2b.3; 1.15 and 2b.4; 1.15 and 2b.5; 1.15 and 2b.6; 1.15 and 2b.7; 1.15 and 2b.8; 1.15 and 2b.9; 1.15 and 2b.10; 1.15 and 2b.11; 1.15 and 2b.12; 1.15 and 2b.13; 1.15 and 2b.14; 1.15 and 2b.15; 1.15 and 2b.16; 1.15 and 2b.17; 1.15 and 2b.18; 1.15 and 2b.19; 1.15 and 2b.20 or 1.15 and 2b.21, in each case optionally in the form of the racemates, enantiomers or diastereomers thereof and optionally in the form of the pharmacologically acceptable acid addition salts, solvates and/or hydrates thereof, in each case optionally in the form of the racemates, enantiomers or diastereomers thereof and optionally in the form of the pharmacologically acceptable acid addition salts, solvates and/or hydrates thereof.

Of the above-mentioned combinations, preferred ones according to the invention are those which contain as compound of formula 1 one of the compounds 1.1, 1.5, 1.6, or 1.12, while those combinations which contain one of the compounds 1.1 or 1.12 are particularly important according to the invention. Of the above-mentioned combinations also preferred according to the invention are those which contain as compound 2b one of the compounds 2b.2, 2b.3, 2b.4, 2b.8, 2b.10, 2b.11, 2b.13, 2b.19, 2b.20 or 2 , while those combinations which contain one of the compounds 2b.2, 2b.4 or 2b.19 are particularly important according to the invention.

Other preferred medicament combinations according to the invention contain as an additional active substance, in addition to one or more, preferably one compound of formula 1, one or more, preferably one steroid 2c, optionally in combination with pharmaceutically acceptable excipients.

In such medicament combinations the steroid 2c is preferably selected from among prednisolone (2c.1, prednisone (c.2), butixocortpropionate (2c.3), RPR-106541 (2c.4), flunisolide (2c.5, beclomethasone (2c.6, triamcinolone (2c.7), budesonide (2c.8, fluticasone (2.9), mometasone (2c.10), ciclesonide (2c.11), rofleponide (2c.12), ST-126 (2c.13), dexamethasone (2c.14), (S)-fluoromethyl 6α,9α-difluoro-17α-[(2-furanylcarbonyl)oxy]-11β-hydroxy-16α-methyl-3-oxo-androsta-1,4-diene-17β-carbothionate (2c. 15), (S)-(2-oxo-tetrahydro-furan-3S-yl)6α,9α-difluoro-11β-hydroxy-16α-methyl-3-oxo-17α-propionyloxy-androsta-1,4-diene-17β-carbothionate (2c.16) and etiprednol-dichloroacetate (BNP-166, 2c.17), optionally in the form of the racemates, enantiomers or diastereomers thereof and optionally in the form of the salts and derivatives thereof, the solvates and/or hydrates thereof

In particularly preferred medicament combinations the steroid 2c is selected from the group comprising flunisolide (2c.5) , beclomethasone (2c.6), triamcinolone (2c.7) , budesonide (2c.8), fluticasone (2c.9), mometasone (2c.10), ciclesonide (2c.11), rofleponide (2c.12), ST-126 (2c.13), dexamethasone (2c.14), (S)-fluoromethyl 6α,9α-difluoro-17α-[(2-furanylcarbonyl)oxy]-11β-hydroxy-16α-methyl-3-oxo-androsta-1,4-diene-17β-carbothionate (2c.15), (S)-(2-oxo-tetrahydro-furan-3S-yl)6α,9α-difluoro-11β-hydroxy-16α-methyl-3-oxo-17α-propionyloxy-androsta-1,4-diene-17β-carbothionate (2c.16) and etiprednol-dichloroacetate (2c.17), optionally in the form of the racemates, enantiomers or diastereomers thereof and optionally in the form of the salts and derivatives thereof, the solvates and/or hydrates thereof.

In particularly preferred medicament combinations the steroid 2c is selected from the group comprising budesonide (2.8, fluticasone (2.9), mometasone (2c.10), ciclesonide (2c.11), (S)-fluoromethyl 6α,9α-difluoro-17α-[(2-furanylcarbonyl)oxy]-11β-hydroxy-16α-methyl-3-oxo-androsta-1,4-diene-17β-carbothionate (2c.15) and etiprednol-dichloroacetate (2c.17), optionally in the form of the racemates, enantiomers or diastereomers thereof and optionally in the form of the salts and derivatives thereof, the solvates and/or hydrates thereof.

Any reference to steroids 2c includes a reference to any salts or derivatives, hydrates or solvates thereof which may exist. Examples of possible salts and derivatives of the steroids 2c may be: alkali metal salts, such as for example sodium or potassium salts, sulphobenzoates, phosphates, isonicotinates, acetates, propionates, dihydrogen phosphates, palmitates, pivalates or furoates.

Examples of novel preferred medicament combinations of preferred compounds of formula 1 with the above-mentioned steroids 2c are combinations containing the compounds 1.1 and 2c.1; 1.1 and 2c.2; 1.1 and 2c.3; 1.1 and 2c.4; 1.1 and 2c.5; 1.1 and 2c.6;1. and 2c.7; 1.1 and 2c.8; 1.1 and 2c.9; 1.1 and 2c.10; 1.1 and 2c.11; 1.1 and 2c.12; 1.1 and 2c.13; 1.1 and 2c.14; 1.1 and 2c.15; 1.1 and 2c.16; 1.1 and 2c.17; 1.2 and 2c.1; 1.2 and 2c.2; 1.2 and 2c.3; 1.2 and 2c.4; 1.2 and 2c.5; 1.2 and 2c.6; 1.2 and 2c.7; 1.2 and 2c.8; 1.2 and 2c.9; 1.2 and 2c.10; 1.2 and 2c.11; 1.2 and 2c.12; 1.2 and 2c.13; 1.2 and 2c.14; 1.2 and 2c.15; 1.2 and 2c.16; 1.2 and 2c.17; 1.3 and 2c.1; 1.3 and 2c.2; 1.3 and 2c.3; 1.3 and 2c.4; 1.3 and 2c.5; 1.3 and 2c.6; 1.3 and 2c.7; 1.3 and 2c.8; 1.3 and 2c.9; 1.3 and 2c.10; 1.3 and 2c.11; 1.3 and 2c.12; 1.3 and 2c.13; 1.3 and 2c.14; 1.3 and 2c.15; 1.3 and 2c.16; 1.3 and 2c.17; 1.4 and 2c.1; 1.4 and 2c.2; 1.4 and 2c.3; 1.4 and 2c.4; 1.4 and 2c.5; 1.4 and 2c.6; 1.4 and 2c.7; 1.4 and 2c.8; 1.4 and 2c.9; 1.4 and 2c.10; 1.4 and 2c.11; 1.4 and 2c.12; 1.4 and 2c.13; 1.4 and 2c.14; 1.4 and 2c.15; 1.4 and 2c.16; 1.4 and 2c.17; 1.5 and 2c.1; 1.5 and 2c.2; 1.5 and 2c.3; 1.5 and 2c.4; 1.5 and 2c.5; 1.5 and 2c.6; 1.5 and 2c.7; 1.5 and 2c.8; 1.5 and 2c.9; 1.5 and 2c.10; 1.5 and 2c.11; 1.5 and 2c.12; 1.S and 2c.13; 1.5 and 2c.14; 1.5 and 2c.15; 1.5 and 2c.16; 1.5 and 2c.17; 1.6 and 2c.1; 1.6 and 2c.2; 1.6 and 2c.3; 1.6 and 2c.4; 1.6 and 2c.5; 1.6 and 2c.6; 1.6 and 2c.7; 1.6 and 2c.8; 1.6 and 2c.9; 1.6 and 2c.10; 1.6 and 2c.11; 1.6 and 2c.12; 1.6 and 2c.13; 1.6 and 2c.14; 1.6 and 2c.15; 1.6 and 2c.16; 1.6 and 2c.17; 1.7 and 2c.1; 1.7 and 2c.2; 1.7 and 2c.3; 1.7 and 2c.4; 1.7 and 2c.5; 1.7 and 2c.6; 1.7 and 2c.7; 1.7 and 2c.8; 1.7 and 2c.9; 1.7 and 2c.10; 1.7 and 2c.11; 1.7 and 2c.12; 1.7 and 2c.13; 1.7 and 2c.14; 1.7 and 2c.15; 1.7 and 2c.16; 1.7 and 2c.17; 1.12 and 2c.1; 1.12 and 2c.2; 1.12 and 2c.3; 1.12 and 2c.4; 1.12 and 2c.5; 1.12 and 2c.6; 1.12 and 2c.7; 1.12 and 2c.8; 1.12 and 2c.9; 1.12 and 2c.10; 1.12 and 2c.11; 1.12 and 2c.12; 1.12 and 2c.13; 1.12 and 2c.14; 1.12 and 2c.15; 1.12 and 2c.16; 1.12 and 2c.17; 1.14 and 2c.1; 1.14 and 2c.2; 1.14 and 2c.3; 1.14 and 2c.4; 1.14 and 2c.5; 1.14 and 2c.6; 1.14 and 2c.7; 1.14 and 2c.8; 1.14 and 2c.9; 1.14 and 2c.10; 1.14 and 2c.11; 1.14 and 2c.12; 1.14 and 2c.13; 1.14 and 2c.14; 1.14 and 2c.15; 1.14 and 2c.16; 1.14 and 2c.17; 1.15 and 2c.1; 1.15 and 2c.2; 1.15 and 2c.3; 1.15 and 2c.4; 1.15 and 2c.5; 1.15 and 2c.6; 1.15 and 2c.7; 1.15 and 2c.8; 1.15 and 2c.9; 1.15 and 2c.10; 1.15 and 2c.11; 1.15 and 2c.12; 1.15 and 2c.13; 1.15 and 2c.14; 1.15 and 2c.15, 1.15 and 2c.16 or 1.15 and 2c.17, in each case optionally in the form of the racemates, enantiomers or diastereomers thereof and optionally in the form of the pharmacologically acceptable acid addition salts, solvates and/or hydrates thereof.

Of the above-mentioned combinations, preferred ones according to the invention are those which contain as compound of formula 1 one of the compounds 1.1, 1.5, 1.6, or 1.12, while those combinations which contain one of the compounds 1.1 or 1.12 are particularly important according to the invention. Of the above-mentioned combinations also preferred according to the invention are those which contain as compound 2c one of the compounds 2c.5, 2c.6, 2c.7, 2c.8, 2c.9, 2c.10, 2c.11, 2c.12, 2c.13, 2c.14, 2c.15, 2c.16 or 2c.17 , while those combinations which contain one of the compounds 2c.8, 2c.9,.2c.10, 2c.112c.15 or 2c.17 are particularly important according to the invention.

Other preferred medicament combinations according to the invention contain, as an additional active substance, in addition to one or more, preferably one compound of formula 1, one or more, preferably one, LTD4 antagonist 2d, optionally in combination with pharmaceutically acceptable excipients.

In such medicament combinations the LTD4 antagonist 2d is preferably selected from among montelukast (2d.1) , 1-(((R)-(3-(2-(6,7-difluoro-2-quinolinyl)ethenyl)phenyl)-3-(2-(2-hydroxy-2-propyl)phenyl)thio)methylcyclopropane-acetic acid (2d.2), 1-(((1(R)-3(3-(2-(2,3-dichlorothieno[3,2-b]pyridin-5-yI)-(E)-ethenyl)phenyl)-3-(2-(1-hydroxy-1-methylethyl)phenyl)propyl)thio)methyl)cyclopropanacetic acid (2d.3), pranlukast (2d.4), zafirlukast (2d.5), [2-[[2-(4-tert-butyl-2-thiazolyl)-5-benzofuranyl]oxymethyl]-phenyl]acetic acid (2d.6), MCC-847 (ZD-3523) (2d.7), MN-001 (2d.8), MEN-91507 (LM-1507) (2d.9), VUF-5078 (2d.10), VUF-K-8707 (2d.11) and L-733321 (2d.12), optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

In preferred medicament combinations the LTD4 antagonist 2d is selected from the group comprising montelukast (2d.1), pranlukast (2d.4), zafirlukast (2d.5), MCC-847 (ZD-3523) (2.7, MN-001 (2d.8), MEN-91507 (LM-1507) (2d.9), VUF-5078 (2d.10), VUF-K-8707 (2d.11) and L-733321 (2d.12), optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

In particularly preferred medicament combinations the LTD4 antagonist 2d is selected from the group comprising montelukast (2d.1), pranlukast (2d.4), zafirlukast (2d.5), MCC-847 (ZD-3523) (2d.7), MN-001 (2d.8) and MEN-91507 (LM-1507) (2d.9), while montelukast (2d.1), pranlukast (2d.4) and zafirlukast (2d.5) are particularly preferred, optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

By the acid addition salts with pharmacologically acceptable acids which the compounds 2d may possibly be capable of forming are meant for example salts selected from the group comprising the hydrochloride, hydrobromide, hydroiodide, hydrosulphate, hydrophosphate, hydromethanesulphonate, hydronitrate, hydromaleate, hydroacetate, hydrobenzoate, hydrocitrate, hydroftimarate, hydrotartrate, hydrooxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Examples of possible salts and derivatives which the compounds 2d may possibly be capable of forming include for example: alkali metal salts, such as for example sodium or potassium salts, alkaline earth metal salts, sulphobenzoates, phosphates, isonicotinates, acetates, propionates, dihydrogen phosphates, palmitates, pivalates or furoates.

Examples of novel preferred medicament combinations of preferred compounds of formula 1 with the above-mentioned LTD4-antagonists 2d are combinations containing the compounds 1.1 and 2d.1; 1.1 and 2d.2; 1.1 and 2d.3; 1.1 and 2d.4; 1.1 and 2d.5; 1.1 and 2d.6; 1.1 and 2d.7; 1.1 and 2d.8; 1.1 and 2d.9; 1.1 and 2d.10; 1.1 and 2d.11; 1.1 and 2d.12; 1.2 and 2d.1; 1.2 and 2d.2; 1.2 and 2d.3; 1.2 and 2d.4; 1.2 and 2 d .5; 2d.6; 1.2 and 2d.7; 1.2 and 2d.8; 1.2 and 2d.9; 1.2 and 2d.10; 1.2 and 2d.11; 1.2 and 2d.12; 1.3 and 2d.1; 1.3 and 2d.2; 1.3 and 2d.3; 1.3 and 2d.4; 1.3 and 2d.5; 1.3 and 2d.6; 1.3 and 2d.7; 1.3 and 2d.8; 1.3 and 2d.9; 1.3 and 2d.10; 1.3 and 2d.11; 1.3 and 2d.12; 1.4 and 2d.1; 1.4 and 2d.2; 1.4 and 2d.3; 1.4 and 2d.4; 1.4 and 2d.5; 1.4 and 2d.6; 1.4 and 2d.7; 1.4and2d.8;1.4and2d.9;1.4and2d.10;1.4and2d.11;1.4and2d.12; 1.5 and d.1; 1.5 and 2d.2; 1.5 and 2d.3; 1.5 and 2d.4; 1.5 and 2d.5; 1.5 and 2d.6; 1.5 and 2d.7; 1.5 and 2d.8; 1.5 and 2d.9-1.5 and 2d.10; 1.5 and 2d.11; 1.5 and 2d.12; 1.6 and 2d.1; 1.6 and 2d.2; 1.6 and 2d.3; 1.6 and 2d.4; 1.6 and 2d.5; 1.6 and 2d.6; 1.6 and 2d.7; 1.6 and 2d.8; 1.6 and 2d.9; 1.6 and 2d.10; 1.6 and 2d.11; 1.6 and 2d.12; 1.7 and 2d.1; 1.7 and 2d.2; 1.7 and 2d.3; 1.7 and 2d.4; 1.7 and 2d.5; 1.7 and 2d.6; 1.7 and 2d.7; 1.7 and 2d.8; 1.7 and 2d.9; 1.7 and 2d.10; 1.7 and 2d.11; 1.7 and 2d.12; 1.12 and 2d.1; 1.12 and 2d.2; 1.12 and 2d.3; 1.12 and 2d.4; 1.12 and 2d.5; 1.12 and 2d.6; 1.12 and 2d.7; 1.12 and 2d.8; 1.12 and 2d.9; 1.12 and 2d.10; 1.12 and 2d.11; 1.12 and 2d.12; 1.14 and 2d.1; 1.14 and 2d.2; 1.14 and 2d.3; 1.14 and 2d.4; 1.14 and 2d.5; 1.14 and 2d.6; 1.14 and 2d.7; 1.14 and 2d.8; 1.14 and 2d.9; 1.14 and 2d.10; 1.14 and 2d.11; 1.14 and 2d.12; 1.15 and 2d.1; 1.15 and 2d.2; 1.15 and 2d.3; 1.15 and 2d.4; 1.15 and 2d.5; 1.15 and 2d.6; 1.15 and 2d.7; 1.15 and 2d.8; 1.15 and 2d.9; 1.15 and 2d.10; 1.15 and 2d.11 or 1.15 and 2d.12, in each case optionally in the form of the racemates, enantiomers or diastereomers thereof and optionally in the form of the pharmacologically acceptable acid addition salts, solvates and/or hydrates thereof.

Of the above-mentioned combinations, preferred ones according to the invention are those which contain as compound of formula 1 one of the compounds 1.1 1.5, 1.6, or 1.12, while those combinations which contain one of the compounds 1.1 or 1.12 are particularly important according to the invention. Of the above-mentioned combinations also preferred according to the invention are those which contain as compound 2d one of the compounds 2d.1, 2d.4, 2d.5, 2d.7, 2d.8, 2d.9, 2d.10, 2d.11 or 2d.12, while those combinations which contain one of the compounds 2d.1, 2d.4, 2d.5, 2d.7, 2d.8 or 2d.9 are particularly important according to the invention, and those combinations which contain one of the compounds 2d.1, 2d.4 or 2d.5 are of exceptional importance.

Other preferred medicament combinations according to the invention contain as an additional active substance, in addition to one or more, preferably one compound of formula 1, one or more, preferably one, EGFR-inhibitor 2e, optionally in combination with pharmaceutically acceptable excipients.

In such medicament combinations the EGFR-inhibitor 2e is selected for example from the group comprising 4-[(3-chloro-4-fluorophenyl)amino]-6-{[4-(morpholin-4-yl)-1-oxo-2-buten-1-yl]amino}-7-cyclopropylmethoxy-quinazoline, 4-[(3-chloro-4-fluorophenyl)amino]-6-{[4-(N,N-diethylamino)-1-oxo-2-buten-1-yl]amino}-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopentyloxy-quinazoline, 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline, 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline, 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N-cyclopropyl-N-methyl-amino)-1-oxo-2-buten-1-yl]amino}-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline, 4-[(3-ethynyl-phenyl)amino]-6,7-bis-(2-methoxy-ethoxy)-quinazoline, 4-[(3-chloro-4-fluorophenyl)amino]-7-[3-(morpholin-4-yl)-propyloxy]-6-[(vinylcarbonyl)amino]-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-{[3-chloro-4-(3-fluoro-benzyloxy)-phenyl]amino}-6-(5-{[(2-methanesulphonyl-ethyl)amino]methyl}-furan-2-yl)quinazoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-chloro-4-fluorophenyl)amino]-6-({4-[N,N-bis-(2-methoxy-ethyl)-amino]-1-oxo-2-buten-1-yl}aamino)-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]aamino}-quinazoline, 4-[(3-chloro-4-fluoro-phenyl)amino]-6-[2-(2,2-dimethyl-6-oxo-morpholin-4-yl)-ethoxy]-7-methoxy-quinazoline, 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline, 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-[1-(tert.-butyloxycarbonyl)-piperidin-4-yloxy]-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-(1-methyl-piperidin-4-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(methoxymethyl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-((S)-tetrahydrofuran-3-yloxy)-7-hydroxy-quinazoline, 4-[(3-chloro-4-fluoro-phenyl)amino]-6-(tetrahydropyran-4-yloxy)-7-(2-methoxy-ethoxy)-quinazoline, 4-[(3-chloro-4-fluoro-phenyl)amino]-6-{trans-4-[(dimethylamino)sulphonylamino]-cyclohexan-1-yloxy}-7-methoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{trans-4-[(morpholin-4-yl)sulphonylamino]-cyclohexan-1-yloxy}-7-methoxy-quinazoline, 4-[(3-chloro-4-fluoro-phenyl)amino]-6-(tetrahydropyran-4-yloxy)-7-(2-acetylamino-ethoxy)- quinazoline, 4-[(3-chloro-4-fluoro-phenyl)amino]-6-(tetrahydropyran-4-yloxy)-7-(2-methanesulphonylamino-ethoxy)-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(1-aminocarbonylmethyl-piperidin-4-yloxy)-7-methoxy-quinazoline, 4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline, 4-[(3-chloro-4-fluoro-phenyl)amino]-6-(trans-4-ethanesulphonylamino-cyclohexan- 1-yloxy)-7-methoxy-quinazoline, 4-[(3-chloro-4-fluoro-phenyl)amino]-6-(1-methanesulphonyl-piperidin-4-yloxy)-7-ethoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-chloro-4-fluoro-phenyl)amino]-6-(cis-4-acetylamino-cyclohexan-1-yloxy)-7-methoxy-quinazoline, 4-[(3-ethynyl-phenyl)amino]-6-[1-(tert.-butyloxycarbonyl)-piperidin-4-yloxy]-7-methoxy-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-chloro-4-fluoro-phenyl)amino]-6-{1-[(morpholin-4-yl)carbonyl]-piperidin-4-yloxy}-7-(2-methoxy-ethoxy)-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-chloro-4-fluoro-phenyl)amino]-6-(1-isopropyloxycarbonyl-piperidin-4-yloxy)-7-methoxy-quinazoline, 4-[(3-chloro-4-fluoro-phenyl)amino]-6-(cis-4-methylamino-cyclohexan-1-yloxy)-7-methoxy-quinazoline, 4-[(3-chloro-4-fluoro-phenyl)amino]-6-{cis-4-[N-(2-methoxy-acetyl)-N-methyl-amino]-cyclohexan-1-yloxy}-7-methoxy-quinazoline, 4-[(3-ethynyl-phenyl)amino]-6-(piperidin-4-yloxy)-7-methoxy-quinazoline, 4-[(3-ethynyl-phenyl)amino]-6-[1-(2-methoxy-acetyl)-piperidin-4-yloxy]-7-methoxy-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(cis-2,6-dimethyl-morpholin-4-yl)carbonyl]-piperidin-4-yloxy}-7-methoxy-quinazoline, 4-[(3-chloro-4-fluoro-phenyl)amino]-6-{1-[(2-methyl-morpholin-4-yl)carbonyl]-piperidin-4-yloxy}-7-methoxy-quinazoline, 4-[(3-chloro-4-fluoro-phenyl)amino]-6-{1-[(S,S)-(2-oxa-5-aza-bicyclo[2.2.1]hept-5-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-{1-[(2-methoxyethyl)carbonyl]-piperidin-4-yloxy}-7-methoxy-quinazoline, 4-[(3-chloro-4-fluoro-phenyl)amino]-6-{1-[(3-methoxypropyl-amino)-carbonyl]-piperidin-4-yloxy}-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)aamino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, cetuximab, trastuzumab, ABX-EGF and Mab ICR-62, optionally in the form of the racemates, enantiomers or diastereomers thereof, optionally in the form of the pharmacologically acceptable acid addition salts thereof, the solvates and/or hydrates thereof.

In such medicament combinations the EGFR-inhibitor 2e is preferably selected from among the 4-[(3-chloro-4-fluorophenyl)amino]-6-{[4-(morpholin-4-yl)-1-oxo-2-buten-1-yl]amino}-7-cyclopropylmethoxy-quinazoline, 4-[(3-chloro-4-fluorophenyl)amino]-6-{[4-(N,N-diethylamino)-1-oxo-2-buten-1-yl]amino}-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopentyloxy-quinazoline, 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline, 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline, 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N-cyclopropyl-N-methyl-amino)-1-oxo-2-buten-1-yl]amino}-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline, 4-[(3-ethynyl-phenyl)amino]-6,7-bis-(2-methoxy-ethoxy)-quinazoline, 4-[(3-chloro-4-fluorophenyl)amino]-7-[3-(morpholin-4-yl)-propyloxy]-6-[(vinylcarbonyl)amino]-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-{[3-chloro-4-(3-fluoro-benzyloxy)-phenyl]amino}-6-(5-{[(2-methanesulphonyl-ethyl)amino]methyl}-furan-2-yl)quinazoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-[2-(2,2-dimethyl-6-oxo-morpholin-4-yl)-ethoxy]-7-methoxy-quinazoline, 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline, 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-[1-(tert.-butyloxycarbonyl)-piperidin-4-yloxy]-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-(1-methyl-piperidin-4-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(methoxymethyl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-((S)-tetrahydrofuran-3-yloxy)-7-hydroxy-quinazoline, 4-[(3-chloro-4-fluoro-phenyl)aamino]-6-(tetrahydropyran-4-yloxy)-7-(2-methoxy-ethoxy)-quinazoline, 4-[(3-chloro-4-fluoro-phenyl)amino]-6-{trans-4-[(dimethylaamino)sulphonylamino]-cyclohexan-1-yloxy}-7-methoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{trans-4-[(morpholin-4-yl)sulphonylamino]-cyclohexan-1-yloxy}-7-methoxy-quinazoline, 4-[(3-chloro-4-fluoro-phenyl)amino]-6-(tetrahydropyran-4-yloxy)-7-(2-acetylamino-ethoxy)-quinazoline, 4-[(3-chloro-4-fluoro-phenyl)amino]-6-(tetrahydropyran-4-yloxy)-7-(2-methanesulphonylamino-ethoxy)-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(1-aminocarbonylmethyl-piperidin-4-yloxy)-7-methoxy-quinazoline, 4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-ethoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-chloro-4-fluoro-phenyl)amino]-6-(cis-4-acetylamino-cyclohexan-1-yloxy)-7-methoxy-quinazoline, 4-[(3-ethynyl-phenyl)amino]-6-[1-(tert.-butyloxycarbonyl)-piperidin-4-yloxy]-7-methoxy-quinazoline, 4-[(3-ethynyl-phenyl) amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-chloro-4-fluoro-phenyl)amino]-6-{1-[(morpholin-4-yl)carbonyl]-piperidin-4-yloxy}-7-(2-methoxy-ethoxy)-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-chloro-4-fluoro-phenyl)amino]-6-(1-isopropyloxycarbonyl-piperidin-4-yloxy)-7-methoxy-quinazoline, 4-[(3-chloro-4-fluoro-phenyl)amino]-6-(cis-4-methylamino-cyclohexan-1-yloxy)-7-methoxy-quinazoline, 4-[(3-chloro-4-fluoro-phenyl)amino]-6-{cis-4-[N-(2-methoxy-acetyl)-N-methyl-amino]-cyclohexan-1-yloxy}-7-methoxy-quinazoline, 4-[(3-ethynyl-phenyl)amino]-6-(piperidin-4-yloxy)-7-methoxy-quinazoline, 4-[(3-ethynyl-phenyl)amino]-6-[1-(2-methoxy-acetyl)-piperidin-4-yloxy]-7-methoxy-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(cis-2,6-dimethyl-morpholin-4-yl)carbonyl]-piperidin-4-yloxy}-7-methoxy-quinazoline, 4-[(3-chloro-4-fluoro-phenyl)amino]-6-{1-[(2-methyl-morpholin-4-yl)carbonyl]-piperidin-4-yloxy}-7-methoxy-quinazoline, 4-[(3-chloro-4-fluoro-phenyl)amino]-6-{1-[(S,S)-(2-oxa-5-aza-bicyclo[2.2.1]hept-5-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-{1-[(2-methoxyethyl)carbonyl]-piperidin-4-yloxy}-7-methoxy-quinazoline, 4-[(3-chloro-4-fluoro-phenyl)amino]-6-{1-[(3-methoxypropyl-amino)-carbonyl]-piperidin-4-yloxy}-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, and cetuximab, optionally in the form of the racemates, enantiomers or diastereomers thereof, optionally in the form of the pharmacologically acceptable acid addition salts thereof, the solvates and/or hydrates thereof.

Particularly preferably, the EGFR-inhibitors 2a used within the scope of the medicament combinations according to the invention are selected from the group comprising 4-[(3-chloro-4-fluorophenyl)amino]-6-{[4-(morpholin-4-yl)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]aamino}-7-[(S)-(tetrahydrofuran-3-yl)oxy]-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-ethynyl-phenyl)amino]-6,7-bis-(2-methoxy-ethoxy)-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{11-[2-(2-oxopyrrolidin-1-yl)ethyl]-piperidin-4-yloxy}-7-methoxy-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, and 4-[(3-chloro-4-fluoro-phenyl)amino]-6-{1-[(2-methoxyethyl)carbonyl]-piperidin-4-yloxy}-7-methoxy-quinazoline, optionally in the form of the racemates, enantiomers or diastereomers thereof, optionally in the form of the pharmacologically acceptable acid addition salts thereof, the solvates and/or hydrates thereof.

Particularly preferred medicament combinations according to the invention contain as EGFR-inhibitors 2e those compounds which are selected from the group comprising
4-[(3-chloro-4-fluorophenyl)amino]-6-{[4-(morpholin-4-yl)-1-oxo-2-buten-1-yl]-amino}-7-cyclopropylmethoxy-quinazoline (2e.1),
4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline (2e.2),
4-[(3-chloro-4-fluoro-phenyl)amino]-6-[2-((S)-6-methyl-2-oxo-morpholin-4-yl)-ethoxy]-7-methoxy-quinazoline (2e.3),
4-[(3-chloro-4-fluorophenyl)amino]-6-({4-[N-(2-methoxy-ethyl)-N-methyl-amino]-1-oxo-2-buten-1-yl}aamino)-7-cyclopropylmethoxy-quinazoline (2e.4),
4-[(3-ethynyl-phenyl)amino]-6,7-bis-(2-methoxy-ethoxy)-quinazoline (2e.5),
4-[(3-chloro-4-fluorophenyl)amino]-6-{[4-(morpholin-4-yl)-1-oxo-2-buten-1-yl]-amino}-7-[(tetrahydrofuran-2-yl)methoxy]-quinazoline (2e.6),
4-[(3-ethynyl-phenyl)amino]-6-{[4-(5,5-dimethyl-2-oxo-morpholin-4-yl)-1-oxo-2-buten-1-yl]amino}-quinazoline (2e.7),
4-[(3-chloro-4-fluoro-phenyl)amino]-6-(trans-4-methanesulphonylamino-cyclohexan-1-yloxy)-7-methoxy-quinazoline (2e.8),
4-[(3-chloro-4-fluoro-phenyl)amino]-6-(tetrahydropyran-3-yloxy)-7-methoxy-quinazoline (2e.9),
4-[(3-chloro-4-fluoro-phenyl)amino]-6-{1-[(morpholin-4-yl)carbonyl]-piperidin-4-yl-oxy}-7-methoxy-quinazoline (2e.10),
4-[(3-chloro-4-fluoro-phenyl)amino]-6-{11-[2-(2-oxopyrrolidin-1-yl)ethyl]-piperidin-4-yloxy}-7-methoxy-quinazoline (2e.11),
4-[(3-ethynyl-phenyl)amino]-6-(1-acetyl-piperidin-4-yloxy)-7-methoxy-quinazoline (2e.12),
4-[(3-ethynyl-phenyl)amino]-6-(1-methyl-piperidin-4-yloxy)-7-methoxy-quinazoline (2e.13),
4-[(3-ethynyl-phenyl)amino]-6-(1-methanesulphonyl-piperidin-4-yloxy)-7-methoxy-quinazoline (2e.14),
4-[(3-ethynyl-phenyl)amino]-6-{1-[(morpholin-4-yl)carbonyl]-piperidin-4-yloxy}-7-methoxy-quinazoline (2e.15),
4-[(3-chloro-4-fluoro-phenyl)amino]-6-{1-[(2-methoxyethyl)carbonyl]-piperidin-4-yloxy}-7-methoxy-quinazoline (2e.16),
4-[(3-chloro-4-fluoro-phenyl)amino]-6-[cis-4-(N-methanesulphonyl-N-methyl-amino)-cyclohexan-1-yloxy]-7-methoxy-quinazoline (2e.17),
4-[(3-chloro-4-fluoro-phenyl)amino]-6-[cis-4-(N-acetyl-N-methyl-amino)-cyclohexan-1-yloxy]-7-methoxy-quinazoline (2e.18),
4-[(3-chloro-4-fluoro-phenyl)amino]-6-(trans-4-methylamino-cyclohexan-1-yloxy)-7-methoxy-quinazoline (2e.19),
4-[(3-chloro-4-fluoro-phenyl)amino]-6-[trans-4-(N-methanesulphonyl-N-methyl-amino)-cyclohexan-1-yloxy]-7-methoxy-quinazoline (2e.20),
4-[(3-chloro-4-fluoro-phenyl)amino]-6-(trans-4-dimethylamino-cyclohexan-1-yloxy)-7-methoxy-quinazoline (2e.21),
4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline (2e.22),
4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline (2e.23),
4-[(3-chloro-4-fluoro-phenyl)amino]-6-(1-methanesulphonyl-piperidin-4-yloxy)-7-methoxy-quinazoline (2e.24) and
4-[(3-chloro-4-fluoro-phenyl)amino]-6-(1-cyano-piperidin-4-yloxy)-7-methoxy-quinazoline (2e.25),
   optionally in the form of the racemates, enantiomers or diastereomers thereof, optionally in the form of the pharmacologically acceptable acid addition salts thereof, the solvates and/or hydrates thereof.

By the acid addition salts with pharmacologically acceptable acids which the compounds 2e may possibly be capable of forming are meant for example salts selected from the group comprising the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrooxalate, hydrosuccinate, hydrobenzoate and hydro-p-toluenesulphonate, preferably the hydrochloride, hydrobromide, hydrosulphate, hydrophosphate, hydrofumarate and hydromethanesulphonate.

Examples of novel preferred medicament combinations of preferred compounds of formula 1 with the above-mentioned EGFR-inhibitors 2e are combinations containing the compounds 1.1 and 2e.1; 1.1 and 2e.2; 1.1 and 2e.3; 1.1 and 2e.4; 1.1 and 2e.5; 1.1 and 2e.6; 1.1 and 2e.7; 1.1 and 2e.8; 1.1 and 2e.9; 1.1 and 2e.10; 1.1 and 2e.11; 1.1 and 2e.12; 1.1 and 2e.13; 1.1 and 2e.14; 1.1 and 2e.15; 1.1 and 2e.16; 1.1 and 2e.17; 1.1 and 2e.18; 1.1 and 2e.19; 1.1 and 2e.20; 1.1 and 2e.21; 1.1 and 2e.22; 1.1 and 2e.23; 1.1 and 2e.24; 1.1 and 2e.25; 1.2 and 2e.1; 1.2 and 2e.2; 1.2 and 2e.3; 1.2 and 2e.4; 1.2 and 2e.5; 1.2 and 2e.6; 1.2 and 2e.7; 1.2 and 2e.8; 1.2 and 2e.9; 1.2 and 2e.10; 1.2 and 2e.11; 1.2 and 2e.12; 1.2 and 2e.13; 1.2 and 2e.14; 1.2 and 2e.15; 1.2 and 2e.16; 1.2 and 2e.17; 1.2 and 2e.18; 1.2 and 2el.9; 1.2 and 2e.20; 1.2 and 2e.21; 1.2 and 2e.22; 1.2 and 2e.23; 1.2 and 2e.24; 1 and 2e.25; 1.3 and 2e.1; 1.3 and 2e.2; 1.3 and 2e.3; 1.3 and 2e.4; 1.3 and 2e.5; 1.3 and 2e.6; 1.3 and 2e.7; 1.3 and 2e.8; 1.3 and 2e.9; 1.3 and 2e.10; 1.3 and 2e.11; 1.3 and 2e.12; 1.3 and 2e.13; 1.3 and 2e.14; 1.3 and 2e.15; 1.3 and 2e.16; 1.3 and 2e.17; 1.3 and 2e.18; 1.3 and 2e.19; 1.3 and 2e.20; 1.3 and 2e.21; 1.3 and 2e.22; 1.3 and 2e.23; 1.3 and 2e.24; 1.3 and 2e.25; 1.4 and 2e.1; 1.4 and 2e.2; 1.4 and 2e.3; 1.4 and 2e.4; 1.4 and 2e.5; 1.4 and 2e.6; 1.4 and 2e.7; 1.4 and 2e.8; 1.4 and 2e.9; 1.4 and 2e.10; 1.4 and 2e.11; 1.4 and 2e.12; 1.4 and 2e.13; 1.4 and 2e.14; 1.4 and 2e.15; 1.4 and 2e.16; 1.4 and 2e.17; 1.4 and 2e.18; 1.4 and 2e.19; 1.4 and 2e.20; 1.4 and 2e.21; 1.4 and 2e.22; 1.4 and 2e.23; 1.4 and 2e.24; 1.4 and 2e.25; 1.5 and 2e.1; 1.5 and 2e.2; 1.5 and 2e.3; 1.5 and 2e.4; 1.5 and 2e.5; 1.5 and 2e.6;1.5 and 2e.7;1.5 and 2e.8; 1.5 and 2e.9; 1.5 and 2e.10; 1.5 and 2e.11; 1.5 and 2e.12; 1.5 and 2e.13; 1.5 and 2e.14; 1.5 and 2e.15; 1.5 and 2e.16; 1.5 and 2e.17; 1.5 and 2e.18; 1.5 and 2e.19; 1.5 and 2e.20; 1.5 and 2e.21; 1.5 and 2e.22; 1.5 and 2e.23; 1.5 and 2e.24; 1.5 and 2e.25; 1.6 and 2e.1; 1.6 and 2e.2; 1.6 and 2e.3; 1.6 and 2e.4; 1.6 and 2e.5; 1.6 and 2e.6;1.6 and 2e.7; 1.6 and 2e.8; 1.6 and 2e.9; 1.6 and 2e.10; 1.6 and 2e.11; 1.6 and 2e.12; 1.6 and 2e.13; 1.6 and 2e.14; 1.6 and 2e.15;1.6 and 2e.16; 1.6 and 2e.17; 1.6 and 2e.18; 1.6 and 2e.19; 1.6 and 2e.20; 1.6 and 2e.21; 1.6 and 2e.22; 1.6 and 2e.23; 1.6 and 2e.24; 1.6 and 2e.25; 1.7 and 2e.1; 1.7 and 2e.2; 1.7 and 2e.3; 1.7 and 2e.4; 1.7 and 2e.5; 1.7 and 2e.6;1.7 and 2e.7;1.7 and 2e.8; 1.7 and 2e.9; 1.7 and 2e.10; 1.7 and 2e.11; 1.7 and 2e.12; 1.7 and 2e.13; 1.7 and 2e.14; 1.7 and 2e.15; 1.7 and 2e.16; 1.7 and 2e.17; 1.7 and 2e.18; 1.7 and 2e.19; 1.7 and 2e.20; 1.7 and 2e.21;1.7 and 2e.22; 1.7 and 2e.23; 1.7 and 2e.24; 1.7 and 2e.25; 1.12 and 2e.1; 1.12 and 2e.2; 1.12 and 2e.3; 1.12 and 2e.4; 1.12 and 2e.5; 1.12 and 2e.6; 1.12 and 2e.7; 1.12 and 2e.8; 1.12 and 2e.9; 1.12 and 2e.10; 1.12 and 2e.11; 1.12 and 2e.12; 1.12 and 2e.13; 1.12 and 2e.14; 1.12 and 2e.15; 1.12 and 2e.16; 1.12 and 2e.17; 1.12 and 2e.18; 1.12 and 2e.19; 1.12 and 2e.20; 1.12 and 2e.21; 1.12 and 2e.22; 1.12 and 2e.23; 1.12 and 2e.24; 1.12 and 2e.25; 1.14 and 2e.1; 1.14 and 2e.2; 1.14 and 2e.3; 1.14 and 2e.4; 1.14 and 2e.5; 1.14 and 2e.6; 1.14 and 2e.7; 1.14 and 2e.8; 1.14 and 2e.9; 1.14 and 2e.10; 1.14 and 2e.11; 1.14 and 2e.12; 1.14 and 2e.13; 1.14 and 2e.14; 1.14 and 2e.15; 1.14 and 2e.16; 1.14 and 2e.17; 1.14 and 2e.18; 1.14 and 2e.19; 1.14 and 2e.20; 1.14 and 2e.21; 1.14 and 2e.22; 1.14 and 2e.23; 1.14 and 2e.24; 1.14 and 2e.25; 1.15 and 2e.1; 1.15 and 2e.2; 1.15 and 2e.3; 1.15 and 2e.4; 1.15 and 2e.5; 1.15 and 2e.6; 1.15 and 2e.7; 1.15 and 2e.8; 1.15 and 2e.9; 1.15 and 2e.10; 1.15 and 2e.11; 1.15 and 2e.12; 1.15 and 2e.13; 1.15 and 2e.14; 1.15 and 2e.15; 1.15 and 2e.16,1.15 and 2e.17; 1.15 and 2e.18; 1.15 and 2e.19; 1.15 and 2e20; 1.15 and 2e.21; 1.15 and 2e.22; 1.15 and 2e.23; 1.15 and 2e.24 or 1.15 and 2e.25, in each case optionally in the form of the racemates, enantiomers or diastereomers thereof and optionally in the form of the pharmacologically acceptable acid addition salts, solvates and/or hydrates thereof.

Of the above-mentioned combinations, preferred ones according to the invention are those which contain as compound of formula 1 one of the compounds 1.1, 1.5, 1.6, or 1.12, while those combinations which contain one of the compounds 1.1 or 1.12 are particularly important according to the invention. Of the above-mentioned combinations also preferred according to the invention are those which contain as the compound 2e one of the compounds 2e.1, 2e.2, 2e.3, 2e.4, 2e.10, 2e.11, 2e.14, 2e.16, 2e.17, 2e.18, 2e.19, 2e.20, 2e.21, 2e.22, 2e.23, 2e.24 or 2e.25 , while those combinations which contain one of the compounds 2e.2, 2e.3 or 2e.4 are particularly important according to the invention.

The novel medicament combinations comprising compounds of formula 1 with at least one other active substance 2 are not restricted to binary combinations of active substances. The combinations mentioned above, partly by way of example, which contain in addition to a compound of formula I one other active substance 2, may also contain a third or fourth, preferably a third active substance, which is also selected from the above-mentioned group of anticholinergics (2a), PDE-IV inhibitors (2b), steroids (2c), LTD4-antagonists (2d) and EGFR-inhibitors (2e).

Particularly preferred combinations which contain two other active substances in addition to a compound of formula 1 are selected from the active substance combinationslisted below. These are medicament combinations which may contain, for example:
A) a compound of formula 1, an anticholinergic (2a), a PDEIV inhibitor (2b);
B) a compound of formula 1, an anticholinergic (2a), a steroid (2c);
C) a compound of formula 1, an anticholinergic (2a), an LTD4 antagonist (2d);
D) a compound of formula 1, an anticholinergic (2a), an EGFR inhibitor (2e);
E) a compound of formula 1, a PDEIV inhibitor (2b), a steroid (2c);
F) a compound of formula 1, a PDEIV inhibitor (2b), an LTD4 antagonist (2d);
G) a compound of formula 1, a PDEIV inhibitor (2b), an EGFR inhibitor (2e);
H) a compound of formula 1, a steroid (2c), an LTD4 antagonist (2d);
I) a compound of formula 1, a steroid (2c), an EGFR inhibitor (2e);
J) a compound of formula 1, an LTD4 antagonist (2d), an EGFR inhibitor (2e).

Particularly preferred examples of medicament combinations of the above-mentioned group A are selected from the group comprising the following combinations:
compounds 1.1 and 2a.1 and 2b.2; 1.1 and 2a.1 and 2b.4; 1.1 and 2a.1 and 2b.11; 1.1 and 2a.1 and 2b.19; 1.1 and 2a.9.1 and 2b.2; 1.1 and 2a.9.1 and 2b.4; 1.1 and 2a.9.1 and 2b.11; 1.1 and 2a.9.1 and 2b.19; 1.1 and 2a.9.2 and 2b.2; 1.1 and 2a.9.2 and 2b.4; 1.1 and 2a.9.2 and 2b.11; 1.1 and 2a.9.2 and 2b.19; 1.1 and 2a.10.1 and 2b.2; 1.1 and 2a.10.1 and 2b.4; 1.1 and 2a.10.1 and 2b.11; 1.1 and 2a.10.1 and 2b.19; 1.1 and 2a.10.2 and 2b.2; 1.1 and 2a.10.2 and 2b.4; 1.1 and 2a.10.2 and 2b.11; 1.1 and 2a.10.2 and 2b.19; 1.1 and 2a.11.1 and 2b.2; 1.1 and 2a.11.1 and 2b.4; 1.1 and 2a.11.1 and 2b.11; 1.1 and 2a.11.1 and 2b.19; 1.1 and 2a.11.6 and 2b.2; 1.1 and 2a.11.6 and 2b.4; 1.1 and 2a.11.6 and 2b.11; 1.1 and 2a.11.6 and 2b.19; 1.12 and 2a.1 and 2b.2; 1.12 and 2a.1 and 2b.4; 1.12 and 2a.1 and 2b.11; 1.12 and 2a.1 and 2b.19; 1.12 and 2a.9.1 and 2b.2; 1.12 and 2a.9.1 and 2b.4; 1.12 and 2a.9.1 and 2b.11; 1.12 and 2a.9.1 and 2b.19; 1.12 and 2a.9.2 and 2b.2; 1.12 and 2a.9.2 and 2b.4; 1.12 and 2a.9.2 and 2b.11; 1.12 and 2a.9.2 and 2b.19; 1.12 and 2a.10.1 and 2b.2; 1.12 and 2a.10.1 and 2b.;4 1.12 and 2a.10.1 and 2b.11; 1.12 and 2a.10.1 and 2b.19; 1.12 and 2a.10.2 and 2b.2; 1.12 and 2a.10.2 and 2b.4; 1.12 and 2a.10.2 and 2b.11; 1.12 and 2a.10.2 and 2b.19; 1.12 and 2a.11.12 and 2b.2; 1.12 and 2a.11.12 and 2b.4; 1.12 and 2a.11.12 and 2b.11; 1.12 and 2a.11.12 and 2b.19; 1.12 and 2a.11.6 and 2b.2; 1.12 and 2a.11.6 and 2b.4; 1.12 and 2a.11.6 and 2b.11; 1.12 and 2a.11.6 and 2b.19, in each case optionally in the form of the racemates, enantiomers or diastereomers thereof and optionally in the form of the pharmacologically acceptable acid addition salts, solvates and/or hydrates thereof.

Particularly preferred examples of particularly preferred medicament combinations of the above-mentioned group B according to the invention are selected from the group comprising the following combinations:
compounds 1.1 and 2a.1 and 2c.8; 1.1 and 2a.1 and 2c.9; 1.1 and 2a.1 and 2c.10; 1.1 and 2a.1 and 2c.11; 1.1 and 2a.1 and 2c.17; 1.1 and 2a.9.1 and 2c.8; 1.1 and 2a.9.1 and 2c.9; 1.1 and 2a.9.1 and 2e.10; 1.1 and 2a.9.1 and 2c.11; 1.1 and 2a.9.1 and 2c.17; 1.1 and 2a.9.2 and 2c.8; 1.1 and 2a.9.2 and 2c.9; 1.1 and 2a.9.2 and 2e.10; 1.1 and 2a.9.2 and 2c.11; 1.1 and 2a.9.2 and 2c.17; 1.1 and 2a.10.1 and 2c.8; 1.1 and 2a.10.1 and 2c.9; 1.1 and 2a.10.1 and 2c.10; 1.1 and 2a.10.1 and 2c.11; 1.1 and 2a.10.1 and 2c.17; 1.1 and 2a.10.2 and 2c.8; 1.1 and 2a.10.2 and 2c.9; 1.1 and 2a.10.2 and 2c.10; 1.1 and 2a.10.2 and 2c.11; 1.1 and 2a.10.2 and 2e.17; 1.1 and 2a.11.1 and 2c.8; 1.1 and 2a.11.1 and 2c.9; 1.1 and 2a.11.1 and 2c.10; 1.1 and 2a.11.1 and 2c.11; 1.1 and 2a.11.1 and 2c.17; 1.1 and 2a.11.6 and 2c.8; 1.1 and 2a.11.6 and 2c.9; 1.1 and 2a.11.6 and 2c.10; 1.1 and 2a.11.6 and 2c.11; 1.1 and 2a.11.6 and.2c.17; 1.12 and 2a.1 and 2c.8; 1.12 and 2a.1 and 2c.9; 1.12 and 2a.1 and 2e.10; 1.12 and 2a.1 and 2e.11; 1.12 and 2a.1 and 2c.17; 1.12 and 2a.9.1 and 2c.8; 1.12 and 2a.9.1 and 2c.9; 1.12 and 2a.9.1 and 2c.10; 1.12 and 2a.9.1 and 2c.11; 1.12 and 2a.9.1 and 2c.17; 1.12 and 2a.9.2 and 2c.8; 1.12 and 2a.9.2 and 2c.9; 1.12 and 2a.9.2 and 2c.10; 1.12 and 2a.9.2 and 2c.11; 1.12 and 2a.9.2 and 2c.17; 1.12 and 2a.10.1 and 2c.8; 1.12 and 2a.10.1 and 2c.9; 1.12 and 2a.10.1 and 2c.10; 1.12 and 2a.10.1 and 2c.11; 1.12 and 2a.10.1 and 2c.17; 1.12 and 2a.10.2 and 2c.8; 1.12 and 2a.10.2 and 2c.9; 1.12 and 2a.10.2 and 2c.10; 1.12 and 2a.10.2 and 2c.11; 1.12 and 2a.10.2 and 2c.17; 1.12 and 2a.11.12 and 2c.8; 1.12 and 2a.11.12 and 2c.9; 1.12 and 2a.11.12 and 2c.10; 1.12 and 2a.11.12 and 2c.11; 1.12 and 2a.11.12 and 2c.17; 1.12 and 2a.11.6 and 2c.8; 1.12 and 2a.11.6 and 2c.9; 1.12 and 2a.11.6 and 2c.10; 1.12 and 2a.11.6 and 2c.11: 1.12 and 2a.11.6 and 2c.17,, in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group C are selected from the group comprising the following combinations:
compounds 1.1 and 2a.1 and 2d.1; 1.1 and 2a.1 and 2d.4; 1.1 and 2a.1 and 2d.5; 1.1 and 2a.1 and 2d.8; 1.1 and 2a.9.1 and 2d.1; 1.1 and 2a.9.1 and 2d.4; 1.1 and 2a.9.1 and 2d.5; 1.1 and 2a.9.1 and 2d.8; 1.1 and 2a.9.2 and 2d.1; 1.1 and 2a.9.2 and 2d.4; 1.1 and 2a.9.2 and 2d.5; 1.1 and 2a.9.2 and 2d.8; 1.1 and 2a.10.1 and 2d.1; 1.1 and 2a.10.1 and 2d.4; 1.1 and 2a.10.1 and 2d.5; 1.1 and 2a.10.1 and 2d.8; 1.1 and 2a.10.2 and 2d.1; 1.1 and 2a.10.2 and 2d.4; 1.1 and 2a.10.2 and 2d.5; 1.1 and 2a.10.2 and 2d.8; 1.1 and 2a.11.1 and 2d.1; 1.1 and 2a.11.1 and 2d.4; 1.1 and 2a.11.1 and 2d.5; 1.1 and 2a.11.1 and 2d.8; 1.1 and 2a.11.6 and 2d.1; 1.1 and 2a.11.6 and 2d.4; 1.1 and 2a.11.6 and 2d.5; 1.1 and 2a.11.6 and 2d.8; 1.12 and 2a.1 and 2d.1; 1.12 and 2a.1 and 2d.4; 1.12 and 2a.1 and 2d.5; 1.12 and 2a.1 and 2d.8; 1.12 and 2a.9.1 and 2d.1; 1.12 and 2a.9.1 and 2d.4; 1.12 and 2a.9.1 and 2d.5; 1.12 and 2a.9.1 and 2d.8; 1.12 and 2a.9.2 and 2d.1; 1.12 and 2a.9.2 and 2d.4; 1.12 and 2a.9.2 and 2d.5; 1.12 and 2a.9.2 and 2d.8; 1.12 and 2a.10.1 and 2d.1; 1.12 and 2a.10.1 and 2d.4; 1.12 and 2a.10.1 and 2d.5; 1.12 and 2a.10.1 and 2d.8; 1.12 and 2a.10.2 and 2d.1; 1.12 and 2a.10.2 and 2d.4; 1.12 and 2a.10.2 and 2d.5; 1.12 and 2a.10.2 and 2d.8; 1.12 and 2a.11.12 and 2d.1; 1.12 and 2a.11.12 and 2d.4; 1.12 and 2a.11.12 and 2d.5; 1.12 and 2a.11.12 and 2d.8; 1.12 and 2a.11.6 and 2d.1; 1.12 and 2a.11.6 and 2d.4; 1.12 and 2a.11.6 and 2d.5; 1.12 and 2a.11.6 and 2d.8, in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group D are selected from the group comprising the following combinations: compounds 1.1 and 2a.1 and 2e.2; 1.1 and 2a.1 and 2e.3; 1.1 and 2a.1 and 2e.4; 1.1 and 2a.1 and 2e.10; 1.1 and 2a.9.1 and 2e.2; 1.1 and 2a.9.1 and 2e.3; 1.1 and 2a.9.1 and 2e.4; 1.1 and 2a.9.1 and 2e.10; 1.1 and 2a.9.2 and 2e.2; 1.1 and 2a.9.2 and 2e.3; 1.1 and 2a.9.2 and 2e.4; 1.1 and 2a.9.2 and 2e.10; 1.1 and 2a.10.1 and 2e.2; 1.1 and 2a.10.1 and 2e.3; 1.1 and 2a.10.1 and 2e.4; 1.1 and 2a.10.1 and 2e.10; 1.1 and 2a.10.2 and 2e.2; 1.1 and 2a.10.2 and 2e.3; 1.1 and 2a.10.2 and 2e.4; 1.1 and 2a.10.2 and 2e.10; 1.1 and 2a.11.1 and 2e.2; 1.1 and 2a.11.1 and 2e.3; 1.1 and 2a.11.1 and 2e.4; 1.1 and 2a.11.1 and 2e.10; 1.1 and 2a.11.6 and 2e.2; 1.1 and 2a.11.6 and 2e.3; 1.1 and 2a.11.6 and 2e.4; 1.1 and 2a.11.6 and 2e.10; 1.12 and 2a.1 and 2e.2; 1.12 and 2a.1 and 2e.3; 1.12 and 2a.1 and 2e.4; 1.12 and 2a.1 and 2e.10; 1.12 and 2a.9.1 and 2e.2; 1.12 and 2a.9.1 and 2e.3; 1.12 and 2a.9.1 and 2e.4; 1.12 and 2a.9.1 and 2e.10; 1.12 and 2a.9.2 and 2e.2; 1.12 and 2a.9.2 and 2e.3; 1.12 and 2a.9.2 and 2e.4; 1.12 and 2a.9.2 and 2e.10; 1.12 and 2a.10.1 and 2e.2; 1.12 and 2a.10.1 and 2e.3; 1.12 and 2a.10.1 and 2e.4; 1.12 and 2a.10.1 and 2e.10; 1.12 and 2a.10.2 and 2e.2; 1.12 and 2a.10.2 and 2e.3; 1.12 and 2a.10.2 and 2e.4; 1.12 and 2a.10.2 and 2e.10; 1.12 and 2a.11.12 and 2e.2; 1.12 and 2a.11.12 and 2e.3; 1.12 and 2a.11.12 and 2e.4; 1.12 and 2a.11.12 and 2e.10; 1.12 and 2a.11.6 and 2e.2; 1.12 and 2a.11.6 and 2e.3; 1.12 and 2a.11.6 and 2e.4; 1.12 and 2a.11.6 and 2e.10, in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group E are selected from the group comprising the following combinations:
compounds 1.1 and 2c.8 and 2b.2; 1.1 and 2c.8 and 2b.4; 1.1 and 2c.8 and 2b.11; 1.1 and 2c.8 and 2b.19; 1.1 and 2c.9 and 2b.2; 1.1 and 2c.9 and 2b. 1.1 and 2c.9 and 2b.11; 1.1 and 2c.9 and 2b.19; 1.1 and 2c.10 and 2b.2; 1.1 and 2c.10 and 2b.4; 1.1 and 2c.10 and 21; 1.1 and 2c.10 2b.19; Li and 2c.11 9 2b.2; and 2c.11 and 2b.4; 1.1 and 2c.11 a .1 and 2c.17 and 22; 1.1 L and 2c . 2 c.17 and 2b.4; 1.1 and 2c.17 and 2b.11; 1.1 and 2c.17 and 2b..9 1.1 and 2c.8 and 2bc.8 and 2b.4;1.12 and 2c.8 and 2b.11; 1.12 and 2c.8 and 2b.19; 1.12 and 2c.9 and 2b.2; 1.12 and 2c.9 and 2b.4; 1.12 and 2c.9 and 2b.11; 1.12 and 2c.9 and 2b.19; 1.12 and 2c.10 and 2b.2; 1.12 and 2c.10 and 2b.4; 1.12 and 2c.10 and 2b.11; 1.12 and 2c.10 and 2b.19; 1.12 and 2c.11 and 2b.2; 1.12 and 2c.11 and 2b.4; 1.12 and 2c.11 and 2b.11; 1.12 and 2c.11 and 2b.19,1.12 and 2c.17 and 2b.2; 1.12 and 2c.17 and 2b.4; 1.12 and 2c.17 and 2b.11; 1.12 and 2c.17 and 2b.19; in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group F are selected from the group comprising the following combinations:
compounds 1.1 and 2d.1 and 2b.2; 1.1 and 2d.1 and 2b.4; 1.1 and 2d.1 and 2b.11; 1.1 and 2d.1 and 2b.19; 1.1 and 2d.4 and 2b.2; 1.1 and 2d.4 and 2b.4; 1.1 and 2d.4 and 2b.11; 1.1 and 2d.4 and 2b.19; 1.1 and 2d.5 and 2b.2; 1.1 and 2d.5 and 2b.4; 1.1 and 2d.5 and 2b.11; 1.1 and 2d.5 and 2b.19; 1.1 and 2d.8 and 2b.2; 1.1 and 2d.8 and 2b.4; 1.1 and 2d.8 and 2b.11; 1.1 and 2d.8 and 2b.19; 1.12 and 2d.1 and 2b.2; 1.12 and 2d.1 and 2b.4; 1.12 and 2d.1 and 2b.11; 1.12 and 2d.1 and 2b.19; 1.12 and 2d.4 and 2b.2; 1.12 and 2d.4 and 2b.4; 1.12 and 2d.4 and 2b.11; 1.12 and 2d.4 and 2b.19; 1.12 and 2d.5 and 2b.2; 1.12 and 2d.5 and 2b.4; 1.12 and 2d.5 and 2b.11; 1.12 and 2d.5 and 2b.19; 1.12 and 2d.8 and,2b.2; 1.12 and 2d.8 and 2b.4; 1.12 and 2d.8 and 2b.11; 1.12 and 2d.8 and 2b.19, in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group G are selected from the group comprising the following combinations:
compounds 1.1 and 2e.2 and 2b.2; 1.1 and 2e.2 and 2b.4; 1.1 and 2e.2 and 2b.11; 1.1 and 2e.2 and 2b.19; 1.1 and 2e.3 and 2b.2; 1.1 and 2e.3 and 2b.4; 1.1 and 2e.3 and 2b.11; 1.1 and 2e.3 and 2b.19; 1.1 and 2e.4 and 2b.2; 1.1 and 2e.4 and 2b.4; 1.1 and 2e.4 and 2b.11; 1.1 and 2e.4 and 2b.19; 1.1 and 2e.10 and 2b.2; 1.1 and 2e.10 and 2b.4; 1.1 and 2e.10 and 2b.11; 1.1 and 2e.10 and 2b.19; 1.12 and 2e.2 and 2b.2; 1.12 and 2e.2 and 2b.4; 1.12 and 2e.2 and 211;12and e.2 and 2b.19; 1.12 and 2e.3 and 2b.2; 1.12 and 2e.3 and 2b.4; 1.12 and 2e.3 and 2b.11; 1.12 and 2e.3 and 2b.19; 1.12 and 2e.4 and 2b.2; 1.12 and 2e.4 and 2b.4; 1.12 and 2e.4 and 2b.11; 1.12 and 2e.4 and 2b.19; 1.12 and 2e.10 and 2b.2; 1.12 and 2e.10 and 2b.4; 1.12 and 2e.10 and 2b.11; 1.12 and 2e.10 and 2b.19, in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group H are selected from the group comprising the following combinations:
compounds 1.1 and 2c.8 and 2d.1; 1.1 and 2c.8 and 2d.4; 1.1 and 2c.8 and 2d.5; 1.1 and 2c.8 and 2d.8; 1.1 and 2c.9 and 2d.1; 1.1 and 2c.9 and 2d.4; 1.1 and 2c.9 and 2d.5; 1.1 and 2c.9 and 2d.8; 1.1 and 2c.10 and 2d.1; 1.1 and 2c.10 and 2d.4; 1.1 and 2c.10 and 2d.5; 1.1 and 2c.10 and2d.8; 1.1 and 2c.11 and2d.1; 1.1 and 2c.11 and2d.4; 1.1 and2c.11 and 2d.5; 1.1 and 2c.11 and 2d.8; 1.1 and 2c.17 and 2d.1; 1.1 and 2c.17 and 2d.4; 1.1 and 2c.17.and 2d.5; 1.1 and 2c.17 and 2d.8; 1.12 and 2c.8 and 2d.1; 1.12 and 2c.8 and 2d.4; 1.12 and 2c.8 and 2d.5; 1.12 and 2c.8 and 2d.8; 1.12 and 2c.9 and 2d.1; 1.12 and 2c.9 and 2d.4; 1.12 and 2c.9 and 2d.5; 1.12 and 2c.9 and 2d.8; 1.12 and 2c.10 and 2d.1; 1.12 and 2c.10 and 2d.4; 1.12 and 2c.10 and 2d.5; 1.12 and 2c.10 and 2d.8; 1.12 and 2c.11 and 2d.1; 1.12 and 2c.11 and 2d.4; 1.12 and 2c.11 and 2d.5;,1.12 and 2c.11 and 2d.8,1.12 and 2c.17 and 2d.1; 1.12 and 2c.17 and 2d.4; 1.12 and 2c.17 and 2d.5; 1.12 and 2c.17 and 2d.8; in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group I are selected from the group comprising the following combinations:
compounds 1.1 and 2c.8 and 2e.2; 1.1 and 2c.8 and 2e.3; 1.1 and 2c.8 and 2e.4; 1.1 and 2c.8 and 2e.10; 1.1 and 2c.9 and 2e.2; 1.1 and 2c.9 and 2e.3; 1.1 and 2c.9 and 2e.4; 1.1 and 2c.9 and 2e.10; 1.1 and 2c.10 and 2e.2; 1.1 and 2c.10 and 2e.3; 1.1 and 2c.10 and 2e.4; 1.1 and 2c.10 and 2e.10; 1.1 and 2c.11 and 2e.2; 1.1 and 2c.11 and 2e.3; 1.1 and 2c.11 and 2e.4; 1.1 and 2c.11 and 2e.10; 1.1 and 2c.17 and 2e.2; 1.1 and 2c.17 and 2e.3; 1.1 and 2c.17.and 2e.4; 1.1 and 2c.17 and 2e.10; 1.12 and 2c.8 and 2e.2; 1.12 and 2c.8 and 2e.3; 1.12 and 2c.8 and 2e.4; 1.12 and 2c.8 and 2e.10; 1.12 and 2c.9 and 2e.2; 1.12 and 2c.9 and 2e.3; 1.12 and 2c.9 and 2e.4; 1.12 and 2c.9 and 2e.10; 1.12 and 2c.10 and 2e.2; 1.12 and 2c.10 and 2e.3; 1.12 and 2c.10 and 2e.4; 1.12 and 2c.10 and 2e.10; 1.12 and 2c.11 and 2e.2; 1.12 and 2c.11 and 2e.3; 1.12 and 2c.11 and 2e.4; 1.12 and 2c.11 and 2e.10, 1.12 and 2c.17 and 2e.2; 1.12 and 2c.17 and 2e.3; 1.12 and 2c.17 and 2e.4; 1.12 and 2c.17 and 2e.10; in each case optionally in the form of the racemates, enantiomers or diastereomers thereof and optionally in the form of the pharmacologically acceptable acid addition salts, solvates and/or hydrates thereof.

Particularly preferred examples of medicament combinations of the above-mentioned group J are selected from the group comprising the following combinations:
compounds 1.1 and 2d.1 and 2e.2; 1.1 and 2d.1 and 2e.3; 1.1 and 2d.1 and 2e.4; 1.1 and 2d.1 and 2e.10; 1.1 and 2d.4 and 2e.2; 1.1 and 2d.4 and 2e.3; 1.1 and 2d.4 and 2e.4; 1.1 and 2d.4 and 2e.10; 1.1 and 2d.5 and 2e.2; 1.1 and 2d.5 and 2e.3; 1.1 and 2d.5 and 2e.4; 1.1 and 2d.5 and 2e.10; 1.1 and 2d.8 and 2e.2; 1.1 and 2d.8 and 2e.3; 1.1 and 2d.8 and 2e.4; 1.1 and 2d.8 and 2e.10; 1.12 and 2d.1 and 2e.2; 1.12 and 2d.1 and 2e.3; 1.12 and 2d.1 and 2e.4; 1.12 and 2d.1 and 2e.10; 1.12 and 2d.4 and 2e.2; 1.12 and 2d.4 and 2e.3; 1.12 and 2d.4 and 2e.4;.1.12 and 2d.4 and 2e.10; 1.12 and 2d.5 and 2e.2; 1.12 and 2d.5 and 2e.3; 1.12 and 2d.5 and 2e.4; 1.12 and 2d.5 and 2e.10; 1.12 and 2d.8 and 2e.2; 1.12 and 2d.8 and 2e.3; 1.12 and 2d.8 and 2e.4; 1.12 and 2d.8 and 2e.10, in each case optionally in the form of the racemates, enantiomers or diastereomers thereof and optionally in the form of the pharmacologically acceptable acid addition salts, solvates and/or hydrates thereof.

Of outstanding importance according to the invention are all those medicament combinations disclosed within the scope of the present invention which contain the compounds of formula 1 in the form of the R-enantiomers thereof.

Unless otherwise stated, the alkyl groups are straight-chained or branched alkyl groups having 1 to 4 carbon atoms. The following are mentioned by way of example: methyl, ethyl, propyl or butyl. In some cases the abbreviations Me, Et, Prop or Bu are used to denote the groups methyl, ethyl, propyl or butyl. Unless otherwise stated, the definitions propyl and butyl include all the possible isomeric forms of the groups in question. Thus, for example, propyl includes n-propyl and iso-propyl, butyl includes iso-butyl, sec.butyl and tert.-butyl, etc.

Unless otherwise stated, the cycloalkyl groups are alicyclic groups with 3 to 6 carbon atoms. They are the cyclopropyl, cyclobutyl, cyclopentyl and cyclohexyl groups. Cyclopropyl is particularly important within the scope of the present invention.

Unless otherwise stated, the alkylene groups are branched and unbranched double-bonded alkyl bridges with 1 to 4 carbon atoms . Examples include: methylene, ethylene, propylene or butylene.

Unless otherwise stated, the alkylene-halogen groups are branched and unbranched double-bonded alkyl bridges with 1 to 4 carbon atoms which are mono-, di- or trisubstituted, preferably disubstituted, by a halogen. Accordingly, unless otherwise stated, alkylene-OH-groups are branched and unbranched double-bonded alkyl bridges with 1 to 4 carbon atoms which are mono-, di- or trisubstituted, preferably monosubstituted, by a hydroxy.

Unless otherwise stated, the term alkyloxy groups denotes branched and unbranched alkyl groups with 1 to 4 carbon atoms which are linked via an oxygen atom. Examples include: methyloxy, ethyloxy, propyloxy or butyloxy. In some cases the abbreviations MeO, EtO, PropO or BuO may be used to denote the methyloxy, ethyloxy, propyloxy or butyloxy groups. Unless otherwise stated, the definitions propyloxy and butyloxy include all the possible isomeric forms of the groups in question. Thus, for example, propyloxy includes n-propyloxy and iso-propyloxy, butyloxy includes iso-butyloxy, sec.butyloxy and tert.-butyloxy, etc. In some cases the term alkoxy may be used instead of alkyloxy within the scope of the present invention. The groups methyloxy, ethyloxy, propyloxy or butyloxy may therefore also be referred to by the names methoxy, ethoxy, propoxy or butoxy.

Unless otherwise stated, the term alkylene-alkyloxy refers to branched and unbranched double-bonded alkyl bridges with 1 to 4 carbon atoms which are mono-, di- or trisubstituted, preferably monosubstituted, by an alkyloxy group.

Unless otherwise stated, the term —O—CO-alkyl groups refers to branched and unbranched alkyl groups with 1 to 4 carbon atoms which are linked by an ester group. The alkyl groups are attached directly to the carbonyl carbon of the ester group. The term —O—CO-alkyl-halogen should be understood analogously. The group —O—CO—CF₃ denotes trifluoroacetate.

Halogen within the scope of the present invention denotes fluorine, chlorine, bromine or iodine. Unless stated otherwise, fluorine and bromine are the preferred halogens. The group CO denotes a carbonyl group.

Within the scope of the present invention by a pharmaceutical combination of components 1 and 2 is meant the joint administration of both active substances in a single preparation or formulation or the separate administration of the two active substances in separate formulations. If the active substances 1 and 2 are administered in separate formulations, this separate administration may be done simultaneously or at different times, i.e. successively.

In one aspect the present invention relates to the above-mentioned medicament combinations which contain in addition to therapeutically effective amounts of 1 and 2 a pharmaceutically acceptable carrier. In one aspect the present invention relates to the above-mentioned pharmaceutical compositions which do not contain contain a pharmaceutically acceptable carrier in addition to therapeutically effective amounts of 1 and 2.

The present invention also relates to the use of therapeutically effective amounts of the active substances 1 for preparing a pharmaceutical composition also containing one or more, preferably one active substance 2 for the treatment of inflammatory and obstructive respiratory complaints, for inhibiting premature labour in midwifery (tocolysis), for restoring sinus rhythm in the heart in atrioventricular block, for correcting bradycardic heart rhythm disorders (antiarrhythmic), for treating circulatory shock (vasodilatation and increasing the heart volume) as well as for the treatment of skin irritations and inflammation.

In a preferred aspect the present invention relates to the use of therapeutically effective amounts of the active substance 1 for preparing a pharmaceutical composition also containing one or more, preferably one, active substance 2 for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably the medicament combinations according to the invention are used as specified above for preparing a pharmaceutical composition for the treatment of obstructive pulmonary diseases selected from among bronchial asthma, paediatric asthma, severe asthma, acute asthma attacks, chronic bronchitis and COPD (chronic obstructive pulmonary disease), while it is particularly preferable according to the invention to use them for preparing a pharmaceutical composition for the treatment of bronchial asthma and COPD.

It is also preferable to use the medicament combinations according to the invention for preparing a pharmaceutical composition for the treatment of pulmonary emphysema which has its origins in COPD (chronic obstructive pulmonary disease) or α1-proteinase inhibitor deficiency.

It is also preferable to use the medicament combinations according to the invention for preparing a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

It is also preferable to use the medicament combinations according to the invention for preparing a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic sclerodermy or sarcoidosis, granulomatoses, such as for example Boeck's disease, idiopathic interstitial pneumonia or idiopathic pulmonary fibrosis (IPF).

It is also preferable to use the medicament combinations according to the invention for preparing a pharmaceutical composition for the treatment of cystic fibrosis or mucoviscidosis.

It is also preferable to use the medicament combinations according to the invention for preparing a pharmaceutical composition for the treatment of bronchitis, such as for example bronchitis caused by bacterial or viral infection, allergic bronchitis and toxic bronchitis.

It is also preferable to use the medicament combinations according to the invention for preparing a pharmaceutical composition for the treatment of bronchiectasis.

It is also preferable to use the medicament combinations according to the invention for preparing a pharmaceutical composition for the treatment of ARDS (adult respiratory distress syndrome).

It is also preferable to use the medicament combinations according to the invention for preparing a pharmaceutical composition for the treatment of pulmonary oedema, for example toxic pulmonary oedema after aspiration or inhalation of toxic substances and foreign substances.

It is particularly preferable to use the compounds detailed above for preparing a pharmaceutical composition for the treatment of asthma or COPD. Also of particular importance is the above-mentioned use of medicament combinations according to the invention for preparing a pharmaceutical composition for once-a-day treatment of inflammatory and obstructive respiratory complaints, particularly for the once-a-day treatment of asthma or COPD.

The present invention also relates to the use of therapeutically effective amounts of an active substance of formula 1 in combination with therapeutically effective amounts of active substance 2 for preparing a pharmaceutical composition for the treatment of one of the above-mentioned diseases.

The present invention also relates to a process for treating one of the above-mentioned diseases, which is characterised in that therapeutically effective amounts of active substance of formula 1 are administered in combination with therapeutically effective amounts of active substance 2.

Within the scope of the medicament combinations according to the invention, for example, 0.1-1000 μg of a compound of formula 1 may be administered per single dose. Preferably, 1-500 μg, particularly preferably 3-100 μg of the compound of formula 1 are administered per single dose, while a dosage range of from 5-75 μg, preferably from 7-50 μg is preferred according to the invention. Particularly preferably, the pharmaceutical compositions according to the invention are administered in an amount such that 9-40 μg, particularly preferably 11-30 μg, more preferably 12-25 μg of the compound of formula 1 are administered per single dose . For example, and without restricting the present invention thereto, 5 μg, 7.5 μg, 10 μg, 12.5 μg, 15 μg, 17.5 μg, 20 μg, 22.5 μg, 25 μg, 27.5 μg, 30 μg, 32.5 μg, 35 μg, 37.5 μg, 40 μg, 42.5 μg, 45 μg, 47.5 μg, 50 μg, 52.5 μg, 55 μg, 57.5 μg, 60 μg, 62.5 μg, 65 μg, 67.5 μg, 70 μg, 72.5 μg or 75 μg of a compound of formula 1 may be administered per single dose.

The above-mentioned dosages relate to the compounds of formula 1 in the form of their free bases. If the compounds of formula 1 are administered in the form of their pharmaceutically acceptable acid addition salts, the skilled man can easily calculate the corresponding dosage ranges for the acid addition salts from the dosage ranges specified above, taking into account the molecular weight of the acids used. Particularly preferably, the compounds of formula 1 are administered in the above-mentioned dosage ranges in the form of the enantiomerically pure compounds, particularly preferably in the form of the R-enantiomers thereof.

If the compounds of formula 1 are administered in conjunction with an anticholinergic 2a, the amount of anticholinergic used will fluctuate considerably depending on the choice of active substance.

Without restricting the invention thereto, in the case of tiotropium 2a.1′ amounts of anticholinergic (2a.1′) may be administered such that each single dose contains 0.1-80 μg, preferably 0.5-60 μg, particularly preferably about 1-50 μg of 2a.1′. For example and without restricting the present invention thereto, 2.5 μg, 5 μg, 10 μg, 18 μg, 20 μg, 36 μg or 40 μg 2a.1′ may be administered per single dose. The corresponding amount of salt 2a.1 or of any hydrate or solvate used in each case can easily be calculated by the skilled man, depending on the choice of anion. If for example tiotropium bromide is used as the preferred tiotropium salt 2a.1 according to the invention, the amounts of the active substance 2a.1′ administered per single dose as specified by way of example hereinbefore correspond to the following amounts of 2a.1 administered per single dose: 3 μg, 6 μg, 12 μg, 21.7 μg, 24.1 μg, 43.3 μg and 48.1 μg 2a.1. In the case of tiotropium 2a.1′ the dosages specified above are preferably administered once or twice a day, while administration once a day is particularly preferred according to the invention.

Without restricting the invention thereto, in the case of the cation 2a.2′ amounts of anticholinergic (2a.2′) may be administered such that each single dose contains 1-500 μg, preferably 5-300 μg, particularly preferably 15-200 μg 2a.2′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.2′ may be administered per single dose. The corresponding amount of salt 2a.2 used in each case or of any hydrate or solvate used can easily be calculated by the skilled man, depending on the choice of anion. In the case of oxitropium 2a.2′ the dosages specified above are preferably administered one to four times a day, while administration two to three times a day is particularly preferred according to the invention.

Without restricting the invention thereto, in the case of the cation 2a.3′ amounts of anticholinergic (2a.3′) may be administered such that each single dose contains 1-500 μg, preferably 5-300 μg, particularly preferably 15-200 μg 2a.3′. For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.3′ may be administered per single dose. The corresponding amount of salt 2a.3 used in each case or of any hydrate or solvate used can easily be calculated by the skilled man, depending on the choice of anion. In the case of flutropium 2a.3′ the dosages specified above are preferably administered one to four times a day, while administration two to three times a day is particularly preferred according to the invention.

Without restricting the invention thereto, in the case of the cation 2a.4′ amounts of anticholinergic (2a.4′) may be administered such that each single dose contains 1-500 μg, preferably 5-300 μg, particularly preferably 20-200 μg 2a.4′. For example and without restricting the present invention thereto, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.4′ may be administered per single dose. The corresponding amount of salt 2a.4 used in each case or of any hydrate or solvate used can easily be calculated by the skilled man, depending on the choice of anion. In the case of ipratropium 2a.4′ the dosages specified above are preferably administered one to four times a day, while administration two to three times a day, more preferably three times a day, is particularly preferred according to the invention.

Without restricting the invention thereto, in the case of the cation 2a.5′ amounts of anticholinergic (2a.5′) may be administered such that each single dose contains 1-500 μg, preferably 5-300 μg, particularly preferably 15-200 μg . For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.5′ may be administered per single dose. The corresponding amount of salt 2a.5 used in each case or of any hydrate or solvate used can easily be calculated by the skilled man, depending on the choice of anion. In the case of glycopyrronium 2a.5′ the dosages specified above are preferably administered one to four times a day, while administration two to three times a day is particularly preferred according to the invention.

Without restricting the invention thereto, in the case of the cation 2a.6′ amounts of anticholinergic (2a.6′) may be administered such that each single dose contains 1000-6500 μg, preferably 2000-6000 μg, particularly preferably 3000-5500 μg, particularly preferably 4000-5000 μg 2a.6′. For example and without restricting the present invention thereto, 3500 μg, 3750 μg, 4000 μg, 4250 μg, 4500 μg, 4750 μg, or 5000 μg of 2a.6′ may be administered per single dose. The corresponding amount of salt 2a.6 used in each case or of any hydrate or solvate used can easily be calculated by the skilled man, depending on the choice of anion. In the case of trospiums 2a.6′ the dosages specified above are preferably administered one to four times a day, while administration two to three times a day is particularly preferred according to the invention.

Without restricting the invention thereto, in the case of the cation 2a.7′ amounts of anticholinergic (2a.7′) may be administered such that each single dose contains 50-1000 μg, preferably 100-800 μg, particularly preferably 200-700 μg, particularly preferably 300-600 μg 2a.7′. For example and without restricting the present invention thereto, 300 μg, 350 μg, 400 μg, 450 μg, 500 μg, 550 μg, or 600 μg of 2a.7′ may be administered per single dose. The corresponding amount of salt 2a.7 used in each case or of any hydrate or solvate used can easily be calculated by the skilled man, depending on the choice of anion. In the case of the cation 2a.7′ the dosages specified above are preferably administered one to three times a day, while administration once or twice a day, more preferably once a day, is particularly preferred according to the invention.

Without restricting the invention thereto, in the case of the cations 2a.940 and 2a.1040 , amounts of anticholinergic (2a.940 or 2a.1040 ) may be administered such that each single dose contains 1-500 μg, preferably 5-300 μg, particularly preferably 15-200 μg 2a.940 or 2a.1040 . For example and without restricting the present invention thereto, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.940 or 2a.1040 may be administered per single dose. The corresponding amount of salt 2a.940 or 2a.1040 or of any hydrate or solvate used in each case can easily be calculated by the skilled man, depending on the choice of anion. In the case of the cations 2a.940 or 2a.1040 the dosages specified above are preferably administered one to three times a day, while administration once or twice a day, more preferably once a day, is particularly preferred according to the invention.

Without restricting the invention thereto, in the case of the cations 2a.1140 to 2a.1340 amounts of anticholinergic (2a.1140 , 2a.1240 or 2a.1340 ) may be administered such that each single dose contains 1-500 μg, preferably 5-300 μg, particularly preferably 10-200 μg 2a.1140 , 2a.1240 or 2a.1340 . For example and without restricting the present invention thereto, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg or 200 μg of 2a.1140 , 2a.1240 or 2a.1340 may be administered per single dose . The corresponding amount of salt 2a.11, 2a.12 or 2a.13 or of any hydrate or solvate used in each case can easily be calculated by the skilled man, depending on the choice of anion. In the case of the cations 2a.11, 2a.12 or 2a.13 the dosages specified above are preferably administered one to three times a day, while administration once or twice a day, more preferably once a day, is particularly preferred according to the invention.

If the compounds of formula 1 are administered in combination with a PDE IV-inhibitor 2b, preferably about 1-10000 μg 2b are administered per single dose. Preferably, amounts of 2b are administered such that each single dose contains 10-5000 μg, preferably 50-2500 μg, particularly preferably 100-1000 μg of 2b . For example and without restricting the present invention thereto, 100 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg or 1000 μg of 2b may be administered per single dose. In the event that acid addition salts of 2b are used, the corresponding amount of salt used can easily be calculated by the skilled man from the values given hereinbefore, depending on the choice of acid.

If the compounds of formula I are administered in combination with a steroid 2c, preferably about 1-10000 μg of 2c are administered per single dose. Preferably, amouts of 2c are administered such that each single dose contains 5-5000 μg, preferably 5-2500 μg, particularly preferably 10-1000 μg of 2c . For example and without restricting the present invention thereto, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 115 μg, 120 μg, 125 μg, 130 μg, 135 μg, 140 μg, 145 μg, 150/μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 82 g, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg or 1000 kg of 2c may be administered per single dose. In the event that salts or derivatives of 2c are used, the corresponding amount of salt/derivative used can easily be calculated by the skilled man from the values given hereinbefore, depending on the choice of salt/derivative.

If the compounds of formula 1 are administered in combination with an LTD4-antagonist 2d, preferably about 0.01-500 mg 2d are administered per single dose . Preferably, amounts of 2d are administered such that each single dose contains 0.1-250 mg, preferably 0.5-100 mg, particularly preferably 1-50 mg of 2d . For example and without restricting the present invention thereto, 1 mg, 2.5 mg, 5 mg, 5.5 mg, 7 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg or 50 mg of 2d may be administered per single dose. In the event that acid addition salts, salts or derivatives of 2d are used, the corresponding amount of salt/derivative used can easily be calculated by the skilled man from the values given hereinbefore, depending on the choice of salt/derivative.

If the compounds of formula 1 are administered in combination with an EGFR-inhibitor 2e, preferably about 100-15000 μg of 2e are administered per single dose. Preferably, amounts of 2e are administered such that each single dose contains 500-1000 μg, preferably 750-8000 μg, particularly preferably 1000-7000 μg of 2e. For example and without restricting the present invention thereto, 1000 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3050 μg, 3100 μg, 3150 μg, 3200 μg, 3250 μg, 3300 μg, 3350 μg, 3400 μg, 3450 μg, 3500 μg, 3550 μg, 3600 μg, 3650 μg, 3700 μg, 3750 μg, 3800 μg, 3850 μg, 3900 μg, 3950 μg, 4000 μg, 4050 μg, 4100 μg, 4150 μg, 4200 μg, 4250 μg, 4300 μg, 4350 μg, 4400 μg, 4450 μg, 4500 μg, 4550 μg, 4600 μg, 4650 μg, 4700 μg, 4750 μg, 4800 μg, 4850 μg, 4900 μg, 4950 μg, 5000 μg, 5050 μg, 5100 μg, 5150 μg, 5200 μg, 5250 μg, 5300 μg, 5350 μg, 5400 μg, 5450 μg, 5500 μg, 5550 μg, 5600 μg, 5650 μg, 5700 μg, 5750 μg, 5800 μg, 5850 μg, 5900 μg, 5950 μg, 6000 μg, 6050 μg, 6100 μg, 6150 μg, 6200 μg, 6250 μg, 6300 μg, 6350 μg, 6400 μg, 6450 μg, 6500 μg, 6550 μg, 6600 μg, 6650 μg, 6700 μg, 6750 μg, 6800 μg, 6850 μg, 6900 μg, 6950 μg, or 7000 μg of 2e may be administered per single dose. In the event that acid addition salts of 2e are used, the corresponding amount of the salt used can easily be calculated by the skilled man from the values given hereinbefore, depending on the choice of acid.

The two active substance components 1 and 2 may be administered—together or separately—in each case by inhalation or by oral, parenteral or some other route, in known manner, in substantially conventional formulations such as for example plain or coated tablets, pills, granules, aerosols, syrups, emulsions, suspensions, powders and solutions, using inert, non-toxic, pharmaceutically suitable carriers or solvents.

Suitable preparations for administering the compounds of formula 1 and 2 include tablets, capsules, suppositories, solutions, powders, etc. The proportion of pharmaceutically active compound or compounds should be in the range from 0.05 to 90% by weight, preferably 0.1 to 50% by weight of the total composition. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations of active substances according to the invention may additionally contain a sweetener such as saccharine, cyclamate, glycerol or sugar and a flavour enhancer, e.g. a flavouring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules. Suitable suppositories may be made for example by mixing with carriers provided for this purpose, such as neutral fats or polyethyleneglycol or the derivatives thereof. Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

Preferably, even when the two components 1 and 2 are administered separately, at least component 1 is administered by inhalation. If component 1 is administered by inhalation, when the two active substances are taken separately, component 2 may also be administered for example by oral or parenteral route using formulations conventional in the art such as plain or coated tablets, pills, granules, aerosols, syrups, emulsions, suspensions, powders and solutions, using inert, non-toxic, pharmaceutically suitable carriers or solvents.

Preferably, however, the medicament combinations according to the invention are administered by inhalation by means of a single preparation containing both active substances 1 and 2 or by means of separate preparations each containing only one of the active substances 1 and 2, suitable for administration by inhalation.

Inhalable preparations include inhalable powders, propellant-containing metered dose aerosols or propellant-free inhalable solutions. Inhalable powders according to the invention containing the combination of active substances 1 and 2 may consist of the active substances on their own or of a mixture of the active substances with physiologically acceptable excipients. Within the scope of the present invention, the term propellant-free inhalable solutions also includes concentrates or sterile inhalable solutions ready for use. The preparations according to the invention may contain the combination of active substances 1 and 2 either together in one formulation or in two separate formulations. These formulations which may be used within the scope of the present invention are described in more detail in the next part of the specification.

A) Inhalable Powder Containing the Combinations of Active Substances According to the Invention:

The inhalable powders according to the invention may contain 1 and 2 either on their own or in admixture with suitable physiologically acceptable excipients. If the active substances 1 and 2 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose, trehalose), oligo- and polysaccharides (e.g. dextrans), polyalcohols (e.g. sorbitol, mannitol, xylitol), salts (e.g. sodium chloride, calcium carbonate) or mixtures of these excipients with one another. Preferably, mono- or disaccharides are used, while the use of lactose, trehalose or glucose is preferred, particularly, but not exclusively, in the form of their hydrates.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with an average particle size of 1 to 9 μm to the excipients mentioned above. These finer excipients are also selected from the group of possible excipients listed hereinbefore. Finally, in order to prepare the inhalable powders according to the invention, micronised active substance 1 and 2, preferably with an average particle size of 0.5 to 10 μm, more preferably from 1 to 6 μm, is added to the excipient mixture. Processes for producing the inhalable powders according to the invention by grinding and micronising and finally mixing the ingredients together are known from the prior art. The inhalable powders according to the invention may be prepared and administered either in the form of a single powder mixture which contains both 1 and 2 or in the form of separate inhalable powders which contain only 1 or 2.

The inhalable powders according to the invention may be administered using inhalers known from the prior art. Inhalable powders according to the invention which contain a physiologically acceptable excipient in addition to 1 and 2 may be administered, for example, by means of inhalers which deliver a single dose from a supply using a measuring chamber as described in U.S. Pat. No. 4,570,630A, or by other means as described in DE 36 25 685 A. The inhalable powders according to the invention which contain 1 and 2 optionally in conjunction with a physiologically acceptable excipient may be administered, for example, using the inhaler known by the name Turbuhaler® or using inhalers as disclosed for example in EP 237507 A. Preferably, the inhalable powders according to the invention which contain physiologically acceptable excipient in addition to 1 and 2 are packed into capsules (to produce so-called inhalettes) which are used in inhalers as described, for example, in WO 94/28958.

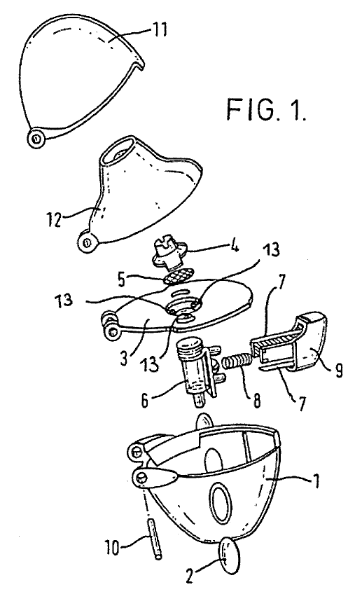

A particularly preferred inhaler for using the pharmaceutical combination according to the invention in inhalettes is shown in FIG. 1.

This inhaler (Handihaler®) for inhaling powdered pharmaceutical compositions from capsules is characterised by a housing 1 containing two windows 2, a deck 3 in which there are air inlet ports and which is provided with a screen 5 secured by a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, and air through-holes 13 for adjusting the flow resistance.

If the inhalable powders according to the invention are to be packaged in capsules, in accordance with the preferred method of administration described above, the capsules should preferably contain from 1 to 30 mg each. According to the invention they contain either together or separately the dosages per single dose specified for 1 and 2 hereinbefore.

B) Propellant Gas-Driven Inhalation Aerosols Containing the Combinations of Active Substances According to the Invention:

Inhalation aerosols containing propellant gas according to the invention may contain substances 1 and 2 dissolved in the propellant gas or in dispersed form. 1 and 2 may be present in separate formulations or in a single preparation, in which 1 and 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane or isobutane and halohydrocarbons such as preferably chlorinated and fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG11, TG12, TG134a (1,1,1,2-tetrafluoroethane), TG227 (1,1,1,2,3,3,3-heptafluoropropane) and mixtures thereof, the propellant gases TG134a, TG227 and mixtures thereof being preferred.

The propellant-driven inhalation aerosols according to the invention may also contain other ingredients such as co-solvents, stabilisers, surfactants, antioxidants, lubricants and pH adjusters. All these ingredients are known in the art.

The inhalation aerosols containing propellant gas according to the invention may contain up to 5 wt.-% of active substance 1 and/or 2. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of active substance 1 and/or 2.

If the active substances 1 and/or 2 are present in dispersed form, the particles of active substance preferably have an average particle size of up to 10 μm, preferably from 0.1 to 6 μm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention.

The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

C) Propellant-Free Inhalable Solutions or Suspensions Containing the Combinations of Active Substances 1 and 2 According to the Invention:

Propellant-free inhalable solutions according to the invention contain for example aqueous or alcoholic, preferably ethanolic solvents, possibly ethanolic solvents in admixture with aqueous solvents. In the case of aqueous/ethanolic solvent mixtures the relative proportion of ethanol to water is not restricted, but the maximum limit is up to 70 percent by volume, more particularly up to 60 percent by volume of ethanol. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid, etc. Preferred inorganic acids are hydrochloric acid and sulphuric acid. It is also possible to use the acids which have already formed an acid addition salt with one of the active substances. Of the organic acids, ascorbic acid, fumaric acid and citric acid are preferred. If desired, mixtures of the above acids may also be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

According to the invention, the addition of edetic acid (EDTA) or one of the known salts thereof, sodium edetate, as stabiliser or complexing agent is unnecessary in the present formulation. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the combination of active substances 1 and 2, only benzalkonium chloride and sodium edetate. In another preferred embodiment, no sodium edetate is present.

The propellant-free inhalable solutions according to the invention are administered in particular using inhalers of the kind which are capable of nebulising a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 10 and 30 μL of active substance solution can be nebulised in preferably one spray action to form an aerosol with an average particle size of less than 20 μm, preferably less than 10 μm, such that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulisers (devices) described therein are known by the name Respimat®.

The above-mentioned examples of the active substances 2 are known in the art. The compounds of formula 1 by contrast are not known in the art.

The examples of synthesis described hereinafter serve to illustrate possible methods of synthesising the new compounds of formula 1. However, they are intended only as examples of procedures as an illustration of the invention without restricting the invention to the subject-matter described by way of example.

### EXAMPLE 1.1

### 6-hydroxy-8-{1-hydroxy-2-[2-(4-methoxy-phenyl)-1,1-dimethyl-ethylamino]-ethyl}-4H-benzo[1,4]oxazin-3-one

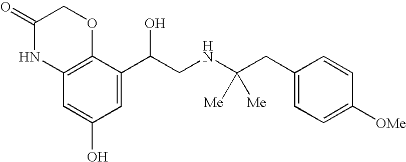 a) 8-{2-[1,1-dimethyl-2-(4-methoxy-phenyl)-ethylamino]-1-hydroxy-ethyl}-6-benzyloxy-4H-benzo[1,4]oxazin-3-one

7.5 g (6-benzyloxy-4H-benzo[1,4]oxazin-3-one)-glyoxalhydrate are added at 70° C. to a solution of 3.6 g 1,1-dimethyl-2-(4-methoxyphenyl)-ethylamine in 100 mL ethanol and the mixture is stirred for 15 minutes. Then 1 g sodium borohydride is added within 30 minutes at 10 to 20° C. It is stirred for one hour, combined with 10 mL acetone and stirred for a further 30 minutes. The reaction mixture is diluted with 150 mL ethylacetate, washed with water, dried with sodium sulphate and evaporated down. The residue is dissolved in 50 mL methanol and 100 mL ethyl acetate and acidified with conc. hydrochloric acid. After the addition of 100 mL diethyl ether the product crystallises out. The crystals are filtered off, washed and recrystallised from 50 mL ethanol.

Yield: 7 g (68%; hydrochloride); m.p.=232-234° C.

b) 8-{2-[1,1-dimethyl-2-(4-methoxy-phenyl)-ethylamino]-1-hydroxy-ethyl}-6-hydroxy-4H-benzo[1,4oxazin-3-one

6.8 g of the benzyl compound obtained previously are hydrogenated in 125 mL methanol with the addition of 1 g palladium on charcoal (5%) at ambient temperature and normal pressure. The catalyst is filtered off and the filtrate freed from solvent. After recrystallisation of the residue from 50 mL acetone and some water a solid is obtained which is filtered off and washed.

Yield: 5.0 g (89%; hydrochloride); m.p.=155-160° C.

The (R)- and (S)-enantiomers of Example 1.1 may be obtained from the racemate, for example, by chiral HPLC (e.g. column: Chirobiotic T made by Messrs Astec, 250×22.1 mm, 5 μm). Methanol with 0.05% triethylamine and 0.05% acetic acid may be used as the mobile phase. The retention times of the R- or S-enantiomer are between 35 and 65 min depending on the flow, while the R-enantiomer is eluted first. Of outstanding importance according to the invention is the R-enantiomer of Example 1.1.

### EXAMPLE 1.2

### 6-hydroxy-8-{1-hydroxy-2-[2-(4-phenoxyethyl-acetate)-1,1-dimethyl-ethylamino]-ethyl}-4H-benzo[1,4]oxazin-3-one

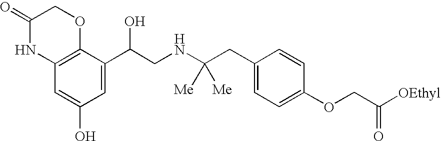 a) 8-{2-[1,1-dimethyl-2-(4-phenoxyethyl-acetate)-ethylamino]-1-hydroxy-ethyl}-6-benzyloxy-4H-benzo[1,4]oxazin-3-one

Analogously to the method described in Example 1.1a) the title compound is obtained from 15 g (6-benzyloxy-4H-benzo[1,4]oxazin-3-one)-glyoxalhydrate and 11.8 g 1,1-dimethyl-2-(4-phenoxyethyl-acetate)-ethylamine hydrochloride.

Yield: 16.5 g (69%, hydrochloride); m.p.=212-214° C.

b) 8-{2-[1,1-dimethyl-2-(4-phenoxyethyl-acetate)-ethylamino]-1-hydroxy-ethyl}-6-hydroxy-4H-benzo[1,4]oxazin-3-one

8 g of the benzylalcohol obtained previously are dissolved in 100 mL ethanol, 100 mL methanol and 10 mL water and hydrogenated in the presence of 1 g palladium on charcoal (5%). After the absorption of the theoretically calculated amount of hydrogen the catalyst is filtered off and the filtrate is evaporated down. The product that crystallises out on distillation of the solvent is suction filtered and washed.

Yield: 5.5 g (81%; hydrochloride); m.p.=137-140° C.

The (R)- and (S)-enantiomers of this embodiment may be obtained by separation of the racemate analogously to common methods known in the art.

### EXAMPLE 1.3

### 6-hydroxy-8-{1-hydroxy-2-[2-(4-phenoxy-acetic acid)-1,1-dimethyl-ethylamino]-ethyl}-4H-benzo[1,4]oxazin-3-one

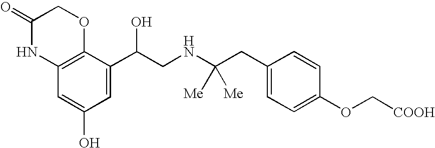

11 g of 8-{2-[1,1-dimethyl-2-(4-phenoxyethyl-acetate)-ethylamino]-1-hydroxy-ethyl}-6-benzyloxy-4H-benzo[1,4]oxazin-3-one hydrochloride (Example 4a) are dissolved in 125 mL methanol and hydrogenated in the presence of 1 g palladium on charcoal (5%). After the absorption of the theoretically calculated amount of hydrogen the catalyst is filtered off. 2.6 g sodium hydroxide dissolved in 20 mL water are added to the filtrate. The mixture is refluxed for 30 minutes, the methanol is distilled off and 10 mL water, 20 mL n-butanol and 3.9 mL acetic acid. The solid precipitated is suction filtered and washed with diethyl ether.

Yield: 7 g (87%). The hydrochloride is obtained by recrystallisation from 0.5 molar hydrochloric acid. M.p.=152° C.

The (R)- and (S)-enantiomers of this embodiment may be obtained by separation of the racemate analogously to common methods known in the art.

### EXAMPLE 1.4

### 8-{2-[1,1-dimethyl-2-(2,4,6-trimethylphenyl)-ethylamino]-1-hydroxy-ethyl}-6-hydroxy-4H-benzo[1,4]oxazin-3-one

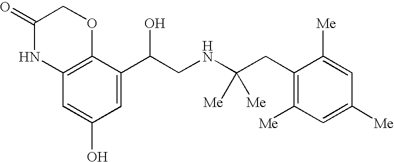 a) 1-(6-benzyloxy-4H-benzo[1,4]oxazin-3-one)-2-[1,1-dimethyl-2-(2,4,6-trimethylphenyl)-ethylimino]-ethanone

7.2 g (6-benzyloxy-4H-benzo[1,4]oxazin-3-one)-glyoxalhydrate and 3.6 g 1,1-dimethyl-2-(2,4,6-trimethylphenyl)-ethylamine are heated to 70° C. for one hour in 100 mL ethanol. After cooling the precipitated crystals are filtered off and washed with ethanol and diethyl ether. Yield: 8.6 g (94%); m.p.=175° C.

b) 8-{2-[1,1-dimethyl-2-(2,4,6-trimethylphenyl)-ethylamino]-1-hydroxy-ethyl}-6-benzyloxy-4H-benzo[1,4]oxazin-3-one 8.6 g of the Schiff base obtained according to the prescribed method 1.4a) are dissolved in 100 mL ethanol and 20 mL THF, combined with 0.7 g sodium borohydride within 30 min at 10-20° C. and stirred for one hour. After the addition of 10 mL acetone the mixture is stirred for 30 minutes and then diluted with ethyl acetate and water. The product that crystallises out during acidification with conc. hydrochloric acid is filtered off and washed.

Yield: 7.4 g (80%, hydrochloride); m.p.=235° C. (decomposition).

c) 8-{2-[1,1-dimethyl-2-(2,4,6-trimethylphenyl)-ethylamino]-1-hydroxy-ethyl}-6-hydroxy-4H-benzo[1,4]oxazin-3-one

7.4 g of the benzyl compound obtained in Step b) are hydrogenated in 125 mL methanol with the addition of 1 g palladium on charcoal (5%) at ambient temperature and normal pressure. Then the catalyst is filtered off and the filtrate evaporated down. The product that crystallises out on the addition of acetone is suction filtered and washed with acetone and diethyl ether. Yield: 5 g (78%, hydrochloride); m.p. 160° C. (decomposition).

The (R)- and (S)-enantiomers of this embodiment may be obtained by separation of the racemate analogously to common methods known in the art.

### EXAMPLE 1.5

### 6-hydroxy-8-{1-hydroxy-2-[2-(4-hydroxy-phenyl)-1,1-dimethyl-ethylamino]-ethyl}-4H-benzo[1,4]oxazin-3-one

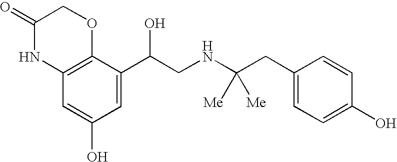 a) 8-{2-[1,1-dimethyl-2-(4-hydroxy-phenyl)-ethylamino]-1-hydroxy-ethyl}-6-benzyloxy-4H-benzo[1,4]oxazin-3-one

The title compound is prepared from 10 g (6-benzyloxy-4H-benzo[1,4]oxazin-3-one)-glyoxalhydrate and 4.6 g 1,1-dimethyl-2-(4-hydroxy-phenyl)-ethylamine analogously to the procedure laid down for Example 1.1a).

Yield: 9.0 g (64%, hydrochloride); m.p.=255-258° C.

b) 8-{2-[1,1-dimethyl-2-(4-hydroxy-phenyl)-ethylamino]-1-hydroxy-ethyl}-6-hydroxy-4H-benzo[1,4]oxazin-3-one

5.7 g of the coupling product obtained previously are hydrogenated in the presence of 0.6 g palladium on charcoal (5%) in 100 mL methanol. After the absorption of the theoretically calculated amount of hydrogen the catalyst is filtered off and the filtrate freed from solvent. The residue is dissolved in ethanol with heating and then combined with diethyl ether. The product precipitated is suction filtered and recrystallised once from water.

Yield: 3.6 g (72%, hydrochloride); m.p.=159-162° C.

The (R)- and (S)-enantiomers of this embodiment may be obtained by separation of the racemate analogously to common methods known in the art.

### EXAMPLE 1.6

### 6-hydroxy-8-{1-hydroxy-2-[2-(4-isopropyl-phenyl)-1,1dimethyl-ethylamino]-ethyl}-4H-benzo[1,4]oxazin-3-one

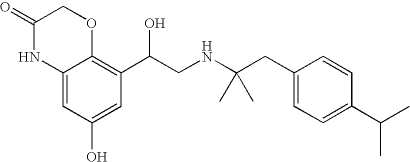 a) 1-(4-isopropyl-phenyl)-2-methyl-propan-2-ol

The reaction of a Grignard compound, prepared from 20 g (119 mmol) 4-isopropylbenzyl chloride, with 11.4 ml (155 mmol) acetone yields the target compound as a colourless oil. Yield: 13.0 g (57%); mass spectrometry: [M+H]⁺=193.

b) N-[2-(4-isopropyl-phenyl)-1,1-dimethyl-ethyl]-acetamide

A Ritter reaction is carried out with 10.2 g (53 mmol) 1-(4-isopropyl-phenyl)-2-methyl-propan-2-ol in the manner described for Example 1.7b). The reaction mixture is poured onto ice water and made alkaline with sodium hydroxide solution, during which time a solid is precipitated. This is suction filtered and dried.

Yield: 9.90 g (80%); mass spectrometry: [M+H]⁺=234.

c) 2-(4-isopropyl-phenyl)-1,1-dimethyl-ethylamine

Reaction of 9.80 g (42 mmol) N-[2-(4-isopropyl-phenyl)-1,1-dimethyl-ethyl]-acetamide analogously to the procedure laid down for Example 1.7c).

Yield: 7.00 g (71%, hydrochloride); m.p. 202-206° C.

d) 6-benzyloxy-8-{1-hydroxy-2-[2-(4-isopropyl-phenyl)-1,1-dimethyl-ethylamino]-ethyl}-4H-benzo[1,4]oxazin-3-one

2.18 g (6.1 mmol) benzyloxy-8-(2-ethoxy-2-hydroxy-acetyl)-4H-benzo[1,4]oxazin-3-one and 1.1 g (5.8 mmol) 2-(4-isopropyl-phenyl)-1,1-dimethyl-ethylamine are stirred for one hour at 50-80° C. in 40 mL ethanol. After cooling to ambient temperature 0.24 g (6.3 mmol) sodium borohydride are added. The mixture is stirred for one hour, diluted with 5 mL acetone and stirred for a further 30 minutes. The reaction mixture is acidified with hydrochloric acid, combined with 100 mL water and 80 mL ethyl acetate and made alkaline with ammonia. The organic phase is separated off, dried with sodium sulphate and freed from solvent. The residue is dissolved in 20 mL ethyl acetate and 10 mL water, acidified with conc. hydrochloric acid and diluted with diethyl ether. After the addition of a crystallisation aid the precipitated solid is suction filtered and washed. White solid.

Yield: 1.7 g (52%, hydrochloride); m.p. 220-222° C.

e) 6-hydroxy-8-{1-hydroxy-2-[2-(4-isopropyl-phenyl)-1,1dimethyl-ethylamino]-ethyl}-4H-benzo[1,4]oxazin-3-one

1.6 g (3.0 mmol) 6-benzyloxy-8-{1-hydroxy-2-[2-(4-isopropyl-phenyl)-1,1-dimethyl-ethylamino]-ethyl}-4H-benzo[1,4]oxazin-3-one are dissolved in methanol and hydrogenated with palladium on charcoal as catalyst at normal pressure and ambient temperature. The catalyst is suction filtered, the solvent is distilled off and the residue is recrystallised from isopropanol. White solid.

Yield: 1.1 g (85%, hydrochloride); m.p. 248-250° C.; mass spectrometry: [M+H]⁺=399.

The (R)- and (S)-enantiomers of this embodiment may be obtained by separation of the racemate analogously to common methods known in the art.

### EXAMPLE 1.7

### 8-{2-[2-(4-ethyl-phenyl)-1,1-dimethyl-ethylamino]-1-hydroxy-ethyl}-6-hydroxy-4H-benzo[1,4]oxazin-3-one

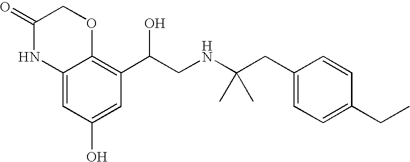 a) 1-(4-ethyl-phenyl)-2-methyl-propan-2-ol

14.8 g (90 mmol) 1-(4-ethyl-phenyl)-propan-2-one, dissolved in diethyl ether, are added dropwise to 39 mL of a 3 molar solution of methylmagnesium bromide in diethyl ether, while being cooled with the ice bath, such that the temperature does not exceed 30° C. After the addition has ended the reaction mixture is left to reflux for 1.5 hours and then hydrolysed with 10% ammonium chloride solution. After separation of the organic phase the aqueous phase is extracted with diethyl ether. The combined ether phases are washed with water, dried with sodium sulphate and evaporated down. The oil thus obtained is further reacted directly. Yield: 15.5 g (90%).

b) N-[2-(4-ethyl-phenyl)-1,1-dimethyl-ethyl]-acetamide

6.2 mL conc. sulphuric acid are added dropwise within 15 minutes to 15.5 g (87 mmol) 1-(4-ethyl-phenyl)-2-methyl-propan-2-ol in 4.8 mL (91 mmol) acetonitrile and 15 mL glacial acetic acid, while the temperature rises to 65° C. Then the mixture is stirred for one hour, diluted with ice water and made alkaline with conc. sodium hydroxide solution. After further stirring for 30 minutes the precipitated solid is suction filtered and washed with water. The crude product is dissolved in ethyl acetate, dried with sodium sulphate and evaporated down. The oil remaining is combined with petroleum ether, during which time a solid is precipitated, which is filtered off and dried. Yield: 16.3 g (85%); m.p. 90-92° C.

c) 2-(4-ethyl-phenyl)-1,1-dimethyl-ethylamine

16.3 g (74 mmol) N-[2-(4-ethyl-phenyl)-1,1-dimethyl-ethyl]-acetamide and 8.0 g potassiumhydroxid are through 15 hours in 60 mL ethyleneglycol at reflux temperature heated. The reaction mixture is combined with ice water and extracted three times with diethyl ether. The combined organic phases are washed with water, dried with sodium sulphate and freed from solvent. To produce the hydrochlorides, the crude product is dissolved in acetonitrile and successively combined with ethereal hydrochloric acid and diethyl ether. The solid precipitated is suction filtered and dried. Yield: 11.0 g (69%, hydrochloride); m.p. 165-167° C.

d) 6-benzyloxy-8-{2-[2-(4-ethyl-phenyl)-1,1-dimethyl-ethylamino]-1-hydroxy-ethyl}-4H-benzo[1,4]oxazin-3-one

The target compound is prepared analogously to the procedure laid down for Example 1.6d) from 2.14 g (6.0 mmol) 6-benzyloxy-8-(2-ethoxy-2-hydroxy-acetyl)-4H-benzo[1,4]oxazin-3-one and 1.0 g (5.6 mmol) 2-(4-ethyl-phenyl)-1,1-dimethyl-ethylamine. White solid. Yield: 1.7 g (54%, hydrochloride); m.p. 210-214° C.

e) 8-{2-[2-(4-ethyl-phenyl)-1,1-dimethyl-ethylamino]-1-hydroxy-ethyl}-6-hydroxy-4H-benzo[1,4]oxazin-3-one

The hydrogenolysis of 1.45 g (2.75 mmol) 6-benzyloxy-8-{2-[2-(4-ethyl-phenyl)-1,1-dimethyl-ethylamino]-1-hydroxy-ethyl}-4H-benzo[1,4]oxazin-3-one according to the prescribed method for Example 1.6e) yields the target compound in the form of a white solid. Yield: 1.07 g (92%; hydrochloride); m.p. 266-269° C.; mass spectrometry: [M+H]⁺=385.

The (R)- and (S)-enantiomers of this embodiment may be obtained by separation of the racemate analogously to common methods known in the art.

### EXAMPLE 1.8

### 8-{2-[2-(4-fluoro-3-methyl-phenyl)-1,1-dimethyl-ethylamino]-1-hydroxy-ethyl}-6-hydroxy-4H-benzo[1,4]oxazin-3-one

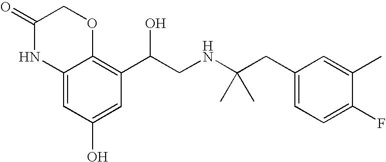 a) 1-fluoro-2-methyl-4-(2-methyl-propenyl)-benzene

100 mL of a 0.5 molar solution of 4-fluoro-3-methyl-phenylmagnesium bromide in THF are combined within 30 minutes with 4.7 mL (50 mmol) isopropylaldehyde, while the temperature rises to 45° C. The mixture is stirred for 30 minutes, refluxed for 1 hour and then hydrolysed with 10% ammonium chloride solution. After separation of the organic phase the mixture is extracted with diethyl ether. The organic phases are combined, dried and evaporated down. The alcohol thus obtained is dissolved in 100 mL toluene, combined with 1 g p-toluenesulphonic acid monohydrate and refluxed for three hours using the water separator. The reaction mixture is poured onto water and made alkaline with conc. sodium hydroxide solution. After separation of the organic phase the latter is washed with water, dried with sodium sulphate and freed from solvent. Fractional distillation of the residue yields the product in the form of a colourless liquid (b.p. 80-85° C./10 mbar).

Yield: 4.1 g (50%).

b) N-[2-(4-fluoro-3-methyl-phenyl)-1,1-dimethyl-ethyl]-formamide

4.9 mL conc. sulphuric acid are added dropwise at 5-15° C. to 1.5 g (31 mmol) sodium cyanide in 5 mL glacial acetic acid. Then the mixture is combined with 3.9 g (24 mmol) 1-fluoro-2-methyl-4-(2-methyl-propenyl)-benzene, dissolved in 10 mL glacial acetic acid, and stirred for 1 hour at 50-60° C. The reaction mixture is diluted with ice water, made alkaline with conc. sodium hydroxide solution and extracted with dichloromethane. The organic phase is dried with sodium sulphate and freed from the solvent in vacuo. The light yellow oil thus obtained is further reacted directly. Yield: 4.3 g (87%).

c) 2-(4-fluoro-3-methyl-phenyl)-1,1-dimethyl-ethylamine

4.3 g (20.6 mmol) N-[2-(4-fluoro-3-methyl-phenyl)-1,1-dimethyl-ethyl]-formamide, 20 mL conc. hydrochloric acid and 20 mL water are refluxed for 2 hours. The reaction mixture is diluted with water, made alkaline with conc. sodium hydroxide solution and extracted with dichloromethane. The organic phases are dried with sodium sulphate and evaporated down. The residue is dissolved in ethyl acetate, combined with ethereal hydrochloric acid and cooled. The crystals precipitated are suction filtered and washed with diethyl ether and dried. White solid.

Yield: 3.9 g (87%, hydrochloride); m.p. 196-198° C.

d) 6-benzyloxy-8-{2-[2-(4-fluoro-3-methyl-phenyl)-1,1-dimethyl-ethylamino]-1-hydroxy-ethyl}-4H-benzo[1,4]oxazin-3-one

1.10 g (3.1 mmol) benzyloxy-8-(2-ethoxy-2-hydroxy-acetyl)-4H-benzo[1,4]oxazin-3-one and 0.50 g (2.8 mmol) 2-(4-fluoro-3-methyl-phenyl)-1,1-dimethyl-ethylamine are reacted and worked up analogously to the procedure laid down for Example 1.6d). White solid.

Yield: 0.75 g (47%, hydrochloride); m.p. 228-230° C.

e) 8-{2-[2-(4-fluoro-3-methyl-phenyl)-1,1-dimethyl-ethylamino]-1-hydroxy-ethyl}-6-hydroxy-4H-benzo[1,4]oxazin-3-one

The hydrogenation of 0.70 g (1.4 mmol) 6-benzyloxy-8-{2-[2-(4-fluoro-3-methyl-phenyl)-1,1-dimethyl-ethylamino]-1-hydroxy-ethyl}-4H-benzo[1,4]oxazin-3-one yields the target compound as a white solid.

Yield: 0.50 g (87%, hydrochloride); m.p. 278-280° C.; mass spectroscopy: [M+H]⁺=389.

The (R)- and (S)-enantiomers of this embodiment may be obtained by separation of the racemate analogously to common methods known in the art.

### EXAMPLE 1.9

### 8-{2-[2-(4-fluoro-2-methyl-phenyl)-1,1-dimethyl-ethylamino]-1-hydroxy-ethyl}-6-hydroxy-4H-benzo[1,4]oxazin-3-one

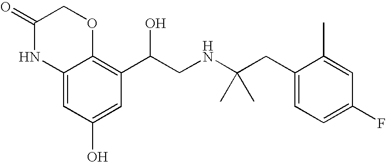 a) 1-(4-fluoro-2-methyl-phenyl)-2-methyl-propyl acetate

500 mL of a 0.5 molar solution of 4-fluoro-6-methylphenylmagnesium bromide and 23.2 mL (260 mmol) isopropylaldehyde are reacted analogously to Example 1.8a). After hydrolysis with 10% ammonium chloride solution the aqueous phase is separated off and extracted with diethyl ether. The combined organic phases are dried with sodium sulphate and evaporated down. The alcohol thus obtained is then dissolved in 50 mL acetic anhydride, combined with 1 mL conc. sulphuric acid and stirred for three hours at reflux temperature. Then the reaction mixture is poured onto water, stirred for a further hour and made alkaline. It is extracted with dichloromethane, the organic phases are dried with sodium sulphate and the solvents are distilled off. Fractional distillation of the residue yields the product in the form of a colourless liquid (b.p. 105-110° C./8 mbar).

Yield: 29.0 g (52%).

b) N-[2-(4-fluoro-2-methyl-phenyl)-1,1-dimethyl-ethyl]-formamide

29.0 g (130 mmol) 1-(4-fluoro-2-methyl-phenyl)-2-methyl-propyl acetate are reacted and worked up analogously to the procedure laid down for Example 1.8b). Yellow oil. Yield: 27.0 g (99%).

c) 2-(4-fluoro-2-methyl-phenyl)-1,1-dimethyl-ethylamine

In order to prepare the amine 27.0 g (130 mmol) N-[2-(4-fluoro-2-methyl-phenyl)-1,1-dimethyl-ethyl]-formamide are reacted as described in the procedure laid down for Example 1.8c). White solid. Yield: 15.5 g (55%, hydrochloride); m.p. 277-280° C.

d) 6-benzyloxy-8-{2-[2-[4-fluoro-2-methyl-phenyl)-1,1-dimethyl-ethylamino]-1-hydroxy-ethyl}-4H-benzo[1,4]oxazin-3-one

Prepared analogously to the procedure laid down for Example 1.6d) from 0.95 g (2.66 mmol) benzyloxy-8-(2-ethoxy-2-hydroxy-acetyl)-4H-benzo[1,4]oxazin-3-one and 0.43 g (2.37 mmol) 2-(4-fluoro-2-methyl-phenyl)-1,1-dimethyl-ethylamine.

Yield: 0.75 g (55%, hydrochloride); m.p. 233-236° C.

e) 8-{2-[2-(4-fluoro-2-methyl-phenyl)-1,1-dimethyl-ethylamino]-1-hydroxy-ethyl}-6-hydroxy-4H-benzo[1,4]oxazin-3-one

The debenzylation of 0.70 g (1.36 mmol) 6-benzyloxy-8-{2-[2-[4-fluoro-2-methyl-phenyl)-1,1-dimethyl-ethylamino]-1-hydroxy-ethyl}-4H-benzo[1,4]oxazin-3-one yields the target compound in the form of a white solid.

Yield: 0.50 g (87%, hydrochloride); m.p. 278-280° C.; mass spectroscopy: [M+H]⁺=389.

The (R)- and (S)-enantiomers of this embodiment may be obtained by separation of the racemate analogously to common methods known in the art.

### EXAMPLE 1.10

### 8-{2-[2-(2,4-difluoro-phenyl)-1,1-dimethyl-ethylamino]-1-hydroxy-ethyl}-6-hydroxy-4H-benzo[1,4]oxazin-3-one

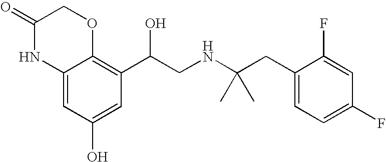 a) 1-(2,4-difluoro-phenyl)-2-methyl-propan-2-ol

11.0 mL acetone, diluted with 50 mL diethyl ether, are added dropwise within 20 minutes to a solution of 500 mL of 0.25 molar 2,4-difluorobenzylmagnesium bromide in diethyl ether. Then the mixture is stirred for 1.5 hours at reflux temperature and then hydrolysed with 10% ammonium chloride solution. The ether phase is separated off, washed with water, dried with sodium sulphate and evaporated down. The fractional distillation of the residues yields the alcohol as a colourless liquid (b.p. 70-73° C./2 mmbar).

Yield: 20.0 g (86%).

b) N-[2-(2,4-difluoro-phenyl]-1,1-dimethyl-ethyl]-formamide

Ritter reaction with 20 g (110 mmol) 1-(2,4-difluoro-phenyl)-2-methyl-propan-2-ol according to the process described for Example 1.8b). Yellow oil. Yield: 22.0 g (94%).

c) 2-(2,4-difluoro-phenyl)-1,1-dimethyl-ethylamine

Reaction of 22.0 g (100 mmol) N-[2-(2,4-difluoro-phenyl]-1,1-dimethyl-ethyl]-formamide analogously to the procedure laid down for Example 1.8c).

Yield: 16.0 g (72%, hydrochloride); m.p. 201-203° C.

d) 6-benzyloxy-8-{2-[2-(2,4-difluoro-phenyl)-1,1-dimethyl-ethylamino]-1-hydroxy-ethyl}-4H-benzo[1,4]oxazin-3-one

Reaction of 0.89 g (2.49 mmol) benzyloxy-8-(2-ethoxy-2-hydroxy-acetyl)-4H-benzo[1,4]oxazin-3-one and 0.40 g (2.16 mmol) 2-(2,4-difluoro-phenyl)-1,1-dimethyl-ethylamine in the manner described for Example 1.6d).

Yield: 0.80 g (62%, hydrochloride); m.p. 245-247° C.

e) 8-{2-[2-(2,4-difluoro-phenyl)-1,1-dimethyl-ethylamino]-1-hydroxy-ethyl}-6-hydroxy-4H-benzo[1,4]oxazin-3-one

The hydrogenolysis of 0.70 g (1.35 mmol) 6-benzyloxy-8-{2-[2-(2,4-difluoro-phenyl)-1,1-dimethyl-ethylamino]-1-hydroxy-ethyl}-4H-benzo[1,4]oxazin-3-one yields the target compound as a white solid.

Yield: 0.48 g (83%, hydrochloride); m.p. 279-280° C.; mass spectroscopy: [M+H]⁺=393.

The (R)- and (S)-enantiomers of this embodiment may be obtained by separation of the racemate analogously to common methods known in the art.

### EXAMPLE 1.11

### 8-{2-[2-(3,5-difluoro-phenyl)-1,1-dimethyl-ethylamino]-1-hydroxy-ethyl}-6-hydroxy-4H-benzo[1,4]oxazin-3-one

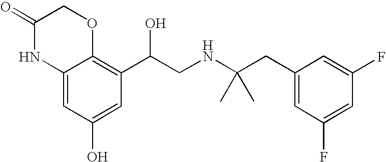 a) 1-(3,5-difluoro-phenyl)-2-methyl-propan-2-ol

The target compound is obtained by reacting a Grignard compound, prepared from 25.0 g (121 mmol) 3,5-difluorobenzylbromide, with 12.6 mL (171 mmol) acetone. Yellow oil.

Yield: 13.5 g (60%).

b) 2-(3,5-difluoro-phenyl)-1,1-dimethyl-ethylamine

The Ritter reaction of 5.5 g (29.5 mmol) 1-(3,5-difluoro-phenyl)-2-methyl-propan-2-ol and 1.8 g sodium cyanide yields 7.0 g formamide, which is treated with hydrochloric acid in order to cleave the formyl group. Light yellow oil. Yield: 4.6 g (75%).

c) 6-benzyloxy-8-{2-[2-(3,5-difluoro-phenyl)-1,1-dimethyl-ethylamino]-1-hydroxy-ethyl}-4H-benzo[1,4]oxazin-3-one

Prepared from 1.73 g (4.84 mmol) benzyloxy-8-(2-ethoxy-2-hydroxy-acetyl)-4H-benzo[1,4]oxazin-3-one and 0.80 g (4.32 mmol) 2-(3,5-difluoro-phenyl)-1,1-dimethyl-ethylamine in the conventional manner.

Yield: 1.50 g (58%, hydrochloride); m.p. 240-244° C.

d) 8-{2-[2-(3,5-difluoro-phenyl)-1,1-dimethyl-ethylamino]-1-hydroxy-ethyl}-6-hydroxy-4H-benzo[1,4]oxazin-3-one

Hydrogenolysis of 1.30 g (2.43 mmol) 6-benzyloxy-8-{2-[2-(3,5-difluoro-phenyl)-1,1-dimethyl-ethylamino]-1-hydroxy-ethyl}-4H-benzo[1,4]oxazin-3-one yields the target compound as a white solid.

Yield: 0.90 g (86%, hydrochloride); m.p. 150-158° C.; mass spectroscopy: [M+H]⁺=393.

The (R)- and (S)-enantiomers of this embodiment may be obtained by separation of the racemate analogously to common methods known in the art.

### EXAMPLE 1.12

### 8-{2-[2-(4-ethoxy-phenyl)-1,1-dimethyl-ethylamino]-1-hydroxy-ethyl}-6-hydroxy-4H-benzo[1,4]oxazin-3-one

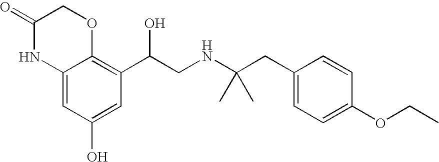 a) benzyl [2-(4-ethoxy-phenyl)-1,1-dimethyl-ethyl]-carbamate

15.0 g (50 mmol) benzyl [2-(4-hydroxy-phenyl)-1,1-dimethyl-ethyl]-carbamate are stirred with 7.5 mL (92 mmol) ethyl iodide and 21 g (150 mmol) potassium carbonate for 10 hours at 90-100° C. The reaction mixture is combined with ethyl acetate, washed twice with water and dried with sodium sulphate. After the solvents have been distilled off a yellow oil remains (15.0 g, 92%), which is further reacted directly.

b) 2-(4-ethoxy-phenyl)-1,1-dimethyl-ethylamine

A solution of 15.0 g (49 mmol) benzyl [2-(4-ethoxy-phenyl)-1,1-dimethyl-ethyl]-carbamate in 100 mL glacial acetic acid is combined with 2 g palladium on charcoal (10%) and then hydrogenated at 5 bar and 40 to 50° C. The catalyst is filtered off and the filtrate freed from solvent. The residue is dissolved in a little water, made alkaline with conc. sodium hydroxide solution and extracted with ethyl acetate. The organic phase is washed with water, dried with sodium sulphate and evaporated down. The crude product is dissolved in acetonitrile and acidified with ethereal hydrochloric acid. The solid precipitated after the addition of diethyl ether is suction filtered and dried.

Yield: 8.8 g (hydrochloride, 84%); m.p. 198-200° C.

c) 6-benzyloxy-8-{2-[2-(4-ethoxy-phenyl)-1,1-dimethyl-ethylamino]-1-hydroxy-ethyl}-4H-benzo[1,4]oxazin-3-one

2.14 g (6.0 mmol) 6-benzyloxy-8-(2-ethoxy-2-hydroxy-acetyl)-4H-benzo[1,4]oxazin-3-one and 1.0 g (5.2 mmol) 2-(4-ethoxy-phenyl)-1,1-dimethyl-ethylamine are stirred in 40 mL ethanol for one hour at 50-80° C. After cooling to ambient temperature 0.23 g (6.0 mmol) sodium borohydride are added and the mixture is stirred for a further hour. The reaction mixture is combined with 5 ml acetone, stirred for 30 minutes, acidified with glacial acetic acid and evaporated down. The residue is combined with water and ethyl acetate and made alkaline. The organic phase is separated off, washed with water, dried with sodium sulphate and freed from solvent in vacuo. The residue is dissolved again in ethyl acetate and water, combined with conc. hydrochloric acid and diluted with diethyl ether. The solid precipitated is suction filtered and washed with diethyl ether. White solid.

Yield: 2.0 g (61%, hydrochloride); m.p. 214-216° C.

d) 8-{2-[2-(4-ethoxy-phenyl)-1,1-dimethyl-ethylamino]-1-hydroxy-ethyl}-6-hydroxy-4H-benzo[1,4]oxazin-3-one

1.5 g (2.8 mmol) 6-benzyloxy-8-{2-[2-(4-ethoxy-phenyl)-1,1-dimethyl-ethylamino]-1-hydroxy-ethyl}-4H-benzo[1,4]oxazin-3-one in 80 mL methanol are hydrogenated with 250 mg palladium on charcoal (10%) as catalyst at ambient temperature and normal pressure. The catalyst is suction filtered and the filtrate is evaporated down. The residue is dissolved in 5 mL ethanol by heating, inoculated and diluted with ethyl acetate. The solid precipitated is filtered off and washed. White solid.

Yield 1.0 g (83%, hydrochloride); m.p. 232-235° C.; mass spectrometry: [M+H]⁺=401.

The (R)- and (S)-enantiomers of this embodiment may be obtained by separation of the racemate analogously to common methods known in the art.

### EXAMPLE 1.13

### 8-{2-[2-(3,5-dimethyl-phenyl)-1,1-dimethyl-ethylamino]-1-hydroxy-ethyl}-6-hydroxy-4H-benzo[1,4]oxazin-3-one

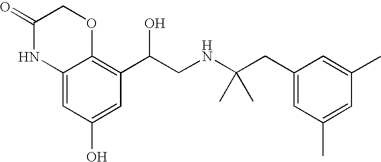 a) 1-(3,5-dimethyl-phenyl)-2-methyl-propanol-2-ol

Obtained from the reaction of ethyl (3,5-dimethyl-phenyl)-acetate with methylmagnesium bromide.

b) 2-(3,5-dimethyl-phenyl)-1,1-dimethyl-ethylamine

By reacting 6.00 g (34 mmol) 1-(3,5-dimethyl-phenyl)-2-methyl-propanol-2-ol and 2.00 g (41 mmol) sodium cyanide in a Ritter reaction, 2.40 g 2-(3,5-dimethyl-phenyl)-1,1-dimethyl-ethylformamide (35% yield) are obtained. To liberate the amine the formamide (2.40 g, 11.7 mmol) is treated with hydrochloric acid. The preparation and working up are carried out analogously to the procedure laid down for Example 1.8c). Oil.

Yield: 1.70 g (82%); mass spectroscopy: [M+H]⁺=178.

c) 6-benzyloxy-8-{2-[2-(3,5-dimethyl-phenyl)-1,1-dimethyl-ethylamino]-1-hydroxy-ethyl}-4H-benzo[1,4]oxazin-3-one

Prepared analogously to the procedure laid down for Example 1.6d) from 1.47 g (4.1 mmol) benzyloxy-8-(2-ethoxy-2-hydroxy-acetyl)-4H-benzo[1,4]oxazin-3-one and 0.65 g (3.7 mmol) 2-(3,5-dimethyl-phenyl)-1,1-dimethyl-ethylamine.

Yield: 1.1 g (51%, hydrochloride); m.p. 220-222° C.

d) 8-{2-[2-(3,5-dimethyl-phenyl)-1,1-dimethyl-ethylamino]-1-hydroxy-ethyl}-6-hydroxy-4H-benzo[1,4]oxazin-3-one

The target compound was obtained after hydrogenolysis of 0.90 g (1.71 mmol) 6-benzyloxy-8-{2-[2-(3,5-dimethyl-phenyl)-1,1-dimethyl-ethylamino]-1-hydroxy-ethyl}-4H-benzo[1,4]oxazin-3-one and recrystallisation of the crude product from isopropanol. White solid.

Yield: 0.50 g (69%, hydrochloride); m.p. 235-238° C.; mass spectroscopy: [M+H]⁺=385.

The (R)- and (S)-enantiomers of this embodiment may be obtained by separation of the racemate analogously to common methods known in the art.

### EXAMPLE 1.14

### 4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric Acid

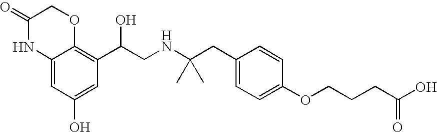 a) ethyl 4-[4-(2-amino-2-methyl-propyl)-phenoxy]-butyrate

4.5 g (15.0 mmol) benzyl [2-(4-hydroxy-phenyl)-1,1-dimethyl-ethyl]-carbamate, 2.3 mL (16.0 mmol) ethyl 4-bromo-butyrate, 2.3 g (16.6 mmol) potassium carbonate and 0.3 g (1.8 mmol) potassium iodide in 20 mL dimethylformamide are heated to 120° C. for 13 h. The reaction mixture is diluted with ethyl acetate and washed successively with water, sodium hydroxide solution and water. The organic phase is dried with sodium sulphate and evaporated down. The residue is purified by chromatography (eluant: cyclohexane/ethyl acetate=9:1). 5.0 g of a yellow oil is isolated, which is dissolved in 50 mL acetic acid and hydrogenated with 1.0 g palladium on charcoal as catalyst at 40° C. and 3 bar. The catalyst is filtered off and the filtrate is freed from solvent. The residue is dissolved in diethyl ether and combined with ethereal hydrochloric acid. The solid precipitated is suction filtered and dried. Yield: 2.9 g (66% over two steps, hydrochloride); m.p.=103-105° C.

b) ethyl 4-(4-{2-[2-(6-benzyloxy-3-oxo-3,4-dihydro-2H-benzo[1,4]oxazin-8-yl)-2-hydroxy-ethylamino]-2-methyl-propyl}-phenoxy)-butyrate

1.20 g (3.36 mmol) benzyloxy-8-(2-ethoxy-2-hydroxy-acetyl)-4H-benzo[1,4]oxazin-3-one and 0.90 g (3.22 mmol) ethyl 4-[4-(2-amino-2-methyl-propyl)-phenoxy]-butyrate are reacted in the manner described for Example 1.6d). The crude product is dissolved in 10 mL ethyl acetate and 10 mL water and combined with oxalic acid with stirring. The solution is diluted with diethyl ether and the solid precipitated is suction filtered and washed with diethyl ether. Yield: 1.20 g (54%, oxalate); m.p. 223-227° C.

c) 4-(4-{2-[2-(6-benzyloxy-3-oxo-3,4-dihydro-2H-benzo[1,4]oxazin-8-yl)-2-hydroxy-ethylamino]-2-methyl-propyl}-phenoxy)-butyric Acid

A solution of 1.00 g (1.73 mmol) ethyl 4-(4-{2-[2-(6-benzyloxy-3-oxo-3,4-dihydro-2H-benzo[1,4]oxazin-8-yl)-2-hydroxy-ethylamino]-2-methyl-propyl}-phenoxy)-butyrate in 25 mL methanol is combined with 2.5 mL 1 N sodium hydroxide solution, refluxed for 30 minutes and then neutralised with 1 N hydrochloric acid. The solution is evaporated down and the oil remaining is dissolved in 5 mL n-butanol by heating. After the addition of a crystallisation aid a solid is precipitated, which is suction filtered and washed with acetone and diethyl ether. Yield: 0.75 g (79%); m.p. 216-218° C.

d) 4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric Acid

0.70 g (1.28 mmol) 4-(4-{2-[2-(6-benzyloxy-3-oxo-3,4-dihydro-2H-benzo[1,4]oxazin-8-yl)-2-hydroxy-ethylamino]-2-methyl-propyl}-phenoxy)-butyric acid are dissolved in 25 mL methanol and 2 mL acetic acid and hydrogenated in the presence of 150 mg palladium on charcoal (10%) at ambient temperature and normal pressure. The catalyst is filtered off and the filtrate is freed from solvent. The product is obtained by crystallisation from a methanol/acetone mixture.

Yield: 0.40 g (68%); m.p. 201-204° C.; mass spectroscopy: [M+H]⁺=459.

The (R)- and (S)-enantiomers of this embodiment may be obtained by separation of the racemate analogously to common methods known in the art.

### EXAMPLE 1.15

### 8-{2-[2-(3,4-difluoro-phenyl)-1,1-dimethyl-ethylamino]-1-hydroxy-ethyl}-6-hydroxy-4H-benzo[1,4]oxazin-3-one

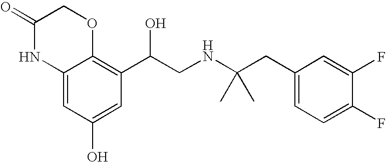 a) 1-(3,4-difluoro-phenyl)-2-methyl-propan-2-ol

A Grignard reagent is prepared from 23.0 g (111 mmol) 3,4-difluorobenzylbromide and is then reacted with 11.6 mL (158 mmol) acetone. Light yellow oil.

Yield: 9.7 g (47%); R_{f} value: 0.55 (ethyl acetate/petroleum ether=1:3).

b) N-[2-(3,4-difluoro-phenyl)-1,1-dimethyl-ethyl]-formamide

The target compound is obtained by a Ritter reaction with 4.0 g (21.5 mmol) 1-(3,4-difluoro-phenyl)-2-methyl-propan-2-ol. Light yellow oil.

Yield: 4.0 g (87%); mass spectrometry: [M+H]⁺=214.

c) 2-(3,4-difluoro-phenyl)-1,1-dimethyl-ethylamine

4.00 g (18.5 mmol) N-[2-(3,4-difluoro-phenyl)-1,1-dimethyl-ethyl]-formamide are dissolved in ethanol, combined with conc. hydrochloric acid and heated overnight at reflux temperature. The reaction solution is poured onto ice water, made alkaline with sodium hydroxide and extracted with tert-butylmethylether. The organic phases are washed with water, dried with sodium sulphate and evaporated down. Yellow oil.

Yield: 3.2 g (92%); mass spectrometry: [M+H]⁺=186.

d) 8-{2-[2-(3,4-difluoro-phenyl)-1,1-dimethyl-ethylamino]-1-hydroxy-ethyl}-6-hydroxy-4H-benzo[1,4]oxazin-3-one

357 mg (1 mmol) 6-benzyloxy-8-(2-ethoxy-2-hydroxy-acetyl)-4H-benzo[1,4]oxazin-3-one and 185 mg (1 mmol) 2-(3,4-difluoro-phenyl)-1,1-dimethyl-ethylamine are stirred for 30 minutes in 5 mL tetrahydrofuran at ambient temperature. The mixture is cooled to 0° C. and under an argon atmosphere 1.5 mL of a 2 molar solution of lithium borohydride in tetrahydrofuran is added dropwise thereto. The resulting mixture is stirred for 30 min at ambient temperature, combined with 10 mL dichloromethane and 3 mL water, stirred for a further hour and then filtered through Extrelut®. The eluate containing the ethanolamine is freed from solvent. The residue is dissolved in methanol and hydrogenated with palladium on charcoal (10%) as catalyst at 2.5 bar and ambient temperature. Then the catalyst is separated off and the crude product is purified by chromatography. White solid.

Yield: 31 mg (6%, trifluoroacetate); mass spectroscopy: [M+H]⁺=393.

The (R)- and (S)-enantiomers of this embodiment may be obtained by separation of the racemate analogously to common methods known in the art.

### EXAMPLE 1.16

### 8-{2-[2-(2-chloro-4-fluoro-phenyl)-1,1-dimethyl-ethylamino]-1-hydroxy-ethyl}-6-hydroxy-4H-benzo[1,4]oxazin-3-one

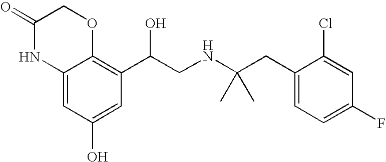 a) 1-(2-chloro-4-fluoro-phenyl)-2-methyl-propan-2-ol

Prepared from 20 g (97 mmol) methyl (2-chloro-4-fluoro-phenyl)-acetate and 98 mL of a 3 molar solution of methylmagnesium bromide analogously to the procedure laid down for Example 1.6a).

b) N-[2-(2-chloro-4-fluoro-phenyl)-1,1-dimethyl-ethyl]-formamide

7.5 g (37 mmol) 1-(2-chloro-4-fluoro-phenyl)-2-methyl-propan-2-ol were reacted and worked up according to the procedure described for Example 1.8b). The oil thus obtained was chromatographed for further purification on a short silica gel column (petroleum ether/ethyl acetate=9:1). Oil. Yield: 7.4 g (87%); mass spectrometry: [M+H]⁺=230/232.

c) 2-(2-chloro-4-fluoro-phenyl)-1,1-dimethyl-ethylamine

Reaction of 7.4 g (32 mmol) N-[2-(2-chloro-4-fluoro-phenyl)-1,1-dimethyl-ethyl]-formamide as described in the procedure laid down for Example 1. 15c). Brown oil. Yield: 5.14 g (79%); mass spectrometry: [M+H]⁺=202/204.

d) 8-{2-[2-(2-chloro-4-fluoro-phenyl)-1,1-dimethyl-ethylamino]-1-hydroxy-ethyl}-6-hydroxy-4H-benzo[1,4]oxazin-3-one

357 mg (1 mmol) 6-benzyloxy-8-(2-ethoxy-2-hydroxy-acetyl)-4H-benzo[1,4]oxazin-3-one and 202 mg (1 mmol) 2-(2-chloro-4-fluoro-phenyl)-1,1-dimethyl-ethylamine are reacted with lithium borohydride analogously to the procedure laid down for Example 1.8d). To debenzylate the ethanolamine thus obtained it is dissolved in 3 mL dichloromethane and cooled to −78° C. At this temperature 2 ml of a 1 molar solution of boron tribromide in dichloromethane is added dropwise and the mixture is slowly left to come up to ambient temperature. The reaction mixture is combined with 10 mL dichloromethane and 3 mL water and filtered through Extrelut®. The eluate is freed from solvent and the residue is purified by chromatography. White solid.

Yield: 70 mg (13%, trifluoroacetate); mass spectroscopy: [M+H]⁺=409/11.

The (R)- and (S)-enantiomers of this embodiment may be obtained by separation of the racemate analogously to common methods known in the art.

### EXAMPLE 1.17

### 8-{2-[2-(4-chloro-phenyl)-1,1-dimethyl-ethylamino]-1-hydroxy-ethyl}-6-hydroxy-4H-benzo[1,4]oxazin-3-one

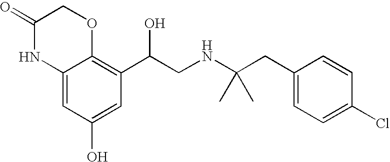

A solution of 300 mg (0.91 mmol) 6-benzyloxy-8-(2,2-dihydroxy-acetyl)-4H-benzo[1,4]oxazin-3-one and 200 mg (1.09 mmol) 2-(4-chloro-phenyl)-1,1-dimethyl-ethylamine in 3 mL ethanol was combined with molecular sieve and stirred for 90 minutes at 80° C. The mixture was allowed to cool to ambient temperature, 35 mg (0.91 mmol) of sodium borohydride were added and the resulting mixture was stirred for 1 hour. Then the reaction mixture was combined with sodium hydrogen carbonate solution and extracted with ethyl acetate. The combined organic phases were freed from solvent and the residue was chromatographed (eluant: hexane/ethyl acetate/methanol), thus yielding 305 mg ethanolamine. This was dissolved in 3 mL dichloromethane and cooled to −78° C. under an argon atmosphere. 3 mL of a 1 molar solution of boron tribromide in dichloromethane were added dropwise and the mixture was stirred for one hour at −78° C. and 20 minutes at ambient temperature. Then at −78° C. 3 mL conc. ammonia solution were added dropwise and the mixture was stirred for 5 minutes. The reaction mixture was combined with ammonium chloride solution and extracted with ethyl acetate. The combined organic phases were evaporated down and the residue was chromatographed for further purification (silica gel; eluant:dichloromethane/methanol+1% ammonia). Beige solid: 93 mg (26%); mass spectrometry: [M+H]⁺=391.

The (R)- and (S)-enantiomers of this embodiment may be obtained by separation of the racemate analogously to common methods known in the art.

### EXAMPLE 1.18

### 8-{2-[2-(4-bromo-phenyl)-1,1-dimethyl-ethylamino]-1-hydroxy-ethyl}-6-hydroxy-4H-benzo[1,4]oxazin-3-one

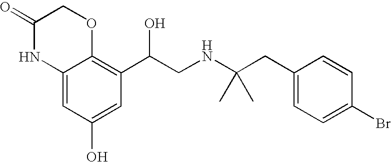

The preparation and debenzylation of the ethanolamine were carried out as described for Example 1.17 from 300 mg (0.91 mmol) 6-benzyloxy-8-(2,2-dihydroxy-acetyl)-4H-benzo[1,4]oxazin-3-one and 250 mg (1.09) mmol) 2-(4-bromo-phenyl)-1,1-dimethyl-ethylamine. Beige solid. Yield: 54 mg (14%); mass spectrometry: [M+H]⁺=435, 437.

The (R)- and (S)-enantiomers of this embodiment may be obtained by separation of the racemate analogously to common methods known in the art.

### Example 1.19

### 8-{2-[2-(4-fluoro-phenyl)-1,1-dimethyl-ethylamino]-1-hydroxy-ethyl}-6-hydroxy-4H-benzo[1,4]oxazin-3-one

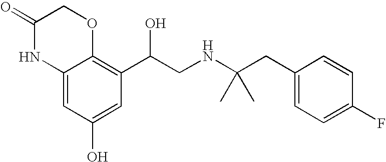

300 mg (0.91 mmol) 6-benzyloxy-8-(2,2-dihydroxy-acetyl)-4H-benzo[1,4]oxazin-3-one and 183 mg (1.09 mmol) 2-(4-fluoro-phenyl)-1,1-dimethyl-ethylamine were dissolved in 3 ml ethanol. Molecular sieve was added and the mixture was heated to 80° C. for 30 minutes. After cooling to ambient temperature 35 mg (0.91 mmol) sodium borohydride were added. The mixture was stirred for 1 hour at ambient temperature rühren, then sodium hydrogen carbonate solution was added to the reaction mixture and this was extracted with ethyl acetate. The organic phases were evaporated down and the residue was chromatographed (eluant:hexane/ethyl acetate/methanol). The ethanolamine thus obtained (223 mg) was dissolved in methanol in order to cleave the benzyl protecting group and hydrogenated with 150 mg palladium hydroxide as catalyst at ambient temperature and normal pressure. The catalyst was separated off by filtration through Celite®, the filtrate was freed from solvent and the residue was chromatographed (silica gel; eluant:dichloromethane/methanol). Beige solid.

Yield: 76 mg (22%); mass spectrometry: [M+H]⁺=375.

The (R)- and (S)-enantiomers of this embodiment may be obtained by separation of the racemate analogously to common methods known in the art.

The following compounds of formula 1 according to the invention may also be obtained analogously to the synthesis examples described hereinbefore:

### EXAMPLE 1.20

### 8-{2-[2-(4-fluoro-3-methoxy-phenyl)-1,1-dimethyl-ethylamino]-1-hydroxy-ethyl}-6-hydroxy-4H-benzo[1,4]oxazin-3-one;

### EXAMPLE 1.21

### 8-{2-[2-(4-fluoro-2,6-dimethyl-phenyl)-1,1-dimethyl-ethylamino]-1-hydroxy-ethyl}-6-hydroxy-4H-benzo[1,4]oxazin-3-one;

### EXAMPLE 1.22

### 8-{2-[2-(4-chloro-2-methyl-phenyl)-1,1-dimethyl-ethylamino]-1-hydroxy-ethyl}-6-hydroxy-4H-benzo[1,4]oxazin-3-one;

### EXAMPLE 1.23

### 8-{2-[2-(4-chloro-3-fluoro-phenyl)-1,1-dimethyl-ethylamino]-1-hydroxy-ethyl}-6-hydroxy-4H-benzo[1,4]oxazin-3-one;

### EXAMPLE 1.24

### 8-{2-[2-(4-chloro-2-fluoro-phenyl)-1,1-dimethyl-ethylamino]-1-hydroxy-ethyl}-6-hydroxy-4H-benzo[1,4]oxazin-3-one;

### EXAMPLE 1.25

### 8-{2-[2-(3-chloro-4-fluoro-phenyl)-1,1-dimethyl-ethylamino]-1-hydroxy-ethyl}-6-hydroxy-4H-benzo[1,4]oxazin-3-one;

### EXAMPLE 1.26

### 8-{2-[2-(2,6-difluoro-4-methoxy-phenyl)-1,1-dimethyl-ethylamino]-1-hydroxy-ethyl}-6-hydroxy-4H-benzo[1,4]oxazin-3-one;

### EXAMPLE 1.27

### 8-{2-[2-(2.5-difluoro-4-methoxy-phenyl)-1,1-dimethyl-ethylamino]-1-hydroxy-ethyl}-6-hydroxy-4H-benzo[1,4]oxazin-3-one;

### EXAMPLE 1.28

### 8-{2-[2-(4-fluoro-3,5-dimethyl-phenyl)-1,1-dimethyl-ethylamino]-1-hydroxy-ethyl}-6-hydroxy-4H-benzo[1,4]oxazin-3-one;

### EXAMPLE 1.29

### 8-{2-[2-(3,5-dichloro-phenyl)-1,1-dimethyl-ethylamino]-1-hydroxy-ethyl}-6-hydroxy-4H-benzo[1,4]oxazin-3-one;

### EXAMPLE 1.30

### 8-{2-[2-(4-chloro-3-methyl-phenyl)-1,1-dimethyl-ethylamino]-1-hydroxy-ethyl}-6-hydroxy-4H-benzo[1,4]oxazin-3-one;

### EXAMPLE 1.31

### 8-{2-[2-(3,4.5-trifluoro-phenyl)-1,1-dimethyl-ethylamino]-1-hydroxy-ethyl}-6-hydroxy-4H-benzo[1,4]oxazin-3-one;

### EXAMPLE 1.32

### 8-{2-[2-(3-methyl-phenyl)-1,1-dimethyl-ethylamino]-1-hydroxy-ethyl}-6-hydroxy-4H-benzo[1,4]oxazin-3-one;

### EXAMPLE 1.33

8-{2-[2-(3,4-dichloro-phenyl)-1,1-dimethyl-ethylamino]-1-hydroxy-ethyl}-6-hydroxy-4H-benzo[1,4]oxazin-3-one.

1) A pharmaceutical composition comprising one or more compounds of formula 1 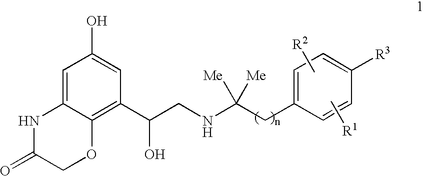
wherein
n denotes 1 or 2;
R¹ denotes hydrogen, halogen, C₁-C₄-alkyl or —O—C₁-C₄-alkyl;
R² denotes hydrogen, halogen, C₁-C₄-alkyl or —O—C₁-C₄-alkyl;
R³ denotes C₁-C₄-alkyl, OH, halogen, —O—C₁-C₄-alkyl, —O—C₁-C₄-alkylene-COOH, —O—C₁-C₄-alkylene-CO—O—C₁-C₄-alkyl, and
at least one other active substance 2.

2) The pharmaceutical composition according to claim 1, wherein the additional active substance 2 is one or more compounds which are selected from the group consisting of anticholinergics (2a), PDE IV inhibitors (2b), steroids (2c), LTD4-antagonists (2d) and EGFR-inhibitors (2e).

3) The pharmaceutical composition according to claim 1 comprising one or more compounds of formula 1, wherein
n denotes 1 or 2;
R¹ denotes hydrogen, fluorine, chlorine, methyl or methoxy;
R² denotes hydrogen, fluorine, chlorine, methyl or methoxy;
R³ denotes C₁-C₄-alkyl, OH, fluorine, chlorine, bromine, —O—C₁-C₄-alkyl, —O—C₁-C₄-alkylene-COOH, —O—C₁-C₄-alkylene-CO—O—C₁-C₄-alkyl.

4) The pharmaceutical composition according to claim 1 comprising one or more compounds of formula 1, wherein
n denotes 1;
R¹ denotes hydrogen or C₁-C₄-alkyl;
R² denotes hydrogen or C₁-C₄-alkyl;
R³ denotes C₁-C₄-alkyl, OH, —O—C₁-C₄-alkyl, —O—C₁-C₄-alkylene-COOH or —O—C₁-C₄-alkylene-CO—O—C₁-C₄-alkyl.

5) The pharmaceutical composition according to claim 1, wherein the one or more compounds of formula 1 are in the form of the individual optical isomers, mixtures of the individual enantiomers or racemates.

6) The pharmaceutical composition according to claim 1, wherein the one or more compounds of formula 1 are in the form of the acid addition salts with pharmacologically acceptable acids as well as optionally in the form of the solvates and/or hydrates.

7) The pharmaceutical composition according to claim 1, wherein the additional active substance 2 is an anticholinergic (2a).

8) The pharmaceutical composition according to claim 7, wherein the anticholinergic (2a) is selected from the group consisting of tiotropium salts (2a.1), oxitropium salts (2a.2), flutropium salts (2a.3), ipratropium salts (2a.4), glycopyrronium salts (2a.5), and trospium salts (2a.6).

9) The pharmaceutical composition according to claim 7, wherein the anticholinergic is of the formula 2a.7 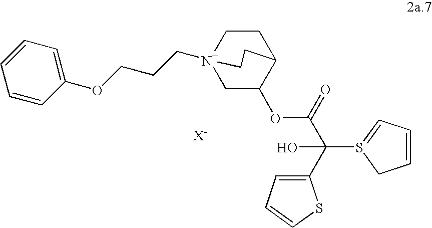
wherein
X⁻ denotes an anion with a single negative charge, preferably an anion selected from among the fluoride, chloride, bromide, iodide, sulphate, phosphate, methanesulphonate, nitrate, maleate, acetat, citrate, fumarate, tartrate, oxalate, succinate, benzoate and p-toluenesulphonate,
optionally in the form of the racemates, enantiomers or hydrates thereof.

10) The pharmaceutical composition according to claim 7, wherein the anticholinergic is of the formula 2a.8 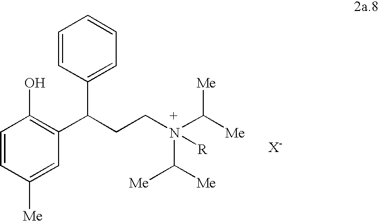
wherein R denotes either methyl (2a.8.1) or ethyl (2a.8.2) and
wherein
X⁻ denotes an anion with a single negative charge, preferably an anion selected from among the fluoride, chloride, bromide, iodide, sulphate, phosphate, methanesulphonate, nitrate, maleate, acetat, citrate, fumarate, tartrate, oxalate, succinate, benzoate and p-toluenesulphonate,
optionally in the form of the racemates, enantiomers or hydrates thereof.

11) The pharmaceutical composition according to claim 7, wherein the anticholinergic is of the formula 2a.9 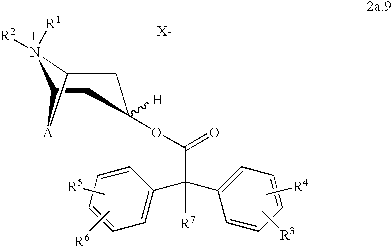
wherein
A denotes a double-bonded group selected from the groups 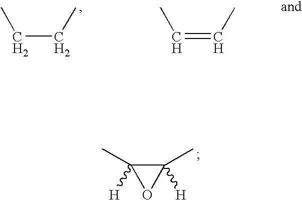
X⁻ denotes an anion with a single negative charge, preferably an anion selected from among the fluoride, chloride, bromide, iodide, sulphate, phosphate, methanesulphonate, nitrate, maleate, acetat, citrate, fumarate, tartrate, oxalate, succinate, benzoate and p-toluenesulphonate;
R¹ and R² which may be identical or different denote a group selected from methyl, ethyl, n-propyl and iso-propyl, which may optionally be substituted by hydroxy or fluorine, preferably unsubstituted methyl;
R³, R⁴, R⁵ and R⁶, which may be identical or different, denote hydrogen, methyl, ethyl, methyloxy, ethyloxy, hydroxy, fluorine, chlorine, bromine, CN, CF₃ or NO₂;
R⁷ denotes hydrogen, methyl, ethyl, methyloxy, ethyloxy, —CH₂—F, —CH₂—CH₂—F, —O—CH₂—F, —O—CH₂—CH₂—F, —CH₂—OH, —CH₂—CH₂—OH, CF₃, —CH₂—OMe, —CH₂—CH₂—OMe, —CH₂—OEt, —CH₂—CH₂—OEt, —O—COMe, —O—COEt, —O—COCF₃, —O—COCF₃, fluorine, chlorine or bromine,
optionally in the form of the racemates, enantiomers or hydrates thereof.

12) The pharmaceutical composition according to claim 7, wherein the anticholinergic is of the formula 2a.10 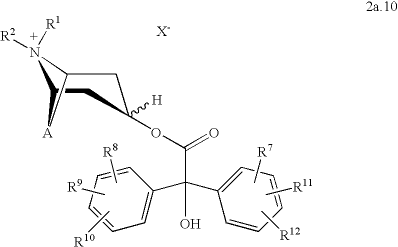
wherein
A denotes a double-bonded group selected from the groups 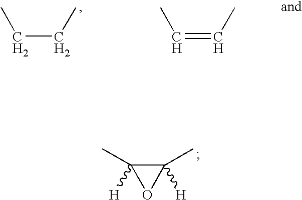
X⁻ denotes an anion with a single negative charge, preferably an anion selected from among the fluoride, chloride, bromide, iodide, sulphate, phosphate, methanesulphonate, nitrate, maleate, acetat, citrate, fumarate, tartrate, oxalate, succinate, benzoate and p-toluenesulphonate;
R¹ and R² which may be identical or different denote a group selected from methyl, ethyl, n-propyl and iso-propyl, which may optionally be substituted by hydroxy or fluorine, preferably unsubstituted methyl; and
R⁷, R⁸, R⁹, R¹⁰, R¹¹ and R¹² may be identical or different, denote hydrogen, methyl, ethyl, methyloxy, ethyloxy, hydroxy, fluorine, chlorine, bromine, CN, CF₃ or NO₂, while at least one of the groups R⁷, R⁸, R⁹, R¹⁰, R¹¹ and R¹² may not be hydrogen,
optionally in the form of the racemates, enantiomers or hydrates thereof.

13) The pharmaceutical composition according to claim 7, wherein the anticholinergic is of the formula 2a.11 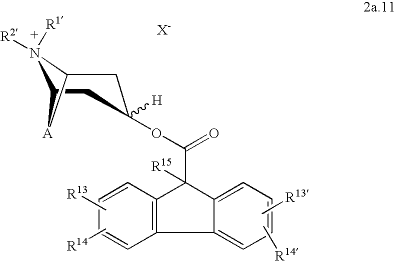
wherein
A denotes a double-bonded group selected from the groups 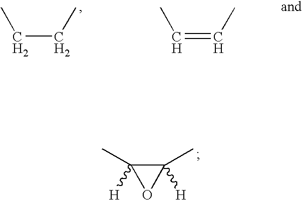
X⁻ denotes an anion with a single negative charge, preferably an anion selected from among the fluoride, chloride, bromide, iodide, sulphate, phosphate, methanesulphonate, nitrate, maleate, acetat, citrate, fumarate, tartrate, oxalate, succinate, benzoate and p-toluenesulphonate;
R¹⁵ denotes hydrogen, hydroxy, methyl, ethyl, —CF₃, CHF₂ or fluorine;
R^{1′} and R^{2′} which may be identical or different, denote C₁-C₅-alkyl, which may optionally be substituted by C₃-C₆-cycloalkyl, hydroxy or halogen, or
R^{1′} and R^{2′} together denote a —C₃-C₅-alkylene bridge;
R¹³, R¹⁴, R^{13′} and R^{14′} which may be identical or different, denote hydrogen, —C₁-C₄-alkyl, —C₁-C₄-alkyloxy, hydroxy, —CF₃, —CHF₂, CN, NO₂ or halogen,
optionally in the form of the racemates, enantiomers or hydrates thereof.

14) The pharmaceutical composition according to claim 7, wherein the anticholinergic is of the formula 2a.12 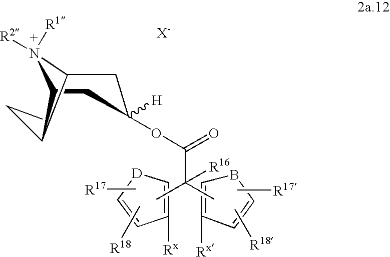
wherein
X⁻ denotes an anion with a single negative charge, preferably an anion selected from among the fluoride, chloride, bromide, iodide, sulphate, phosphate, methanesulphonate, nitrate, maleate, acetat, citrate, fumarate, tartrate, oxalate, succinate, benzoate and p-toluenesulphonate;
D and B which may be identical or different, preferably identical, denote O, S, NH, CH₂, CH═CH or N(C₁-C₄-alkyl);
R^{1 6} denotes hydrogen, hydroxy, —C₁-C₄-alkyl, —C₁-C₄-alkyloxy, —C₁-C₄-alkylene-halogen, —O—C₁-C₄-alkylene-halogen, —C₁-C₄-alkylene-OH, —CF₃, CHF₂, —C₁-C₄-alkylene-C₁-C₄-alkyloxy, —O—COC₁-C₄-alkyl, —O—COC₁-C₄-alkylene-halogen, —C₁-C₄-alkylene-C₃-C₆-cycloalkyl, —O—COCF₃ or halogen;
R^{1″} and R^{2″} which may be identical or different, denote —C₁-C₅-alkyl, which may optionally be substituted by —C₃-C₆-cycloalkyl, hydroxy or halogen, or
R^{1″} and R^{2″} together denote a —C₃-C₅-alkylene bridge;
R¹⁷, R¹⁸, R^{17′} and R^{18′}, which may be identical or different, denote hydrogen, —C₁-C₄-alkyl, —C₁-C₄-alkyloxy, hydroxy, —CF₃, —CHF₂, CN, NO₂ or halogen;
R^{X} and R^{X′} which may be identical or different, denote hydrogen, —C₁-C₄-alkyl, —C₁-C₄-alkyloxy, hydroxy, —CF₃, —CHF₂, CN, NO₂ or halogen, or
R^{X} and R^{X′} together denote a single bond or one of the double-bonded groups O, S, NH, CH₂, CH₂—CH₂, N(C₁-C₄-alkyl), CH(C₁-C₄-alkyl) and —C(C₁-C₄-alkyl)₂,
optionally in the form of the racemates, enantiomers or hydrates thereof.

15) The pharmaceutical composition according to claim 7, wherein the anticholinergic is of the formula 2a.13 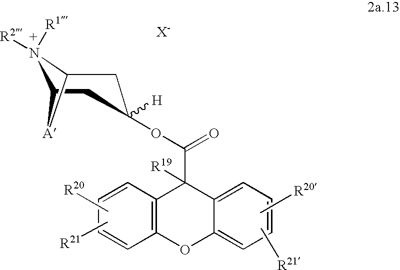
wherein
X⁻ denotes an anion with a single negative charge, preferably an anion selected from among the fluoride, chloride, bromide, iodide, sulphate, phosphate, methanesulphonate, nitrate, maleate, acetat, citrate, fumarate, tartrate, oxalate, succinate, benzoate and p-toluenesulphonate;
A′ denotes a double-bonded group selected from 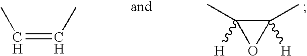
R¹⁹ denotes hydroxy, methyl, hydroxymethyl, ethyl, —CF₃, CHF₂ or fluorine;
R^{1′″} and R^{2′″} which may be identical or different, denote C₁-C₅-alkyl, which may optionally be substituted by C₃-C₆-cycloalkyl, hydroxy or halogen, or
R^{′″} and R^{2′″} together denote a —C₃-C₅-alkylene bridge;
R²⁰, R²¹, R^{20′} and R^{21′} which may be identical or different, denote hydrogen, —C₁-C₄-alkyl, —C ₁-C₄-alkyloxy, hydroxy, —CF₃, —CHF₂, CN, NO₂ or halogen,
optionally in the form of the racemates, enantiomers or hydrates thereof.

16) The pharmaceutical composition according to claim 1, wherein the additional active substance 2 a PDE IV-inhibitor (2b).

17) The pharmaceutical composition according to claim 16, wherein the PDE IV-inhibitor 2b is selected from the group consisting of enprofyllin, theophyllin, roflumilast, ariflo (cilomilast), CP-325.366, BY343, D-4396 (Sch-351591), AWD-12-281 (GW-842470), N-(3,5-dichloro-1-oxo-pyridin-4-yl)-4-difluoromethoxy-3-cyclopropylmethoxybenzamide, NCS-613, pumafentine, (−)p-[(4aR*,10bS*)-9-ethoxy-1,2,3,4,4a.10b-hexahydro-8-methoxy-2-methylbenzo[s][1,6]naphthyridin-6-yl]-N,N-diisopropylbenzamide, (R)-(+)-1-(4-bromobenzyl)-4-[(3-cyclopentyloxy)-4-methoxyphenyl]-2-pyrrolidone, 3-(cyclopentyloxy-4-methoxyphenyl)-1-(4-N′-[N-2-cyano-S-methyl-isothioureido]benzyl)-2-pyrrolidone, cis[4-cyano-4-(3-cyclopentyloxy-4-methoxyphenyl)cyclohexane-1-carboxylic acid], 2-carbomethoxy-4-cyano-4-(3-cyclopropylmethoxy-4-difluoromethoxyphenyl)cyclohexan-1-one, cis[4-cyano-4-(3-cyclopropylmethoxy-4-difluoromethoxyphenyl)cyclohexan-1-ol], (R)-(+)-ethyl[4-(3-cyclopentyloxy-4-methoxyphenyl)pyrrolidin-2-ylidene]acetate, (S)-(−)-ethyl[4-(3-cyclopentyloxy-4-methoxyphenyl)pyrrolidin-2-ylidene]acetate, CDP840, Bay-198004, D-4418, PD-168787, T-440, T-2585, arofyllin, atizoram, V-11294A, Cl-1018, CDC-801, CDC-3052, D-22888, YM-58997, Z-15370, 9-cyclopentyl-5,6-dihydro-7-ethyl-3-(2-thienyl)-9H-pyrazolo[3,4-c]-1,2,4-triazolo[4.3-a]pyridine and 9-cyclopentyl-5,6-dihydro-7-ethyl-3-(tert-butyl)-9H-pyrazolo[3,4-c]-1,2,4-triazolo[4,3-a]pyridine, optionally in the form of the racemates, enantiomers or diastereomers thereof and optionally in the form of the pharmacologically acceptable acid addition salts, solvates and/or hydrates thereof.

18) The pharmaceutical composition according to claim 1, wherein the additional active substance 2 is a steroid (2c).

19) The pharmaceutical composition according to claim 18, wherein the steroid 2c is selected from the group consisting of prednisolone (2c.1), prednisone (2c.2), butixocortpropionate (2c.3), RPR-106541 (2c.4), flunisolide (2c.5), beclomethasone (2c.6), triamcinolone (2c.7), budesonide (2c.8), fluticasone (2c.9 , mometasone (2c.10), ciclesonide (2c.11), rofleponide (2c.12), ST-126 (2c.13), dexamethasone (2c.14), (S)-fluoromethyl 6α,9α-difluoro-17α-[(2-furanylcarbonyl)oxy]-11β-hydroxy-16α-methyl-3-oxo-androsta-1,4-diene-17β-carbothionate (2c.15), (S)-(2-oxo-tetrahydro-furan-3S-yl) 6α,9α-difluoro-11β-hydroxy-16α-methyl-3-oxo-17α-propionyloxy-androsta-1,4-diene-17β-carbothionate (2c.16) and etiprednol-dichloroacetate (2c.17), optionally in the form of the racemates, enantiomers or diastereomers thereof and optionally in the form of the salts and derivatives thereof, the solvates and/or hydrates thereof.

20) The pharmaceutical composition according to claim 1, wherein the additional active substance 2 is a LTD4-antagonist (2d).

21) The pharmaceutical composition according to claim 20, wherein the LTD4 antagonist 2d is selected from the group consisting of montelukast (2d.1), 1-(((R)-(3-(2-(6,7-difluoro-2-quinolinyl)ethenyl)phenyl)-3-(2-(2-hydroxy-2-propyl)phenyl)thio)-methylcyclopropane-acetic acid (2d.2), 1-(((1(R)-3(3-(2-(2.3-dichlorothieno[3,2-b]pyridin-5-yI)-(E)-ethenyl)phenyl)-3-(2-(1-hydroxy-1-methylethyl)phenyl)propyl)thio)methyl)-cyclopropaneacetic acid (2d.3), pranlukast (2d.4), zafirlukast (2d.5), [2-[[2-(4-tert-butyl-2-thiazolyl)-5-benzofuranyl]oxymethyl]phenyl]acetic acid (2d.6), MCC-847 (ZD-3523) (2d.7), MN-001 (2d.8), MEN-91507 (LM-1507) (2.9), VUF-5078 (2d.10), VUF-K-8707 (2d.11) and L-733321 (2d.12), optionally in the form of the racemates, enantiomers or diastereomers thereof, optionally in the form of the pharmacologically acceptable acid addition salts thereof as well as optionally in the form of the salts and derivatives thereof, the solvates and/or hydrates thereof.

22) The pharmaceutical composition according to claim 1, wherein the additional active substance 2 is an EGFR-inhibitor (2e).

23) The pharmaceutical composition according to claim 22, wherein EGFR inhibitors 2e are selected from the group consisting of 4-[(3-chloro-4-fluorophenyl)amino]-6-{[4-(morpholin-4-yl)-1-oxo-2-buten-1-yl]amino}-7-cyclopropylmethoxy-quinazoline, 4-[(3-chloro-4-fluorophenyl)amino]-6-{[4-(N,N-diethylamino)-1-oxo-2-buten-1-yl]amino}-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopentyloxy-quinazoline, 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline, 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline, 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N-cyclopropyl-N-methyl-amino)-1-oxo-2-buten-1-yl]amino}-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline, 4-[(3-ethynyl-phenyl)amino]-6,7-bis-(2-methoxy-ethoxy)-quinazoline, 4-[(3-chloro-4-fluorophenyl)amino]-7-[3-(morpholin-4-yl)-propyloxy]-6-[(vinylcarbonyl)amino]-quinazoline, 4-[(R)-(1-phenyl-ethyl)amino]-6-(4-hydroxy-phenyl)-7H-pyrrolo[2.3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-{[3-chloro-4-(3-fluoro-benzyloxy)-phenyl]amino}-6-(5-{[(2-methanesulphonyl-ethyl)amino]methyl}-furan-2-yl)quinazoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline, 4-[(3-ethynyl-phenyl)amino]-6-{[4-(5 .5-dimethyl-2-oxo-morpholin-4-yl)-1-oxo-2-buten-1-yl]amino}-quinazoline, 4-[(3-chloro-4-fluoro-phenyl)amino]-6-[2-(2,2-dimethyl-6-oxo-morpholin-4-yl)-ethoxy]-7-methoxy-quinazoline, 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline, 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline, 4-[(3-chloro-4-fluoro-phenyl)amino]-6-{2-[4-(2-oxo-morpholin-4-yl)-piperidin-1-yl]-ethoxy}-7-methoxy-quinazoline, 4-[(3-chloro-4-fluoro-phenyl)amino]-6-[1-(tert.-butyloxycarbonyl)-piperidin-4-yloxy]-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-(1-methyl-piperidin-4-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(methoxymethyl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-((S)-tetrahydrofuran-3-yloxy)-7-hydroxy-quinazoline, 4-[(3-chloro-4-fluoro-phenyl)amino]-6-(tetrahydropyran-4-yloxy)-7-(2-methoxy-ethoxy)-quinazoline, 4-[(3-chloro-4-fluoro-phenyl)amino]-6-{trans-4-[(dimethylamino)sulphonylamino]-cyclohexan-1-yloxy}-7-methoxy-quinazoline, 4-[(3-chloro-4-fluoro-phenyl)amino]-6-{trans4-[(morpholin-4-yl)carbonylamino]-cyclohexan-1-yloxy}-7-methoxy-quinazoline, 4-[(3-chloro-4-fluoro-phenyl)amino]-6-{trans-4-[(morpholin-4-yl)sulphonylamino]-cyclohexan-1-yloxy}-7-methoxy-quinazoline, 4-[(3-chloro-4-fluoro-phenyl)amino]-6-(tetrahydropyran-4-yloxy)-7-(2-acetylamino-ethoxy)-quinazoline, 4-[(3-chloro-4-fluoro-phenyl)amino]-6-(tetrahydropyran-4-yloxy)-7-(2-methanesulphonylamino-ethoxy)-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(1-aminocarbonylmethyl-piperidin-4-yloxy)-7-methoxy-quinazoline, 4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline, 4-[(3-chloro-4-fluoro-phenyl)amino]-6-(trans-4-ethanesulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-ethoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-chloro-4-fluoro-phenyl)amino]-6-(cis-4-acetylamino-cyclohexan-1-yloxy)-7-methoxy-quinazoline, 4-[(3-ethynyl-phenyl)amino]-6-[1-(tert.-butyloxycarbonyl)-piperidin-4-yloxy]-7-methoxy-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-chloro-4-fluoro-phenyl)amino]-6-{1-[(morpholin-4-yl)carbonyl]-piperidin-4-yloxy}-7-(2-methoxy-ethoxy)-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-chloro-4-fluoro-phenyl)amino]-6-(1-isopropyloxycarbonyl-piperidin-4-yloxy)-7-methoxy-quinazoline, 4-[(3-chloro-4-fluoro-phenyl)amino]-6-(cis-4-methylamino-cyclohexan-1-yloxy)-7-methoxy-quinazoline, 4-[(3-chloro-4-fluoro-phenyl)amino]-6-{cis-4-[N-(2-methoxy-acetyl)-N-methyl-amino]-cyclohexan-1-yloxy}-7-methoxy-quinazoline, 4-[(3-ethynyl-phenyl)amino]-6-(piperidin-4-yloxy)-7-methoxy-quinazoline, 4-[(3-ethynyl-phenyl)amino]-6-[1-(2-methoxy-acetyl)-piperidin-4-yloxy]-7-methoxy-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(cis-2,6-dimethyl-morpholin-4-yl)carbonyl]-piperidin-4-yloxy}-7-methoxy-quinazoline, 4-[(3-chloro-4-fluoro-phenyl)amino]-6-{1-[(2-methyl-morpholin-4-yl)carbonyl]-piperidin-4-yloxy}-7-methoxy-quinazoline, 4-[(3-chloro-4-fluoro-phenyl)amino]-6-{1-[(S,S)-(2-oxa-5-aza-bicyclo[2.2.1]hept-5-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-{1-[(2-methoxyethyl)carbonyl]-piperidin-4-yloxy}-7-methoxy-quinazoline, 4-[(3-chloro-4-fluoro-phenyl)amino]-6-{1-[(3-methoxypropyl-amino)-carbonyl]-piperidin-4-yloxy}-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-(methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, Cetuximab, Trastuzumab, ABX-EGF and Mab ICR-62, optionally in the form of the racemates, enantiomers or diastereomers thereof, optionally in the form of the pharmacologically acceptable acid addition salts thereof, the solvates and/or hydrates thereof.

24) The pharmaceutical composition according to claim 1, comprising therapeutically effective amounts of one or more of compounds of formula 1, therapeutically effective amounts of an anticholinergic (2a) and therapeutically effective amounts of a PDEIV-inhibitor (2b), and optionally pharmaceutically acceptable carriers or excipients thereof.

25) The pharmaceutical composition according to claim 1, comprising therapeutically effective amounts of compounds of formula 1, therapeutically effective amounts of an anticholinergic (2a), and therapeutically effective amounts of a steroid (2c), and optionally pharmaceutically acceptable carriers or excipients thereof.

26) The pharmaceutical composition according to claim 1, comprising therapeutically effective amounts of compounds of formula 1, therapeutically effective amounts of an anticholinergic (2a) and therapeutically effective amounts of an LTD4-antagonist (2d), and optionally pharmaceutically acceptable carriers or excipients thereof.

27) The pharmaceutical composition according to claim 1, comprising therapeutically effective amounts of compounds of formula 1, therapeutically effective amounts of an anticholinergic (2a) and therapeutically effective amounts of an EGFR-inhibitor (2e), and optionally pharmaceutically acceptable carriers or excipients thereof.

28) The pharmaceutical composition according to claim 1, comprising therapeutically effective amounts of compounds of formula 1, therapeutically effective amounts of a PDEIV-inhibitor (2b), and therapeutically effective amounts of a steroid (2c), and optionally pharmaceutically acceptable carriers or excipients thereof.

29) The pharmaceutical composition according to claim 1, comprising therapeutically effective amounts of compounds of formula 1, therapeutically effective amounts of a PDEIV-inhibitor (2b), and therapeutically effective amounts of an LTD4-antagonist (2d), and optionall pharmaceutically acceptable carriers or excipients thereof.

30) The pharmaceutical composition according to claim 1, comprising therapeutically effective amounts of compounds of formula 1, therapeutically effective amounts of a PDEIV-inhibitor (2b) and therapeutically effective amounts of an EGFR-inhibitor (2e), and optionally pharmaceutically acceptable carriers or excipients thereof.

31) The pharmaceuticals composition according to claim 1, comprising therapeutically effective amounts of 1, therapeutically effective amounts of a steroid (2c), and therapeutic amounts of an LTD4-antagonist (2d) and optionally a pharmaceutically acceptable carrier.

32) The pharmaceutical composition according to claim 1, comprising therapeutically effective amounts of compounds of formula 1, therapeutically effective amounts of a steroid (2c), and therapeutically effective amounts of an EGFR-inhibitor (2e), and optionally pharmaceutically acceptable carriers or excipients thereof.

33) The pharmaceutical composition according to claim 1, comprising therapeutically effective amounts of compounds of formula a, therapeutically effective amounts of an LTD4-antagonist (2d), and therapeutically effective amounts of an EGFR-inhibitor (2e), and optionally pharmaceutically acceptable carriers or excipients thereof.

34) The pharmaceutical composition according to claim 1, further comprising pharmaceutically acceptable carriers or excipients thereof.

35) The pharmaceutical composition according to claim 1, wherein the composition does not comprise any pharmaceutically acceptable carriers or excipients thereof.

36) The pharmaceutical composition according to claim 1, which is in the form of a formulation suitable for inhalation.

37) The pharmaceutical composition according to claim 36, wherein the inhalation formulation is selected from the group consisting of inhalable powders, propellant-driven metered-dose aerosols and propellant-free inhalable solutions or suspensions.

38) The pharmaceutical composition according to claim 37, wherein the inhalable powder comprises one or more compounds of formula 1 and active substance 2 in admixture with suitable physiologically acceptable excipients selected from the group consisting of monosaccharides, disaccharides, oligo- and polysaccharides, polyalcohols, salts, or mixtures thereof.

39) The pharmaceutical composistion according to claim 37, wherein the propellant-drive inhalable aerosol comprises one or more compounds of formula 1 and active substance 2 in dispersed form.

40) The pharmaceutical composition according to claim 39, wherein the inhalable aerosol comprises as the propellant gas hydrocarbons selected from n-propane, n-butane or isobutane or halohydrocarbons selected from chlorinated and/or fluorinated derivatives of methane, ethane, propane, butane, cyclopropane or cyclobutane.

41) The pharmaceutical composition according to claim 40, wherein the propellant gas is selected from TG11, TG12, TG134a, TG227 or mixtures thereof.

42) The pharmaceutical composition according to claim 37, wherein the propellant-free inhalable solution or suspension comprises solvent water, ethanol or a mixture of water and ethanol.

43) A method of treating inflammatory and obstructive respiratory complaints, inhibiting premature labour in midwifery (tocolysis), sinus rhythm in the heart in atrioventricular block, bradycardic heart rhythm disorders (antiarrhythmic), circulatory shock (vasodilatation and increasing the heart volume) and skin irritations and inflammation comprising administering to a patient in need thereof a therapeutically effective amount of a composition according to claim 1.

44) The method according to claim 43, wherein the respiratory complaints are selected from the group consisting of obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

45) The method according to claim 44, wherein the obstructive pulmonary diseases are selected from among bronchial asthma, paediatric asthma, severe asthma, acute asthma attacks, chronic bronchitis and COPD (chronic obstructive pulmonary disease.

46) The method according to claim 44, wherein the pulmonary emphysema which has its origins in COPD or α1-proteinase inhibitor deficiency.

47) The method according to claim 44, wherein the restrictive pulmonary diseases are selected from among allergic alveolitis, restrictive pulmonary diseases triggered by work-related noxious substances, such as asbestosis or silicosis, and restriction caused by lung tumours selected from lymphangiosis carcinomatosa, bronchoalveolar carcinoma and lymphomas.

48) The method according to claim 44, wherein the interstitial pulmonary diseases are selected from among pneumonia caused by infections selected from viruses, bacteria, fungi, protozoa, helminths or other pathogens, pneumonitis caused by various factors selected from aspiration and left heart insufficiency, radiation-induced pneumonitis or fibrosis, collagenoses selected from lupus erythematodes, systemic sclerodermy or sarcoidosis, granulomatoses selected from Boeck's disease, idiopathic interstitial pneumonia or idiopathic pulmonary fibrosis (IPF).

49) The method according to claim 44, wherein the respiratory complaint is cystic fibrosis or mucoviscidosis.

50) The method according to claim 44, wherein the respiratory complaint is bronchitis caused by bacterial or viral infection, allergic bronchitis or toxic bronchitis.

51) The method according to claim 44, wherein the respiratory complaint is bronchiectasis.

52) The method according to claim 44, wherein the respiratory complaint is ARDS (adult respiratory distress syndrome).

53) The method according to claim 44, wherein the respiratory complaint is pulmonary oedema.
